(19) United States
(12) Patent Application Publication
(10) Pub. No.: US 2024/0287594 A1
(43) Pub. Date: Aug. 29, 2024

(54) COMPOSITIONS AND METHODS FOR ENRICHMENT OF NUCLEIC ACIDS USING LIGHT-MEDIATED CROSS-LINKING

(71) Applicant: Dana-Farber Cancer Institute, Inc., Boston, MA (US)

(72) Inventors: Ka Wai Leong, Boston, MA (US); Fangyan Yu, Boston, MA (US); Gerassimos Makrigiorgos, Boston, MA (US)

(21) Appl. No.: 18/571,545

(22) PCT Filed: Jun. 30, 2022
(86) PCT No.: PCT/US22/35727

§ 371 Date: Dec. 18, 2023

Related U.S. Application Data
(60) Provisional application No. 63/327,013, filed on Apr. 4, 2022.
(60) Provisional application No. 63/216,837, filed on Jun. 30, 2021.

(51) Int. Cl. C12Q 1/6858; C12Q 1/6806

(52) U.S. Cl. CPC C12Q 1/6858; C12Q 1/6806

(57) ABSTRACT

Provided herein are methods and compositions for selectively enriching nucleic acid molecules having a target allele sequence within a population of nucleic acid molecules by hybridizing the nucleic acid molecules with an oligonucleotide described herein.

### RELATED APPLICATIONS

This application claims the benefit of U.S. Provisional Application No. 63/216,837, filed on 30 Jun. 2021, and U.S. Provisional Application No. 63/327,013, filed on 4 Apr. 2022; the entire contents of each of said applications are hereby incorporated herein by this reference.

### STATEMENT OF RIGHTS

This invention was made with government support under grant number R01 CA221874 awarded by The National Institutes of Health. The government has certain rights in the invention.

### BACKGROUND OF THE INVENTION

A commonly encountered situation in genetic analysis entails the need to identify a low percent of variant DNA sequences (‘minority alleles’) in the presence of a large excess of non-variant sequences (‘majority alleles’). Examples for such situations include: (a) identification and sequencing of a few mutated alleles in the presence of a large excess of normal (wild type) alleles, a commonly encountered situation in cancer; (b) identification of a few methylated alleles in the presence of a large excess of unmethylated alleles (or: vice versa) in epigenetic analysis; (c) identification and genotyping of a few fetal DNA sequences circulating in the maternal blood where a large excess of maternal DNA sequences are also present; and (d) identification of tumor-circulating DNA in blood or in urine of cancer patients (or abnormal DNA in people suspected of having cancer) in the presence of a large excess of wild type alleles.

While reliable high throughput screening methods for germline or high-prevalence somatic mutations have been described (see Thomas, R. K., Baker, A. C., Debiasi, R. M., Winckler, W., Laframboise, T., Lin, W. M., Wang, M., Feng, W., Zander, T., Macconnaill, L. E. et al. (2007) High-throughput oncogene mutation profiling in human cancer. Nat Genet, 39, 347-351; Chou, L. S., Lyon, E. and Wittwer, C. T. (2005) A comparison of high-resolution melting analysis with denaturing high-performance liquid chromatography for mutation scanning: cystic fibrosis transmembrane conductance regulator gene as a model. Am J Clin Pathol, 124, 330-338; and Thomas, R. K., Nickerson, E., Simons, J. F., Janne, P. A., Tengs, T., Yuza, Y., Garraway, L.A., Laframboise, T., Lee, J.C., Shah, K. et al. (2006) Sensitive mutation detection in heterogeneous cancer specimens by massively parallel picoliter reactor sequencing. Nat Med, 12, 852-855), detection of low-prevalence somatic mutations in tumors with heterogeneity, stromal contamination or in bodily fluids is still problematic. Yet, the clinical significance of identifying these mutations is very important in several situations. For example, in lung adenocarcinoma, low-level EGFR mutations that cannot be identified by regular sequencing can confer either a positive response to tyrosine kinase inhibitors or drug resistance. As additional examples, mutations in plasma useful as biomarkers for early detection or tumor response to treatment cannot be sequenced using conventional methods. Additionally, mutations in tumors with frequent stromal contamination, such as pancreatic or prostate cancer, may be ‘masked’ by presence of wild type alleles, thus requiring laborious micro-dissection in order to correctly detect such mutations or result in missing mutations altogether. For more details regarding exemplary contexts where the methods described herein may be applied, please see Paez, J. G., Janne, P.A., Lee, J.C., Tracy, S., Greulich, H., Gabriel, S., Herman, P., Kaye, F. J., Lindeman, N., Boggon, T. J. et al. (2004) EGFR Mutations in Lung Cancer: Correlation with Clinical Response to Gefitinib Therapy. Science, 304, 1497-1500; Janne, P. A., Borras, A. M., Kuang, Y., Rogers, A. M., Joshi, V. A., Liyanage, H., Lindeman, N., Lee, J. C., Halmos, B., Maher, E. A. et al. (2006) A rapid and sensitive enzymatic method for epidermal growth factor receptor mutation screening. (lin Cancer Res, 12, 751-758; and Engelman, J. A., Mukohara, T., Zejnullahu, K., Lifshits, E., Borras, A. M., Gale, C. M., Naumov, G. N., Yeap, B. Y., Jarrell, E., Sun, J. et al. (2006) Allelic dilution obscures detection of a biologically significant resistance mutation in EGFR-amplified lung cancer. J Clin Invest.).

Beyond cancer, low levels of target DNA in the presence of high levels of non-target DNA occurs in many other fields and applications. For example, detection of fetal alleles within maternal alleles is especially important for prenatal diagnosis during early stages in pregnancy where fetal alleles comprise a low fraction of overall DNA. Another area of interest is detection of low-level mutated alleles in infectious diseases, in order to detect the onset of mutated strains that provide treatment resistance (e.g., antibiotic resistance or vaccine resistance).

In many mutant detection applications, the mutant alleles are detected following a PCR step that amplifies both mutant and wild type alleles. Methods have also been described to preferentially amplify the mutated alleles over wild type alleles (e.g., COLD-PCR). Enrichment that may be obtained via traditional PCR-based methods has drawbacks since polymerase unavoidably introduces mis-incorporations (PCR errors) after several cycles of synthesis, which are subsequently scored as mutations and comprise false positives.

In addition, repeated PCR/COLD-PCR cycles may also introduce mis-priming which results in the amplification of unwanted off-target sequences. Methods that reduce the amount of PCR performed, or can operate without PCR, or in conjunction with PCR if chosen so, have also been developed (e.g., Nuclease-assisted Mutation Enrichment with Probe Overlap, NaME-PrO, that results in selective degradation of target wild type DNA or RNA, thereby providing enrichment of mutated target sequences). NaME-PrO may also be applied to detect low levels of un-methylated DNA alleles in a background of methylated alleles (or vise-versa) (Liu, Y., Song, C., Ladas, I., Fitarelli-Kiehl, M. and Makrigiorgos, G. M. (2017) Methylation-sensitive enrichment of minor DNA alleles using a double-strand DNA-specific nuclease. Nucleic Acids Res, 45, e39). NaME-PrO may be used before, during or after PCR, depending on the application. If NaME-PrO is used for degrading wild-type DNA prior to PCR amplification, most of the PCR-introduced DNA errors are avoided, however, in this method, there is a risk of also enzymatically degrading a fraction of the mutated alleles when these are present at low copy numbers in genomic DNA.

Alternate mutation enrichment methods include enzymatic approaches that may be applied during PCR, or wild type ‘blocker’ approaches that enrich mutated alleles during PCR, such as LNA/PNA-modified oligonucleotides. Once included in the reaction, such amplification-blockers operate during all PCR cycles and there is no ability to increase or reduce their effects to match the template build-up during amplification. Addition of high blocker concentrations can occasionally reduce amplification even for mutated alleles, thus case-specific optimization is required to enable adequate wild type suppression while still allowing enough amplification of rare mutant alleles. Thus, there remains a need for novel and inventive methods of detecting the presence of mutated alleles in biological samples.

### SUMMARY OF THE INVENTION

In some aspects, provided herein are methods comprising: a) placing a photo-reactive molecule in an oligonucleotide in a position suitable for reaction with a non-target nucleic acid sequence present within a population of nucleic acid molecules upon hybridization of the oligonucleotide to the non-target nucleic acid sequence, wherein 1) the photo-reactive molecule has reaction-selectivity towards specific nucleotides, optionally wherein the nucleotides are pyrimidines; and 2) the placement of the photoreactive molecule is opposite position −1, position +1, and/or position zero of a putative alteration following hybridization of the oligonucleotide to a nucleic acid sequence, thereby generating a sequence mismatch at the DNA base-pair right next to the photoactivatable molecule due to the putative alteration (i.e., it is not the photo-reactive molecule that generates the mismatch since the mismatch would be present anyway following hybridization with a mutated sequence, but the photo-reactive molecule is deliberately placed right next to this mismatch);

b) applying light to the population of hybridized oligonucleotide-nucleic acid molecules at a wavelength adapted to induce a covalent cross-link between the photo-activatable molecule and nucleic acid molecules having non-target allele sequence when said nucleic acid molecules having non-target allele sequences are present in the population of nucleic acid molecules; and c) amplifying the population of nucleic acid molecules to form a detectable number of amplified nucleic acid sequences, wherein the presence of the covalent cross-link between the photo-activatable molecule and nucleic acid molecules having non-target allele sequences when said nucleic acid molecules having non-target allele sequences are present in the population of nucleic acid molecules inhibits amplification thereof, optionally, wherein the amplification is digital amplification and/or detecting the amplified nucleic acid molecules.

In other aspects, provided herein are methods of selectively enriching nucleic acid molecules having a target allele sequence within a population of nucleic acid molecules. In some embodiments, the methods comprise hybridizing the nucleic acid molecules with an oligonucleotide that comprises a sequence substantially complementary to at least a portion common between the target allele sequence and a non-target allele sequence, and comprises a photo-activatable molecule.

Numerous embodiments are further provided that may be applied to any aspect encompassed by the present invention and/or combined with any other embodiment described herein. For example, in one embodiment, the photo-activatable molecule may comprise a photo-activatable nucleoside that is complementary to a pyrimidine at nucleotide position −1, on the strand of a nucleic acid molecule having a non-target allele sequence hybridized to the oligonucleotide (e.g., wherein the non-target allele sequence is hybridized to the oligonucleotide). In some embodiments, the photo-activatable molecule comprises a nucleoside conjugated to a 3-cyanovinylcarbazole (CNVK), pyranocarbazole, a psoralen-based molecule, or coumarin-based molecule that is complementary to a pyrimidine, thymidine, cytosine, and/or a methylated cytosine at nucleotide position −1, position +1, and/or position zero on the strand of a nucleic acid molecule having a non-target sequence hybridized to the oligonucleotide (e.g., a non-target allele sequence when the non-target allele sequence is hybridized to the oligonucleotide), thereby generating a sequence mismatch at the DNA base-pair right next to the photoactivatable molecule due to the putative alteration. In some embodiments, the oligonucleotide has X number of sequence mismatches with the non-target allele sequence and has at least X+1 number of sequence mismatches with the target allele sequence, that is to be enriched relative to the non-target allele sequence (e.g., unmutated form of the target allele sequence). In some embodiments, the photo-activatable molecule is placed on the oligonucleotide probe at a distant position relative to the mutated nucleotide of the target allele sequence. In some embodiments, the photo-activatable molecule is placed on the oligonucleotide directly opposite the −1 position of a pyrimidine on the non-target (e.g., wild-type) nucleic acid, such that there is a sequence mismatch at the −1 position relative to the photoactivatable molecule on the oligonucleotide and the target (e.g., mutated) allele sequence, while there is no mismatch with the non-target (e.g., wild-type) allele sequence at that position. Similar positioning of directly opposite positions at −1, zero, and +1 are contemplated for use, especially depending on the preferred photo-activatable molecule used. Without being bound by theory, it has been observed and described herein that a mismatch at the set position (e.g., −1 position), which is supposed to cross-link, diminishes even further the probability of crosslinking with a photo-activator, such as CNVK. This is not just because of reducing hybridization of the oligonucleotide, but also because the crosslinking of the photo-activator with its reacting partner (e.g., a pyrimidine) on the opposite strand was observed to depend strongly on the immediate proximity between the two reacting molecules and a fully-matched sequence.

Additionally, in view of this proximity dependence of cross-linking between the photoactivatable molecule and the nucleic acid base on the opposite strand, performing the cross-linking reaction in solution buffers that enhance the effect of mismatches, such as low monovalent or divalent cation buffers (e.g., sodium Na+0-50 mM concentration; or Mg++0-2 mM concentration) are anticipated to exaggerate even more this effect and to reduce even more the reactivity of the photo-crosslinking molecule with the base on the opposite DNA strand of a target molecule, while retaining substantial reactivity with the corresponding base of a non-target molecule.

Methods described herein may further comprise applying light to the population of hybridized oligonucleotide-nucleic acid molecules at a wavelength adapted to induce a covalent cross-link between the photo-activatable molecule on the oligonucleotide and nucleic acid molecules having non-target allele sequence, while avoiding cross-link with said nucleic acid molecules having target allele sequences (e.g., mutations of interest) that may be present in the population of nucleic acid molecules. Additionally, the method may comprise amplifying the population of nucleic acid molecules to form a detectable number of amplified nucleic acid sequences, wherein the presence of the covalent cross-link between the photo-activatable molecule and nucleic acid molecules having non-target allele sequences prohibits from amplification the cross-linked nucleic acid molecules (e.g., when said nucleic acid molecules having non-target allele sequences are present in the population of nucleic acid molecules inhibits amplification thereof), optionally, wherein the amplification is digital amplification, thereby selectively enriching the nucleic acid molecules having a target allele sequence within the population of nucleic acid molecules. In some embodiments, the nucleic acid amplification comprises polymerase chain reaction (PCR), optionally wherein nucleic acid molecules having target sequences are exponentially amplified and nucleic acid molecules having non-target allele sequences are linearly amplified when said nucleic acid molecules having non-target allele sequences are present in the population of nucleic acid molecules. The PCR amplification may be selected from the group consisting of COLD-PCR, touch-down PCR, arbitrarily-primed PCR (AP-PCR), quantitative reverse transcription PCR (RT-qPCR), digital PCR (dPCR), asymmetric PCR, and solid-support based PCR (e.g., PCR on beads, PCR on slides, etc.).

In some embodiments, the nucleic acid amplification comprises isothermal amplification. The isothermal amplification may be recombinase-polymerase amplification (RPA), LAMP-isothermal amplification, or strand displacement amplification.

In some embodiments, the methods described herein further comprise a step of depleting non-target nucleic acid molecules from the population of nucleic acid molecules before oligonucleotide hybridization comprising performing oligonucleotide hybridization with the population of nucleic acid molecules wherein the oligonucleotide carrying the photo-activatable molecule is immobilized on a solid support. In some embodiments, the method further comprises applying light to the population of hybridized oligonucleotide-nucleic acid molecules to induce a covalent cross-link between the photo-activatable molecule and nucleic acid molecules having non-target allele sequence when said nucleic acid molecules having non-target allele sequences are present in the population of nucleic acid molecules, and removing the solid supports comprising cross-linked nucleic acid molecules having non-target allele sequence from the population of nucleic acid molecules. The methods provided herein may include a step of depleting non-target nucleic acid molecules from the population of nucleic acid molecules. This method may comprise, for example, performing oligonucleotide hybridization with the population of nucleic acid molecules, wherein the oligonucleotide is conjugated to a binding moiety, such as a biotin moiety, applying light to the population of hybridized oligonucleotide-nucleic acid molecules to induce a covalent cross-link between the photo-activatable molecule, and removing the (biotinylated) cross-linked nucleic acid molecules having the non-target allele sequence from the population of nucleic acid molecules.

In some embodiments, the method further comprises repeating the step of depleting non-target nucleic acid molecules from the population of nucleic acid molecules at least 2 times to at least 100 times and/or depleting non-target nucleic acid molecules from the population of nucleic acid molecules by at least 1-, 2-, 3-, 4-, 5-, 6-, 7-, 8-, 0-, 10-, 11-, 12-, 13-, 14-, 15-, 16-, 17-, 18-, 19-, 20-, 25-, 30-, 35-, 40-, 45-, 50-, 55-, 60-, 65-, 70-, 75-, 80-, 85-, 90-, 95-, 100-fold, or more, or any range in between, inclusive, such as 10-fold to 50-fold. The solid support may be a bead (e.g., a streptavidin bead), and/or the oligonucleotide may be biotinylated.

In some embodiments, the light is applied before, concurrently with, or after any step of PCR thermocycling. The light may be applied more than once, optionally the light may be applied more than once before, concurrently with, or after any step of PCR thermocycling.

In some aspects, provided herein are methods of selectively enriching nucleic acid molecules having a target allele sequence within a population of nucleic acid molecules.

As described above, numerous embodiments are further provided that may be applied to any aspect encompassed by the present invention and/or combined with any other embodiment described herein. For example, in one embodiment, methods described herein may further comprise hybridizing the nucleic acid molecules with an oligonucleotide (e.g., an oligonucleotide that is immobilized on a solid support, an oligonucleotide that is conjugated to a biotin moiety, and the like). The oligonucleotide may comprise a sequence substantially complementary to at least a portion common between the target allele sequence and a non-target allele sequence; and a photo-activatable molecule. In some embodiments, the photo-activatable molecule comprises a photo-activatable nucleoside that is complementary to a pyrimidine at nucleotide position −1 on either the strand of a nucleic acid molecule having a non-target allele sequence hybridized to the oligonucleotide or the strand of a nucleic acid molecule having a target allele sequence hybridized to the oligonucleotide. In some embodiments, the photo-activatable molecule comprises a nucleoside conjugated to a-cyanovinylcarbazole (CNVK), pyranocarbazole, a psoralen-based molecule or coumarin-based molecule that is complementary to a thymidine or cytosine or methylated cytosine at nucleotide position −1 on either the strand of a nucleic acid molecule having a non-target allele sequence hybridized to the oligonucleotide or the strand of a nucleic acid molecule having a target allele sequence hybridized to the oligonucleotide.

Similarly, in some embodiments, if the oligonucleotide comprising the photo-activatable molecule hybridizes to the strand of the nucleic acid molecule having the non-target allele sequence, the method further comprises applying light to the population of hybridized oligonucleotide-nucleic acid molecules at a wavelength adapted to induce a covalent cross-link between the photo-activatable molecule and nucleic acid molecules having non-target allele sequence when said nucleic acid molecules having non-target allele sequences are present in the population of nucleic acid molecules. The method may further comprise removing the non-target sequences cross-linked to the oligonucleotide (e.g., an oligonucleotide that is immobilized on a solid support, an oligonucleotide that is conjugated to a biotin moiety, and the like), thereby enriching the nucleic acid molecules having a target allele sequence within the population of nucleic acid molecules.

In other embodiments, if the oligonucleotide comprising the photo-activatable molecule hybridizes to the strand of the nucleic acid molecule having the target allele sequence, the method further comprises applying light to the population of hybridized oligonucleotide-nucleic acid molecules at a wavelength adapted to induce a covalent cross-link between the photo-activatable molecule and nucleic acid molecules having target allele sequence; removing the target sequences cross-linked to the oligonucleotide (e.g., an oligonucleotide that is immobilized on a solid support, an oligonucleotide that is conjugated to a biotin moiety, and the like); and applying light to the population of hybridized oligonucleotide-nucleic acid molecules at a wavelength adapted to reverse the covalent cross-link between the photo-activatable molecule and nucleic acid molecules having target allele sequence, thereby enriching the nucleic acid molecules having a target allele sequence within the population of nucleic acid molecules.

The methods described herein may further comprise a step of amplifying the nucleic acid molecules having the target allele sequence after removing the target sequences cross-linked to the oligonucleotide.

In some embodiments, the oligonucleotide comprising the photo-activatable molecule hybridizes to the strand of the nucleic acid molecule having the non-target allele sequence, and the oligonucleotide has X number of sequence mismatches with the non-target allele sequence and has at least X+1 number of sequence mismatches with the target allele sequence. In other embodiments, the oligonucleotide comprising the photo-activatable molecule hybridizes to the strand of the nucleic acid molecule having the target allele sequence, and the oligonucleotide has X number of sequence mismatches with the target allele sequence and has at least X+1 number of sequence mismatches with the non-target allele sequence. In some embodiments, there is a sequence mismatch at the −1, zero, and/or +1 position of the target (e.g., mutated) allele sequence, but no mismatch at the corresponding-1, zero, and/or +1 position of the non-target (e.g., wild-type) allele sequences, such as at the −1 position for CNVK), for example, generating a sequence mismatch at the DNA base-pair right next to the photoactivatable molecule due to the putative alteration. As described above, it is demonstrated herein that the mismatch opposite the photo-activatable molecule unexpectedly significantly reduces the ability to cross-link such that placement of the photo-activatble molecule at that exact position (e.g., at a-1 position relative to the target allele position desired to be enriched) provides unexpected benefits.

In some embodiments, the method described herein further comprise a step of nucleic acid amplification before oligonucleotide hybridization. The nucleic acid amplification may comprise a polymerase chain reaction (PCR) to form a detectable number of amplified nucleic acid sequences. The step of nucleic acid amplification prior to oligonucleotide hybridization may comprise amplifying the population of nucleic acid molecules with polymerase chain reaction (PCR) using a single amplification primer comprising a 5′ tail and DNA polymerase to form a detectable number of nucleic acid sequences comprising the target allele sequence. In some embodiments, the step of nucleic acid amplification before oligonucleotide hybridization comprises using terminal deoxynucleotidyl-transferase to add poly-adenine tails to the 3′ end of the nucleic acid molecules.

In some embodiments, the oligonucleotide is between about 5 and 200 bp in length. The target allele may be selected from the group consisting of a single nucleotide polymorphism (SNP), a micro-deletion and an insertion. The target allele sequence and the non-target allele sequence may differ by more than one single nucleotide. In some embodiments, the method further comprises incorporating a modified DNA base into the nucleic acid molecules prior to oligonucleotide hybridization, optionally wherein the modified DNA base is selected from the group consisting of methylated deoxy-cytosine-triphosphate (d^{5m}CTP), deoxyuridine triphosphate (dUTP), deoxyinosine triphosphate (dITP), ‘N-methyladenine triphosphate, 8-oxo-guanine triphosphate, +N-methylcytosine triphosphate, 5-substituted pyrimidine trisphosphate and 7-substituted 7-deazapurine triphosphate.

In some embodiments, the method further comprises treating the nucleic acid molecules prior to oligonucleotide hybridization with bisulfite (e.g., sodium bisulfite), methyl-seq, or APOBEC. The method may further comprise hybridizing a second oligonucleotide described herein to the nucleic acid molecules, but the second oligonucleotide hybridizes to the complementary sequence strand of the non-target allele sequence.

The pyrimidine in any one of the methods disclosed herein may be methylated or unmethylated.

In some embodiments, the oligonucleotide comprising the photo-activatable molecule is a split probe, and the split probe comprises a region which is substantially complementary to the non-target allele sequence, operatively linked to a poly-adenine linker and a nucleotide region that is complementary to a portion common between the target allele sequence and a non-target allele sequence.

In some embodiments, the method described herein comprises adding a nucleotide tail to the nucleic acid molecules prior to oligonucleotide hybridization, optionally wherein the nucleotide tail addition is performed through ligation or by extension with a PCR primer.

In some embodiments, nucleic acids from a sample (e.g., a biological sample disclosed herein, such as a sample comprising genomic DNA) may be fragmented. In some embodiments, the nucleic acid sequences may be ligated to adapter sequences, which then may bind primers in amplification of nucleic acid sequences (e.g., target or non-target nucleic acid sequences).

In some embodiments, the oligonucleotide comprising the photo-activatable molecule is a split probe, and wherein the split probe comprises a region which is substantially complementary to the non-target allele sequence, operatively linked to a poly-adenine linker, and a nucleotide region complementary to the nucleotide tail.

The methods provided herein comprise a step of generating the single-stranded nucleic acid molecules by denaturing double-stranded nucleic acid molecules, optionally wherein the double-stranded nucleic acid molecules are genomic DNA.

The methods provided herein further comprise a step of detecting and/or quantifying the enriched nucleic acid molecules having the target allele sequences. Quantifying may comprise Sanger sequencing, high resolution melting (HRM), single strand confirmation polymorphism (SSCP), next generation sequencing, MALDI-TOF, single molecule sequencing, massively parallel sequencing (MPS), or third generation sequencing. A step of detecting and/or quantifying may comprise the use of microarrays (e.g., any microarray disclosed herein).

The population of nucleic acid molecules may be obtained by lysing cells, optionally wherein the cells are lysed with chemical(s) comprising one or more detergents, mechanical disruption, sonication, and/or freezing and thawing. The cells may be in a form selected from the group consisting of cultured cells, biopsies, fresh cells, formalin-fixed paraffin-embedded (FFPE) cells, paraffinized cells, and frozen cells.

As described above, numerous embodiments are further provided that may be applied to any aspect encompassed by the present invention and/or combined with any other embodiment described herein. For example, in one embodiment, the photo-activatable molecule on the oligonucleotide is placed at a position suitable for direct, covalent photo cross-linking with the altered nucleotide on a non-target allele sequence, while avoiding cross-linking with the target sequence in view of (a) removal of a pyrimidine; and/or (b) creation of a mismatch at the position of the alteration to prevent cross-linking due to reduced proximity with the target nucleotide. The photo-activatable nucleoside may be a 3-cyanovinylcarbazole phosphoramidite (CNVK), pyranocarbazole, a psoralen-based molecule or coumarin-based molecule.

The light may be ultraviolet (UV) light or visible light. The methods may comprise enriching for multiple target sequences, either by designing multiple oligonucleotides targeting different nucleic acid sequences or designing an oligonucleotide with a generic portion.

The target nucleic acid sequence may be a portion of an oncogene (e.g., KRAS, TP53, or BRAF) or a fetal gene. The target nucleic acid sequence may comprise KRAS mutations G12S, G12V, G12A or G13D. In some embodiments, the target nucleic acid sequence comprises a minority allele (e.g., an allele that confers disease resistance, such as an EGFR allele). In some embodiments, the target nucleic acid sequence comprises an epimutation (e.g., a mutation that confers a change in methylation status of a base). In some embodiments, the target nucleic acid sequence comprises a tumor associated antigen (e.g., a MAGE, such as MAGE A1).

In some embodiments, the oligonucleotide comprises a modified nucleic acid disclosed herein. The modified nucleic acid may be peptide nucleic acid, locked nucleic acid, ‘super A,G,C,T’, and/or an intercalator that binds to the minor DNA groove, such as a MGB probe.

In some embodiments, the methods further comprises an additional oligonucleotide (e.g., an oligonucleotide comprising a photo-activatable molecule), such as two or more, three or more, four or more or five or more oligonucleotides. One of ordinary skill in the art readily understands that multiple (one or more) oligonucleotides disclosed herein may be used in the methods disclosed herein. Use of multiple probes may be used to improve the discrimination and or specificity of the methods disclosed herein. Therefore, in some embodiments, two or more, three or more, four or more, five or more, six or more, seven or more, eight or more, nine or more, or ten or more oligonucleotides disclosed herein may be used in the enrichment or amplification methods disclosed herein. In some embodiments, two or more, three or more, four or more, five or more, six or more, seven or more, eight or more, nine or more, or ten or more oligonucleotides disclosed herein may be used to target a single nucleic acid target sequence. In some embodiments, two or more, three or more, four or more, five or more, six or more, seven or more, eight or more, nine or more, or ten or more oligonucleotides disclosed herein may be used to target a one or more nucleic acid target sequences. In some embodiments, two or more oligonucleotides are directed against different methylated C positions on a single target. In some embodiments, one or more oligonucleotides comprise multiple photo-activatable moieties, such that they are directed simultaneously against more than one methylated C position on a single target.

In some embodiments, at least one, at least two, at least three, at least four, at least five, at least six, at least seven, at least eight, at least nine, at least ten inosines are added to the oligonucleotides disclosed herein. Inosines may be added to the 3′ end of the oligonucleotide. Inosine is known to pair with all 4 DNA nucleotides, A, T, C and G. In some embodiments, the inosines are consecutive to each other. In other embodiments, the inosines are non-consecutive to each other, optionally containing one, two, three, four, or five or more DNA nucleotides (e.g., native DNA nucleotides) between each inosine.

In some embodiments, the DNA comprising target and/or non-target sequences can be fragmented prior to amplification according to any method known in the art. In some embodiments, the amplification reaction is done after crosslinking, and certain PCR dyes, such as LC green, are added after UV irradiation and crosslinking. Exemplary, non-limiting methods disclosed herein can be found in Ka Wai Leong, Fangyan Yu, G Mike Makrigiorgos, Mutation enrichment in human DNA samples via UV-mediated cross-linking, Nucleic Acids Research, Volume 50, Issue 6, 8 Apr. 2022, Page e32, which is hereby incorporated by reference in its entirety.

### BRIEF DESCRIPTION OF FIGURES

FIG. 1A-FIG. 1C show a schematic diagram of exemplary UV-mediated cross-linking Minor-allele Enrichment (UVME) PCR reactions. FIG. 1A shows a UV lamp attached to a thermal cycler to provide UV irradiation during PCR. The UV lamp may be manually switched on/off anytime during PCR cycling to provide UV irradiation to DNA samples. FIG. 1B shows CNVK-modified probes that are applied in UVME-PCR. Target specific CNVK-modified probes are designed to match the wild type (WT) sense strand while forming a mismatch with mutated sense DNA strands. Common CNVK-modified probes matching both wild type and mutated DNA are designed for the anti-sense DNA strand. When UV is applied, the hybridized CNVK-modified probes cross-link with T or C on the opposite DNA strand at the −1 position. FIG. 1C shows UVME-PCR applied in two stages. First, some (e.g., 10-20) cycles of regular closed lid PCR are performed to build-up target DNA copies from genomic DNA. Optionally, the pre-amplification may be performed via COLD-PCR to amplify preferentially mutated DNA which further enhances the mutation enrichment. For more details regarding COLD-PCR, see Li, J., Wang, L., Mamon, H., Kulke, M. H., Berbeco, R. and Makrigiorgos, G.M. (2008) Replacing PCR with COLD-PCR enriches variant DNA sequences and redefines the sensitivity of genetic testing. Nat Med, 14, 579-584 and Li, J., Wang, L., Janne, P. A. and Makrigiorgos, G.M. (2009) Coamplification at lower denaturation temperature-PCR increases mutation-detection selectivity of TaqMan-based real-time PCR. Clin Chem, 55, 748-756. In the second stage of UVME-PCR, the lid of the PCR machine is opened, and the UV lamp is attached. Following denaturation, CNVK-modified probes and primers bind to their corresponding sequences during annealing. The target specific CNVK-modified probes hybridize to the sense strand wild type DNA preferentially as compared to the sense strand mutated DNA, and application of UV blocks subsequent polymerization. Meanwhile, the common CNVK-modified probes bind equally to both wild type and mutant antisense template and block antisense strand polymerization. Accordingly, UV-mediated cross-linking reduces amplification of both strands wild type DNA but only one strand mutated DNA, in each PCR cycle, resulting to robust mutation enrichment during PCR.

FIG. 2A and FIG. 2B show results of an exemplary application of UVME-PCR to enrich KRAS exon 2 mutations (G12S, G12V, G12A and G13D). UVME-PCR was tested with serial dilutions of genomic DNA from four KRAS-mutated cell lines (A549-G12S, SW480-G12V, H2009-G12A and LOVO-G13D) into wild type DNA. A single pair of KRAS target specific CNVK-modified probes and KRAS common CNVK-modified probes was used for all four mutations. FIG. 2A shows UVME-PCR products screened via TaqMan® genotyping assays reveal significant enrichment and mutation identification down to 0.1% and 0.01% MAF on G12S/G13D and G12V/G12A respectively. FIG. 2B shows UVME-PCR products screened via digital droplet PCR genotyping quantifies mutations down to 0.01% MAF for all four KRAS mutations.

FIG. 3A-FIG. 3D show results of an exemplary application of UVME-PCR to a p53 mutation, C275G. UVME-PCR was tested with serial dilutions of genomic DNA containing a hotspot TP53 mutation (PFSK cell line, C275G) into wild type DNA. Serial dilutions of PFSK into Human male genomic DNA (HMC) DNA down to 0.001% MAF were formed, with the lowest dilutions using an input of 1 μg DNA. FIG. 3A shows results of UVME-PCR followed by Sanger sequencing reveals that mutations in dilutions down to 0.1% were detectable following UV irradiation, while no mutations are evident without UV application. FIG. 3B shows that UVME-PCR followed by high resolution melting (HRM) discriminates mutations down to 0.001% from wild type samples following UV irradiation. FIG. 3C shows UVME-PCR followed by TaqMan® genotyping show that application of UV increases mutation abundance from 0.01%-1% to 3%-80%, respectively. FIG. 3D shows UVME followed by ddPCR detects mutations down to 0.001% and increases mutation abundance from 0.001%-1% to 1%-80%, respectively.

FIG. 4A and FIG. 4B show results of an exemplary UVME-PCR applied to DNA extracted from ten lung tumor tissue samples previously shown to harbor medium to low KRAS G12D, G12V or G13D mutations, as well as four samples with wild type KRAS. Human male genomic DNA was also screened as a control. FIG. 4A shows that ddPCR was first applied to detect the mutation abundance in the absence of UVME enrichment and mutations in 1-40% abundance was demonstrated. ddPCR applied following UVME-PCR resulted to an increase in mutation abundance. No mutations were detected for the four wild type samples and HMC DNA. FIG. 4B shows that UVME-PCR followed by Sanger sequencing depicts mutations for all six samples harboring KRAS mutations; mutations could not be detected on five out of six positive samples in the absence of UV irradiation, using Sanger sequencing.

FIG. 5A and FIG. 5B show a representative UVME-PCR setup performed on an Eppendorf™ Mastercycler™ Nexus GX2 thermal cycler that enables opening of the machine lid without interrupting the PCR program. Open-lid PCR was performed, as described. FIG. 5A shows an illustrative open-lid PCR setup involving UV irradiation by a UV lamp (average wavelength ˜365 nm) that was placed in a standard position on-top of PCR tubes. FIG. 5B shows wells from D-G and 1-4 were shown to provide uniform UV irradiation across the 16 tubes. These positions were used for all UV irradiation experiments.

FIG. 6 shows results of representative UVME-PCR followed by TaqMan® genotyping assays for four KRAS mutations revealing significant shifts for 1% mutation frequency. UVME-PCR was applied on 100% wild type and 1% KRAS mutation-containing DNA, followed by TaqMan® genotyping assays. The growth curves and PCR thresholds are consistent with mutation enrichment in the presence of UV irradiation.

FIG. 7 shows results of representative UVME-PCR followed by TaqMan® genotyping assays (e.g., a comparison of one versus CNVK probes per reaction). UVME-PCR was applied on 100% wild type and 1% mutation-containing DNA in the presence of one CNVK probe directed to the sense strand, vs. two CNVK probes directed to both sense and anti-sense strands. Both approaches lead to mutation enrichment as indicated by the TaqMan®-derived mutation abundance in presence or absence of UV irradiation. However, including both CNVK-modified probes shows higher enrichment than using a single probe directed to the sense strand, for all four KRAS mutations.

FIG. 8 shows that UVME-PCR followed by direct Sanger sequencing yields a detectable mutant signal down to 0.3% for four KRAS mutations assayed. UVME-PCR was applied on genomic DNA containing dilutions (MAF 1%-0.01%) of KRAS mutated genomic DNA followed by direct Sanger sequencing, without secondary amplification. All four KRAS genomic DNA samples showed detectable mutations down to 0.3% mutation input after UV light was applied. No mutation was evident in the absence of UV irradiation or for wild type DNA.

FIG. 9 shows that UVME-PCR followed by direct HRM can detect mutations down to 0.1% for all of four KRAS mutations assayed. UVME-PCR was applied on genomic DNA containing dilutions (MAF 1%-0.01%) of KRAS mutated genomic DNA followed by direct HRM, without secondary amplification. All four KRAS genomic DNA samples showed detectable mutations down to 0.1% mutation input when UV was applied. No mutation is evident in the absence of UV irradiation or for wild type DNA.

FIG. 10 shows plots of mutation abundance following UVME-PCR. Plots show mutation abundance following UVME-PCR compared to the original mutation abundance formed by serial dilution of mutated and wild type genomic DNA. The data indicate that mutation abundance of genomic DNA after UVME-PCR correlates with mutation abundance shown in starting genomic DNA for all four mutations.

FIG. 11A and FIG. 11B show that applying UVME-TT-fast-COLD-PCR boosts mutation enrichment for KRAS, G12S, G12V and G13D, UVME-TT-fast-COLD-PCR was applied to boost further the mutation enrichment obtained by UVME-PCR.

FIG. 11A shows TaqMan® genotyping assay results and FIG. 11B shows ddPCR results. Both methodologies showed higher enrichment for 0.01% G12S, G12V and G13D via UVME-TT-fast-COLD-PCR as compared to UVME-PCR. The significance (*) was calculated by comparing UV to NO UV samples.

FIG. 12 shows an exemplary method of pyrimidine-dependent UVME for detecting a BRAF V600E (T >A) mutation.

FIG. 13 shows a representative application of pyrimidine-specific UV-mediated cross-linking of wild type target using UVME probes during the annealing stage of PCR cycling. BRAF T>A mutation is shown as example.

FIG. 14A and FIG. 14B show selective enrichment of a target mutated allele (red curve) after cross-linking of the wild type allele (blue curve), as described in Example 3 (sense-strand amplification). For example, FIG. 14B shows that an initial 5% mutation abundance is enriched to >80% mutation abundance.

FIG. 15 shows a representative PCR validation of pyrimidine-specific UV-mediated cross-linking of wild type target using UVME probes.

FIG. 16A and FIG. 16B show application of pyrimidine-specific UV-mediated cross-linking of mutated DNA upon hybridization to probes bound to magnetic beads (no PCR required).

FIG. 17 shows an exemplary method of mismatch-dependent UVME for detecting a BRAF V600E (T >A) mutation.

FIG. 18A and FIG. 18B show representative embodiments for preparation and use of split-CNVK probes for mutation enrichment using mismatch-dependent UVME.

FIG. 18A shows application of a mismatch-dependent UVME approach using split-CNVK probes. FIG. 18B shows synthesis of target-specific CNVK probes. These probes are generated from a generic CNVK containing oligo that need only be synthesized once.

FIG. 19 shows application of an exemplary UVME approach to enrich un-methylated or methylated nucleic acid targets. Following bisulfite treatment the methylated C nucleotides remain as C, while un-methylated C becomes uracils (or T after amplification). Probes are designed to match fully methylated targets, whil forming mismatches with un-methylated alleles. Application of UVME during PCR enriches the un-methylated alleles. The opposite may be applied to enrich methylated alleles, as desired.

FIG. 20A and FIG. 20B show results of clinical tissue samples tested using mismatch-dependent UVME-PCR for mutation enrichment followed by direct Sanger sequencing/ddPCR. The utility of UV-based mutation enrichment is demonstrated in these figures. UVME-PCR was applied to DNA from five samples of tumor lung tissue that were shown to harbor medium to low KRAS G12D, G12V or G13D mutations. DNA from two tumor lung tissues and human male DNA were employed as controls. FIG. 20A shows results of UVME-PCR following Sanger sequencing and demonstrates detectable mutations from all five samples but not two wild type samples. FIG. 20B shows results of ddPCR applied to validate mutation abundance. TL8 shows enrichment on both G12V and G13D after UVME-PCR. Moreover, corresponding enrichment is shown on TL3, TL5 and TL6 by UVME-PCR. No enrichment was shown in control DNA (i.e., TL80, TL81) and human male DNA.

FIG. 21 shows a plot of mutation abundance following application of an exemplary UVME-PCR embodiment. Mutation abundance following UVME-PCR is compared against original mutation abundance formed by serial dilution of mutated and wild type genomic DNA. The data indicate that mutation abundance of genomic DNA after UVME-PCR correlates with mutation abundance in starting genomic DNA for all four mutations.

FIG. 22 shows a non-limiting, representative embodiment of a UVME approach to enrich either methylated or un-methylated targets directly from genomic DNA without bisulfite or other chemical treatment. The CNVK-probes are biotinylated and designed to bind a plurality of sequences commonly present within CpG-rich genomic sites. CNVK is placed opposite potentially methylated C within target sites, such as CpG sites. Application of UVME in this representative example results in biotinylated oligonucleotides crosslinked to sequences with methylated Cs, which enables streptavidin-bead based separation of methylated sites on genomic fractions containing the generic CpG sequences interrogated by CNVK probes. Subsequent release from streptavidin beads and PCR enables selective detection of methylated sites using, for example, sequencing or microarray technology. Conversely, unmethylated sites can be directly PCR-amplified and sequenced following UVME, since the methylated targets are ‘blocked’ by the probes and cannot be amplified via direct PCR.

FIG. 23 shows a non-limiting, representative example of enrichment of methylated genomic fraction via direct UV crosslinking of 5mC. This example uses methods disclosed in Example 13.

FIG. 24 shows a non-limiting, representative example of a design for CNVK probes that bind to a palindromic GC-rich target. The design reduces excessive probe-to-probe interaction and increases probe-to-target binding.

FIG. 25A and FIG. 25B show non-limiting, representative enrichment of unmethylated MAGE-A1 sequences in presence of excess methylated alleles following UVME cross-linking of the methylated alleles. FIG. 25A shows results of unmethylated MAGE ultramers (sequences containing TGT) were synthesized by IDT and mixed with methylated MAGE ultramers (sequences containing AGA). The methylated alleles contained methylated CpG nucleotides. UVME probes containing CNVK were used to crosslink selectively the methylated sequences at the position of the CpG nucleotides via 8 cycles of [UV irradiation 10 see then denaturation at 950C 1 see, then repeat the cycle]. Following this, samples were PCR amplified in order to selectively amplify alleles that were not cross-linked by CNVK probes. Subsequent Sanger sequencing demonstrated strong enrichment of the unmethylated sequences (containing TGT) and thus validating the selective crosslinking of the methylated alleles (containing AGA). Exemplary enrichment was enabled by the use of two CNVK probes directed against different methylated C positions on a single target. Two CNVK probes were used to bind simultaneously different methylated C positions on MAGE-A1. UV was then applied to selectively crosslink one or both methC, thus inhibiting amplification. FIG. 25B shows certain results using colon tumors, which have been reported to harbor unmethylated MAGE-A1 DNA, whereas normal colon contains only methylated MAGE-A1. It is demonstrated that UV cross-linking of methylated CGs at positions 161 or 129 of the MAGE-A1 gene enables subsequent enrichment of the unmethylated alleles following PCR amplification. To validate this point, following direct UV-mediated crosslinking of genomic DNA from tumor CT19, obtained from a colon cancer patient, bisulfite conversion was applied followed by PCR of the bisulfite treated DNA. It can be seen that in the absence of UV crosslinking (top electropherogram) there is an equal amount of methylated and unmethylated Cs at the positions of CG dinucleotides (highlighted by blue squares). When UV-crosslinking is applied and the process repeated, only the unmethylated alleles can be amplified, as the methylated alleles are inhibited and prevented from PCR amplification. This is another exemplary demonstration of the ability of the method to amplify and enrich unmethylated alleles of clinical relevance.

For any figure showing a bar histogram, curve, or other data associated with a legend, the bars, curve, or other data presented from left to right for each indication correspond directly and in order to the boxes from top to bottom of the legend.

### DETAILED DESCRIPTION OF THE INVENTION

This invention provides, in part, novel enrichment methods which enable detection, amplification and/or enrichment of target alleles (e.g., mutated alleles), referred to as UV-mediated Cross-linking Minor-allele Enrichment (UVME). The enrichment of target (e.g., mutated) alleles can take place when target alleles are within a high excess of non-target (e.g., wild-type) alleles. The methods provided herein may be performed at any stage during amplification, optionally performed via polymerase chain reaction (PCR), as well as prior to or after PCR. In some embodiments, the methods described herein enable wild type, DNA blocking to start/stop at any stage during PCR, as well as pre-PCR or post-PCR, thus providing the most flexible and practical approach to mutation enrichment to-date. Methods described herein may also be used to provide enrichment of methylated or un-methylated alleles, thus enabling application in the field of DNA methylation. Methods provided herein may also be used to enrich of methylated or un-methylated alleles.

The present invention has a number of unexpected advantages. The methods, as described herein, increase the current detection limits of nucleic acid mutation and/or methylation detection. The enrichment methods provided herein also enable high multiplexity of targets, thus enabling high-throughput methods to be used, for example, for somatic mutation detection. Additionally, the methods described herein allow mutation detection in a time-, cost-, and labor-effective manner. In some embodiments, the enrichment methods described herein may be used to detect mutations in nucleic acid target sequences. In some embodiments, the enrichment methods described herein may be used to detect nucleic acid base methylation status in nucleic acid target sequences. In some embodiments, the nucleic acid target sequences are genomic DNA. The nucleic acid sequences described herein (e.g., those containing target sequences to be detected, amplified and/or enriched) may be isolated or derived from any biological sample that comprises such nucleic acids, including, but not limited to biological samples such as tumor samples (e.g., a heterogeneous tumor sample), urine, sweat, saliva and sputum.

### I. Oligonucleotides Comprising Photo-Activatable Molecules

In some aspects, provided herein are methods of selectively enriching nucleic acid molecules having a target allele sequence within a population of nucleic acid molecules by hybridizing the nucleic acid molecules with an oligonucleotide described herein.

Provided herein are oligonucleotides comprising photo-activatable molecules for use in the methods provided herein. In some embodiments, the oligonucleotides are nucleic acids that comprise a sequence substantially complementary to at least a portion common between a target allele sequence (e.g., a sequence comprising a mutation of interest or a methylated/non-methylated base of interest) and a non-target allele sequence. In some embodiments, the photo-activatable molecule is capable of cross-linking with a specific nucleotide base when exposed to light (e.g., UV light or visible light). A photo-activatable molecule, as described herein, includes any molecule that forms covalent bonds with other molecules (e.g., such as DNA base pairs) when exposed to light. In some embodiments, the photo-activatable molecule comprises a photo-activatable nucleoside, such as 3-cyanovinylcarbazole phosphoramidite (CNVK), pyranocarbazole, or a nucleoside conjugated to coumarin. For example, CNVK is a nucleoside that may be photo-activated by UV irradiation at 365 nm. Therefore, in some embodiments, the light is UV light, and the light is applied at 365 nm. In some embodiments, cross-linking between the oligonucleotide and either the target or non-target sequence is reversed. For example, exposure of the cross-link between a CNVK containing oligonucleotide and a nucleic acid sequence may be reversed by application of UV light at 312 nm. Therefore, the methods described herein encompass reversal of the crosslink, such as application of UV light at 312 nm. Additionally, such photo-activatable molecules described herein can exhibit preferences for cross-linking with specific nucleic acid bases. For example, CNVK forms covalent cross-links with pyrimidine bases cytosine and thymine, as well as modified DNA bases such as methylated cytosine. Indeed, CNVK and pyranocarbazole form cross-links preferentially with the following bases in decreasing order: methylated cytosine, thymine, and unmethylated cytosine. When a CNVK-containing nucleoside is incorporated into an oligonucleotide described herein, it allows UV-enabled cross-linking with a preferred base at −1 position of the opposite nucleic acid strand. In a similar fashion, pyranocarbazole nucleoside exhibits the same base preference as CNVK. Pyranocarbazole nucleoside forms covalent cross-links with DNA bases when exposed to visible light. Additional details regarding pyranocarbazole nucleoside may be found at Fujimoto et al. Org. Lett. 2018, 20, 2802-2805, hereby incorporated by reference in its entirety. Coumarin exhibits a preference for thymidine. The methods described herein, in part, utilize these base preferences to enrich target nucleic acid molecules.

In some embodiments, the oligonucleotide is designed to preferentially hybridize and cross-link with a non-target allele. In some embodiments, the oligonucleotide is designed to preferentially hybridize and cross-link with a target allele. The oligonucleotides described herein may be between about 5 bp and about 200 bp in length, such as 5 bp to 20 bp, 5 bp to 25 bp, 10 bp to 50 bp, 25 bp to 50 bp, 30 bp to 50 bp, 40 bp to 60 bp, 50 bp to 100 bp, 75 bp to 100 bp, 100 bp to 125 bp, 125 bp to 150 bp, 150 bp to 175 bp, or 175 bp to 200 bp in length. In some embodiments, the oligonucleotide is a short probe. For example, the oligonucleotides described herein may be less than 20 bp in length, such as less than 19, less than 18, less than 17, less than 16, less than 15, less than 14, less than 13, less than 12, less than 11, less than 10, less than 9, less than 8, less than 7, less than 6, or less than 5 bp in length.

In some embodiments, the oligonucleotide has X (e.g., at least one, two, three, four, five, six, seven, eight, nine, ten, eleven, twelve, thirteen, fourteen, fifteen, twenty, twenty-five, thirty, forty or fifty) sequence mismatches with the non-target allele sequence and has at least X+1 sequence mismatches with the target allele sequence. In some embodiments, the oligonucleotide has X (e.g., at least one, two, three, four, five, six, seven, eight, nine, ten, eleven, twelve, thirteen, fourteen, fifteen, twenty, twenty-five, thirty, forty or fifty) sequence mismatches with the target allele sequence and has at least X+1 sequence mismatches with the non-target allele sequence. Sequence mismatches are designed to optimize differential hybridization of the oligonucleotide to either the target or non-target sequence, depending on the method of enrichment employed. A person of ordinary skill in the art will appreciate that fewer mismatches between a nucleic acid sequence and the oligonucleotide will generally result in increased hybridization, whereas an increase in the number of mismatches with result in less hybridization. It is described herein that, in some embodiments, when the photoactivatable molecule is placed exactly opposite the position of the altered nucleotide (e.g., −1, +1, and/or zero) in the target sequence, such that a mismatch is formed at the corresponding position(s) by the altered target sequence, there is a major drop in the generation of cross-linking upon photo-activation. This guides the preferred placement of the photoactivatable molecule on the oligonucleotide in order to prevent cross-linking with the target allele sequence, while allowing crosslinking with the non-target allele sequence. In some embodiments, the target allele sequence and the non-target allele sequence differ by more than one, more than two, more than three, more than four, more than five, more than six, or more than ten nucleotides. Oligonucleotides may comprise a modified base.

In some embodiments, the oligonucleotide comprises a photo-activatable molecule. In some embodiments, the photo-activatable molecule is a photo-activatable nucleoside that is complementary to a pyrimidine at nucleotide position −1 on the strand of a nucleic acid molecule having a non-target allele sequence hybridized to the oligonucleotide. In some embodiments, the photo-activatable molecule is a nucleoside conjugated to a coumarin molecule that is complementary to a thymidine at nucleotide position −1 on the strand of a nucleic acid molecule having a non-target allele sequence hybridized to the oligonucleotide.

In some embodiments, the oligonucleotide comprising a photo-activatable molecule is a split probe. Split probes include any oligonucleotide comprising two nucleic acid regions joined by a linker. In some embodiments, the split probe comprises a linker (e.g., a poly-adenine linker), which is operatively linked to a nucleotide region that is complementary to a portion common between the target allele sequence and a non-target allele sequence. In other embodiments, the split probe comprises a region which is substantially complementary to the non-target allele sequence, operatively linked to a linker (e.g., a poly-adenine linker), and a nucleotide region complementary to a generic nucleotide tail. In such cases, a nucleotide tail is added to nucleic acid molecules prior to oligonucleotide hybridization. Addition of the nucleotide tail may be performed through ligation or by extension with a PCR primer.

Oligonucleotides may be synthesized and prepared by any suitable method (such as chemical synthesis), which is known in the art. A number of computer programs (e.g., Primer-Express) are readily available to design oligonucleotides according to the design and structural parameters described herein. Additional modifications to the oligonucleotides consistent with the embodiments of this invention may be found in Part III: General Methods and Compositions.

An oligonucleotide can comprise a modified backbone and/or modified internucleoside linkages. Modified backbones can include those that retain a phosphorus atom in the backbone and those that do not have a phosphorus atom in the backbone. Suitable modified oligonucleotide backbones containing a phosphorus atom therein can include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and amino alkylphosphoramidates, phosphorodiamidates, thionophosphoramidates, thionoalkylphosphonates, thionoalkylphosphotriesters, sclenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. Suitable oligonucleotides having inverted polarity can comprise a single 3′ to 3′ linkage at the 3′-most internucleotide linkage (i.e., a single inverted nucleoside residue in which the nucleobase is missing or has a hydroxyl group in place thereof). Various salts (e.g., potassium chloride or sodium chloride), mixed salts, and free acid forms may also be included. An oligonucleotide can comprise one or more phosphorothioate and/or heteroatom internucleoside linkages, in particular-CH2—NH—O—CH₂—,—CH₂—N(CH₃)—O—CH₂—(i.e., a methylene (methylimino) or MMI backbone),—CH₂—O—N(CH₃)—CH₂—,—CH₂—N(CH₃)—N(CH₃)—CH₂— and —O—N(CH₃)—CH₂—CH₂—(wherein the native phosphodiester internucleotide linkage is represented as-O-P(=O)(OH)—O—CH₂—).

An oligonucleotide can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage.

An oligonucleotide can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

An oligonucleotide can comprise a nucleic acid mimetic. The term “mimetic” may be intended to include polynucleotides wherein only the furanose ring or both the furanose ring and the internucleotide linkage are replaced with non-furanose groups, replacement of only the furanose ring may also be referred as being a sugar surrogate. The heterocyclic base moiety or a modified heterocyclic base moiety may be maintained for hybridization with an appropriate target nucleic acid. One such nucleic acid may be a peptide nucleic acid (PNA). In a PNA, the sugar-backbone of a polynucleotide may be replaced with an amide containing backbone, in particular an aminoethylglycine backbone. The nucleotides may be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties may be bound directly or indirectly to aza nitrogen atoms of the amide portion of the backbone.

An oligonucleotide can comprise linked morpholino units (i.e., morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides may be nonionic mimics of oligonucleotides. A variety of compounds within the morpholino class may be joined using different linking groups. A further class of polynucleotide mimetic may be referred to as cyclohexenyl nucleic acids (CeNA). The furanose ring normally present in a nucleic acid molecule may be replaced with a cyclohexenyl ring. CeNA DMT protected phosphoramidite monomers may be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage may be a methylene (—CH₂—), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

An oligonucleotide can comprise one or more substituted sugar moieties. Suitable polynucleotides can comprise a sugar substituent group selected from: OH; F; O—, S—, or N—alkyl; O—, S—, or N-alkenyl; O—, S—or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O((CH₂) ₙO)ₘCH₃, O(CH₂)ₙOCH₃, O(CH₂)ₙNH₂, O(CH₂)ₙnCH₃, O(CH₂)ₙONH₂, and O(CH₂) NON((CH₂)ₙCH₃)₂, where n and m are from 1 to about 10. A sugar substituent group may be selected from: C₁ to C₁₀ lower alkyl, substituted lower alkyl, alkenyl, alkynyl, alkaryl, aralkyl, O-alkaryl or O-aralkyl, SH, SCH₃, OCN, CI, Br, CN, CF₃, OCF₃, SOCH₃, SO₂CH₃,ONO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification can include 2′-methoxyethoxy(2′-O—CH₂CH₂OCH₃, also known as 2′-O—(2-methoxyethyl) or 2′-MOE (i.e., an alkoxyalkoxy group). A further suitable modification can include 2′-dimethylaminooxyethoxy (i.e., a O(CH₂)₂ON(CH₃)₂ group, also known as 2′-DMAOE), and 2′-dimethylaminoethoxyethoxy (also known as 2′-O-dimethyl-amino-ethoxy-ethyl or 2′-DMAEOE) (i.e., 2′-O—CH₂O—CH₂—N(CH₃)₂).

Other suitable sugar substituent groups can include methoxy (—O—CH₃), aminopropoxy (—OCH₂CH₂CH₂NH₂), allyl (—CH₂—CH(=CH₂)),—O-allyl (—O—CH₂—CH(=CH₂)) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked nucleotides and the 5′ position of 5′ terminal nucleotide. Oligomeric compounds may also have sugar mimetics such as cyclobutyl moieties in place of the pentofuranosyl sugar.

A oligonucleotide may also include nucleobase (often referred to simply as “base”) modifications or substitutions. As used herein, “unmodified” or “natural” nucleobases can include the purine bases, (e.g. adenine (A) and guanine (G)), and the pyrimidine bases, (e.g., thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2—thiouracil, 2-thiothymine and 2-thiocytosine, 5-halouracil and cytosine, 5-propynyl (—C(═C-CH₃)) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b)(1,4)benzoxazin-2(3H)-one), carbazole cytidine (2H-pyrimido(4,5-b)indol-2-one), pyridoindole cytidine (Hpyrido(3′,2′:4,5)pyrrolo(2,3-d)pyrimidin-2-one).

Heterocyclic base moieties can include those in which the purine or pyrimidine base is replaced with other heterocycles, for example 7-deaza-adenine, 7-deazaguanosine, 2—aminopyridine and 2-pyridone. Nucleobases may be useful for increasing the binding affinity of a polynucleotide compound. These can include 5-substituted pyrimidines, 6-azapyrimidines and N-2, N-6 and O—6 substituted purines, including 2-aminopropyladenine, 5-propynyluracil and 5-propynylcytosine. 5-methylcytosine substitutions can increase nucleic acid duplex stability by 0.6-1.2° C. and may be suitable base substitutions (e.g., when combined with 2′-O-methoxyethyl sugar modifications).

A modification of an oligonucleotide can comprise chemically linking to the oligonucleotide one or more moieties or conjugates that can enhance the activity, cellular distribution or cellular uptake of the oligonucleotide. These moieties or conjugates can include conjugate groups covalently bound to functional groups such as primary or secondary hydroxyl groups. Conjugate groups can include, but are not limited to, intercalators, reporter molecules, polyamines, polyamides, polyethylene glycols, polyethers, groups that enhance the pharmacodynamic properties of oligomers, and groups that can enhance the pharmacokinetic properties of oligomers. Conjugate groups can include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that can enhance the pharmacokinetic properties include groups that improve uptake, distribution, metabolism or excretion of a nucleic acid. Conjugate moieties can include but are not limited to lipid moieties such as a cholesterol moiety, cholic acid, a thioether (e.g., hexyl-S-tritylthiol), a thiocholesterol, an aliphatic chain (e.g., dodecandiol or undecyl residues), a phospholipid (e.g., di-hexadecyl-rac-glycerol or triethylammonium 1,2-di-O-hexadecyl-rac-glycero-3-H-phosphonate), a polyamine or a polyethylene glycol chain, or adamantane acetic acid, a palmityl moiety, or an octadecylamine or hexylamino-carbonyl-oxycholesterol moiety.

### II. Enrichment Methods

### A. Nucleic Acid Target Sequences

The methods provided herein are methods to enrich target nucleic acid sequences (e.g., a sequence comprising a mutation or interest or a methylated/non-methylated nucleic acid base). In some embodiments, the nucleic acid target sequence is in an oncogene. In some embodiments, the nucleic acid target sequence is in a BRAF gene. In some embodiments, the nucleic acid target sequence is in a KRAS gene. In some embodiments, the nucleic acid target sequence is in a TP53 gene. In some embodiments, the nucleic acid target sequence is in a EGFR gene. The nucleic acid target sequence may comprise a single nucleotide polymorphism (SNP), a micro-deletion, or an insertion. For example, the nucleic acid target sequence may comprise any mutation that has deleted a thymine or cytosine or any mutation that has inserted a thymine or cytosine. The nucleic acid target sequence may comprise a SNP mutation, such as one that has converted a thymine to a cytosine, adenine, or guanine. The nucleic acid target sequence may comprise a mutation that has converted a cytosine to adenine or guanine. In some embodiments, the nucleic acid target sequence is in a fetal gene. In some embodiments, the nucleic acid target sequence comprises a low copy number allele. In some embodiments, the nucleic acid target sequence comprises a minority allele.

As used herein, “thymine” may be written at “T”, cytosine may be written as “C”, adenine may be written as “A” and guanine may be written as “G”.

Many nucleic acid molecule types exist within cells and certain types that can comprise a mutation of interest. The term “nucleic acid molecules” or “nucleic acids” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. The terms “ribonucleic acid” and “RNA” as used herein mean a polymer composed of ribonucleotides. The terms “deoxyribonucleic acid” and “DNA” as used herein mean a polymer composed of deoxyribonucleotides.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. In one embodiment, nucleic acid molecules are genomic DNA or are derived from genomic DNA, such as fragments or chromosomes. Such genomic DNA can comprise exome DNA, i.e., a subset of whole genomic DNA enriched for transcribed sequences which contains the set of exons in a genome. In one embodiment, nucleic acid molecules are RNA.

The nucleic acid sequences (e.g., target sequences comprising a mutation of interest or a non-target wild type sequence) for use in the methods of the present invention may be derived using well-known methods in the art. In some embodiments, the nucleic acid sequences are generated in vitro. In other embodiments, the nucleic acid sequences are obtained from biological sources in the form of samples comprising such sequences. In either case, the term “sample” is used herein in a broad sense and is intended to include a variety of sources and compositions that contain such nucleic acid sequences. Thus, the sample may be a biological sample, but the term also includes other, for example, artificial samples which comprise nucleic acids. Exemplary samples include, but are not limited to, whole blood; blood products such as plasma or serum; red blood cells; white blood cells; buffy coat; swabs, including but not limited to buccal swabs, throat swabs, vaginal swabs, urethral swabs, cervical swabs, throat swabs, rectal swabs, lesion swabs, abscess swabs, nasopharyngeal swabs, and the like; urine; sputum; saliva; semen; lymphatic fluid; amniotic fluid; cerebrospinal fluid; peritoneal effusions; pleural effusions; fluid from cysts; synovial fluid; vitreous humor; aqueous humor; bursa fluid; eye washes; eye aspirates; pulmonary lavage; lung aspirates; tissues, including but not limited to, liver, spleen, kidney, lung, intestine, brain, heart, muscle, pancreas, cell cultures, plant tissues or samples, as well as lysates, extracts, or materials and fractions obtained from the samples described above or any cells and microorganisms and viruses that may be present on or in a sample and the like. Materials obtained from clinical or forensic settings that contain nucleic acids are also within the intended meaning of the term “sample.” In one embodiment, nucleic acid sources from biological sources, such as subjects, having a particular condition, such as cancer, and/or treated under a particular condition, such as with a therapeutic or modulator of a biological process, may be used. Non-limiting examples of such samples include frozen tissue samples, fresh tissue samples, paraffin-embedded samples, and samples that have been preserved, e.g. formalin-fixed and paraffin-embedded (FFPE samples) or other samples that were treated with cross-linking fixatives such as, for example, glutaraldehyde.

As described above, the term “sample” also includes processed samples such as preserved, fixed and/or stabilized samples. As described herein, suitable samples useful for extracting nucleic acid molecules to be fragmented according to the methods of the present invention described herein can contain biological material retrieved from a host organism of 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, or longer before the methods of the present invention are applied.

The methods according to the present invention are particularly useful for enriching target nucleic acid sequences from tissue samples, whole tissue, whole organ, bodily fluids, tumor dissections, cell culture, cell lysate, cell extract, and the like. In some embodiments, the biological sample comprises or is obtained from a “population of cells,” which term indicates at least two cells.

Moreover, in some embodiments, biological samples and/or nucleic acid sequences therein may be pooled in any combination of interest, such as pooling of cells of the same type and/or after the same treatment, pooling of cells of different types and/or different treatment, and the like.

For those embodiments where biological samples are used to obtain the nucleic acid sequences, such as whole cells or tissue samples, it is generally useful to extract the sequences from other biological material in order to generate a population of nucleic acids molecules for analysis. Accordingly, following sample collection, nucleic acid molecules may be liberated from the collected cells, biological fluids, etc., into a crude extract, followed by additional treatments to prepare the sample for subsequent operations, e.g., removal of nucleic acids unprotected by a ribosome, purification, filtration, desalting, and the like.

Liberation of nucleic acid sequences from the biological sample may be performed using well-known chemical, physical, or electrolytic lysis methods. For example, chemical methods generally employ lysing agents to disrupt the cells and extract the nucleic acid molecules from cells. Generally, where chemical extraction and/or denaturation methods are used, the appropriate reagents may be incorporated through external introduction of agents to the sample.

Alternatively, physical methods may be used to extract the nucleic acids. For example, U.S. Pat. No. 5,304,487 discusses the use of physical protrusions within microchannels or sharp edged particles within a chamber or channel to pierce cell membranes and extract their contents. Combinations of such structures with piezoelectric elements for agitation can provide suitable shear forces for lysis. Such elements are described in greater detail with respect to nucleic acid fragmentation, below. More traditional methods of cell extraction may also be used, e.g., using mechanical disruption, employing a channel with restricted cross-sectional dimension which causes cell lysis when the sample is passed through the channel with sufficient flow pressure, and the like.

In some embodiments, cell extraction and denaturing of contaminating proteins may be carried out by applying an alternating electrical current to the sample. More specifically, the sample of cells is flowed through a microtubular array while an alternating electric current is applied across the fluid flow. A variety of other methods may be utilized within the device of the present invention to effect cell lysis/extraction, including, e.g., subjecting cells to ultrasonic agitation, freeze-thawing, trituration, or forcing cells through microgeometry apertures, thereby subjecting the cells to high shear stress resulting in rupture.

Following extraction, nucleic acid molecules, but need not, be separated from other elements of the crude extract, e.g., denatured proteins, cell membrane particles, salts, and the like. Removal of particulate matter is generally accomplished by centrifugation, filtration, flocculation, or the like. A variety of filter types may be readily incorporated into the device. Further, where chemical denaturing methods are used, it may be desirable to desalt the sample prior to proceeding to subsequent steps. Desalting of the sample, and isolation of the nucleic acid may generally be carried out in a single step, e.g., by binding the nucleic acids to a solid phase and washing away the contaminating salts or performing gel filtration chromatography on the sample, passing salts through dialysis membranes, and the like. Suitable solid supports for nucleic acid binding include, e.g., diatomaceous earth, silica (i.e., glass wool), or the like. Suitable gel exclusion media, also well known in the art, may also be readily incorporated into the devices of the present invention, and is commercially available from, e.g., Pharmacia and Sigma Chemical.

The isolation and/or gel filtration/desalting may be carried out in an additional chamber, or alternatively, the particular chromatographic media may be incorporated in a channel or fluid passage leading to a subsequent reaction chamber. Alternatively, the interior surfaces of one or more fluid passages or chambers may themselves be derivatized to provide functional groups appropriate for the desired purification, e.g., charged groups, affinity binding groups and the like.

Alternatively, desalting methods may generally take advantage of the high electrophoretic mobility and negative charge of DNA compared to other elements. Electrophoretic methods may also be utilized in the purification of nucleic acids from other cell contaminants and debris. In one example, a separation channel or chamber of the device is fluidly connected to two separate “field” channels or chambers having electrodes, e.g., platinum electrodes, disposed therein. The two field channels are separated from the separation channel using an appropriate barrier or “capture membrane” which allows for passage of current without allowing passage of nucleic acids or other large molecules. The barrier generally serves two basic functions: first, the barrier acts to retain the nucleic acids which migrate toward the positive electrode within the separation chamber; and second, the barriers prevent the adverse effects associated with electrolysis at the electrode from entering into the reaction chamber (e.g., acting as a salt junction). Such barriers may include, e.g., dialysis membranes, dense gels, PEI filters, or other suitable materials. Upon application of an appropriate electric field, the nucleic acids present in the sample will migrate toward the positive electrode and become trapped on the capture membrane. Sample impurities remaining free of the membrane are then washed from the chamber by applying an appropriate fluid flow. Upon reversal of the voltage, the nucleic acids are released from the membrane in a substantially purer form. The field channels may be disposed on the same or opposite sides or ends of a separation chamber or channel, and may be used in conjunction with mixing elements described herein, to ensure maximal efficiency of operation. Further, coarse filters may also be overlaid on the barriers to avoid any fouling of the barriers by particulate matter, proteins or nucleic acids, thereby permitting repeated use.

In a similar aspect, the high electrophoretic mobility of nucleic acids with their negative charges, may be utilized to separate nucleic acids from contaminants by utilizing a short column of a gel or other appropriate matrix or gel which will slow or retard the flow of other contaminants while allowing the faster nucleic acids to pass.

In some embodiments, it may be desirable to extract certain species of nucleic acids, such as DNA or RNA, species based on size (e.g., genomic, plasmid, transcribed, small, micro, chromosomal, etc.), species based on strandedness (e.g., single stranded or double stranded), species based on composition (e.g., cDNA or cRNA), and the like. Conventional techniques for isolating desired nucleic acids may be used and are well known in the art for example as disclosed in Sambrook and Russell, Molecular Cloning: A Laboratory Manual and as described in the Examples. In some embodiments, the method further comprises a step of generating the single-stranded nucleic acid molecules by denaturing double-stranded nucleic acid molecules, optionally wherein the double-stranded nucleic acid molecules are genomic DNA.

Non-limiting, exemplary techniques include methods of using a cartridge supported with a nucleic acid-adsorbable membrane of silica, cellulose compound, or the like, precipitation with ethanol or precipitation with isopropanol, extraction with phenol-chloroform, and the like. Furthermore, there may be mentioned methods with solid-phase extraction cartridge, chromatography, and the like using ion-exchange resins, silica supports bonded with a hydrophobic substituent such as an octadecyl group, resins having a size-exclusion effect.

The methods of the present invention are robust, such that sample(s) may be subjected to more than one type and/or a repetition of a perturbation to a biological sample to obtain nucleic acid molecules for analysis as needed. For example, chemical (e.g., detergent) lysis of cells may be sufficient to release suitable nucleic acid for analysis without the addition of additional manipulations, such as extensive fluid washing, mechanical disruption, sonication, freezing and thawing, trituration, and the like.

### a) Enrichment Methods Using Amplification

In some aspects, provided herein are methods of selectively enriching nucleic acid molecules having a target allele sequence within a population of nucleic acid molecules by hybridizing the nucleic acid molecules with an oligonucleotide described herein.

In some embodiments, the methods utilize the oligonucleotides described herein to block amplification of the non-target sequence, while allowing exponential amplification of the target sequence. In these embodiments, the oligonucleotide is designed to preferentially hybridize and cross-link with the non-target sequence, such that subsequent amplification of the non-target sequence is blocked by the oligonucleotide. In such methods, light is applied to the population of hybridized oligonucleotide-nucleic acid molecules at a wavelength adapted to induce a covalent cross-link between the photo-activatable molecule within the oligonucleotide and nucleic acid molecules having the non-target allele sequence. Subsequent amplification methods form a detectable number of amplified nucleic acid sequences, wherein the presence of the covalent cross-link between the photo-activatable molecule and nucleic acid molecules having non-target allele sequences selectively enrich the nucleic acid molecules having a target allele sequence within the population of nucleic acid molecules.

The nucleic acid amplification may be any amplification method known in the art. For example, the amplification may be digital amplification. In some embodiments, the nucleic acid amplification comprises polymerase chain reaction (PCR), optionally wherein nucleic acid molecules having target sequences are exponentially amplified and nucleic acid molecules having non-target allele sequences are linearly amplified when said nucleic acid molecules having non-target allele sequences are present in the population of nucleic acid molecules. The PCR amplification may be any PCR amplification described herein or known in the art.

### Non-Limiting Embodiments of these Methods May be Found in the Working Examples

### b) Enrichment Methods That May Be Performed Independent of Amplification

Provided herein are methods of selectively enriching nucleic acid molecules having a target allele sequence within a population of nucleic acid molecules comprising hybridizing the nucleic acid molecules with an oligonucleotide described herein. The oligonucleotide may be immobilized on a solid support or conjugated to a biotin moiety.

In some embodiments, the oligonucleotide comprising the photo-activatable molecule is designed to hybridize to the strand of the nucleic acid molecule having the non-target allele sequence. In these methods, the steps described herein further comprise applying light to the population of hybridized oligonucleotide-nucleic acid molecules at a wavelength adapted to induce a covalent cross-link between the photo-activatable molecule, and removing the non-target sequences cross-linked to the oligonucleotide (e.g., an oligonucleotide immobilized on a solid support or conjugated to a biotin moiety), thereby enriching the nucleic acid molecules having a target allele sequence within the population of nucleic acid molecules.

In other embodiments, the oligonucleotide comprising the photo-activatable molecule is designed to hybridize to the strand of the nucleic acid molecule having the target allele sequence. In these such embodiments, the methods further comprise applying light to the population of hybridized oligonucleotide-nucleic acid molecules at a wavelength adapted to induce a covalent cross-link between the photo-activatable molecule and nucleic acid molecules having target allele sequence, removing the target sequences cross-linked to the oligonucleotide (e.g., an oligonucleotide immobilized on a solid support or conjugated to a biotin moiety), and applying light to the population of hybridized oligonucleotide-nucleic acid molecules at a wavelength adapted to reverse the covalent cross-link between the photo-activatable molecule and nucleic acid molecules having target allele sequence, thereby enriching the nucleic acid molecules having a target allele sequence within the population of nucleic acid molecules.

The oligonucleotide may be attached to any solid support suitable for the methods provided herein. In one embodiment, the oligonucleotide is attached to the solid support through a metallic nanolayer (e.g., cadmium, zinc, mercury, gold, silver, copper, or platinum nanolayer). In certain embodiments, the solid support is a bead or plurality of beads (e.g., a colloidal particle, a metallic nanoparticle or nanoshell, or a latex bead), a flow path in a lateral flow assay device (e.g., a porous membrane), a flow path in an analytical or centrifugal rotor, a blot (e.g., Western blot, a slot blot, or dot blot), or a tube or a well (e.g., in a plate suitable for an ELISA assay or microarray). In certain embodiments, the solid support comprises metal, glass, a cellulose-based material (e.g., nitrocellulose), or a polymer (e.g., polystyrene, polyethylene, polypropylene, polyester, nylon, or polysulfone). In some embodiments, biotinylated oligonucleotides are bound to magnetic streptavidin beads.

It would be understood by a person or ordinary skill in the art that any method of removing or otherwise separating crosslinked target sequences from non-crosslinked sequences may be used to enrich either target or non-target sequences. Therefore, encompassed herein are methods wherein an oligonucleotide is conjugated to a biotin moiety or equivalent, and removed via a solid support, such as streptavidin beads. Conversely, an oligonucleotide may be linked to a solid support and removed from solution after crosslinking to a nucleic acid sequence.

Finally, the methods described in this section may be performed without an amplification step, or may be combined with any amplification step disclosed herein (e.g., an amplification step prior to or after crosslinking).

Non-limiting embodiment of these methods may be found in the working examples.

### III. General Methods and Compositions

The following disclosure describes methods relevant to both enrichment methods dependent on amplification following hybridization and those not dependent on amplification following hybridization. Both methods described in Part II may further comprise a step of nucleic acid amplification either before or after oligonucleotide hybridization. In some embodiments, the nucleic acid amplification comprises polymerase chain reaction (PCR) to form a detectable number of amplified nucleic acid sequences. In some embodiments, PCR amplification is selected from the group consisting of COLD-PCR, touch-down PCR, arbitrarily-primed PCR (AP-PCR), quantitative reverse transcription PCR (RT-qPCR), digital PCR (dPCR), asymmetric PCR, and solid-support based PCR (e.g., PCR on beads, PCR on slides, etc.).

The methods provided herein may also comprise the use of a second oligonucleotide. For example the methods provided herein may comprise hybridizing a second oligonucleotide to the nucleic acid molecules, but the second oligonucleotide hybridizes to the complementary sequence strand of the non-target allele sequence. In some embodiments, the methods provide for the use of multiple oligonucleotides comprising photo-activatable molecules. The method provided herein may be used for enrichment of a single target nucleic acid sequence, or for multiple targets simultaneously. Such methods may include designing multiple oligonucleotide probes for multiple target sequences, each comprising a different mutation.

In some embodiments, provided herein are methods of amplifying only the DNA strand that comprises a portion to be hybridized to the oligonucleotide described herein. In one embodiment, a single primer extension is applied on the opposite DNA strand to create copies of the DNA strand that comprises a portion to be hybridized to the oligonucleotide. This primer extension creates nucleic acid sequence copies containing an oligonucleotide ‘tail’ on the 5′end of the DNA strand. Following purification to remove the tail-containing primer, the enrichment methods described herein may be applied to selectively cross-link the non-target sequence. Subsequently, PCR using the ‘tail’ as one primer and a reverse primer amplifies only the target strand that was not hybridized and cross-linked by the oligonucleotide. In some embodiments, the methods provided herein comprise a step of nucleic acid amplification prior to oligonucleotide hybridization that comprises amplifying the population of nucleic acid molecules with polymerase chain reaction (PCR) using a single amplification primer comprising a 5′ tail and DNA polymerase to form a detectable number of nucleic acid sequences comprising the target allele sequence. Alternatively, following purification to remove the tail-containing primer, the enrichment methods described herein may be applied to selectively cross-link either the non-target sequence or the target sequence to an oligonucleotide immobilized on a solid support or conjugated to a biotin moiety.

In some embodiments, a DNA strand comprising the target sequence is amplified while the opposite strand is not. Any technique known in the art to selectively amplify the DNA strand comprising the target sequence may be used. For example, the methods provided herein may include a step of nucleic acid amplification before oligonucleotide hybridization that comprises using terminal deoxynucleotidyl-transferase to add poly-adenine tails to the 3′ end of the nucleic acid molecules. Following addition of the poly A tails on the 3′end of the nucleic acid molecules, one may apply PCR using a poly-dT reverse primer that binds to poly-A in conjunction with a forward primer that corresponds to the sense DNA strand that is to be selectively cross-linked. Alternatively, one may start with application of the an enrichment method provided herein and apply asymmetric PCR that includes un-equal amounts of the forward vs. reverse primer to amplify preferentially either the sense or anti-sense DNA strands, as needed.

In some embodiments, the hybridization, amplification, and detection steps are performed in the same vessel. In other embodiments, the hybridization is performed in a separate vessel from the amplification and detection steps. In other embodiments, the hybridization and amplification is performed in a separate vessel from the detection steps. Regardless of whether nucleic acid amplification, enrichment and/or detection, are performed independently in different vessels or continuously in the same vessel, many different types of nucleic acid amplification techniques may be used according to the oligonucleotide parameters discussed further below.

For example, in some embodiments, nucleic acid amplification may be accomplished by a variety of methods. The polymerase chain reaction (PCR) and variations thereof is the method most commonly used to amplify specific target DNA sequences although, in some embodiments, other techniques like nucleic acid sequence based amplification (NASBA), ligase chain reaction (LCR), strand displacement amplification (SDA) reaction, transcription mediated amplification (TMA) reaction, and rolling circle amplification (RCA) may be used.

PCR generally refers to a method for amplification of a desired nucleotide sequence in vitro involving introducing a molar excess of two or more extendable oligonucleotide primers to a reaction mixture comprising a sample having the desired target sequence(s), where the primers are complementary to opposite strands of the double stranded target sequence. The reaction mixture is subjected to a program of thermal cycling in the presence of a DNA polymerase, resulting in the amplification of the desired target sequence flanked by the DNA primers.

PCR amplification generally has three phases: exponential phase, linear phase and plateau phase. The exponential phase is the first phase of PCR amplification. During this exponential phase, reaction components are in excess. Assuming 100% reaction efficiency, there is an exact doubling of product each cycle, and the reaction is specific and precise. The linear phase is the second phase of PCR amplification, during which the reaction components are continuously being consumed but become limiting, amplification therefore slows and the reactions become highly variable. The final phase of PCR amplification is the plateau phase. At the plateau phase, the reaction components are insufficient for amplification and very few or no products are being generated.

The technique of PCR is described in numerous publications, including, PCR: A Practical Approach, M. J. McPherson, et al., IRL Press (1991), PCR Protocols: A Guide to Methods and Applications, by Innis, et al., Academic Press (1990), and PCR Technology: Principals and Applications for DNA Amplification, H. A. Erlich, Stockton Press (1989). PCR is also described in many U.S. patents, including U.S. Pat. Nos. 4,683,195; 4,683,202; 4,800,159; 4,965,188; 4,889,818; 5,075,216; 5,079,352; 5,104,792; 5,023,171; 5,091,310; and 5,066,584. The term “PCR fragment” or “reverse transcription-PCR fragment” or “amplicon” refers to a polynucleotide molecule (or collectively the plurality of molecules) produced following the amplification of a particular target nucleic acid. A PCR fragment is typically, but not exclusively, a DNA PCR fragment. A PCR fragment may be single-stranded or double-stranded, or in a mixture thereof in any concentration ratio. A PCR fragment may be about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500 nucleotides in length, or more, or any range in between, inclusive, such as about 20 to about 500 nucleotides in length, about 50 to about 150 nucleotides in length, etc.

A “buffer” is a compound added to an amplification reaction which modifies the stability, activity, and/or longevity of one or more components of the amplification reaction by regulating the pH of the amplification reaction. The buffering agents of the invention are compatible with PCR amplification. Certain buffering agents are well known in the art and include, but are not limited to, Tris, Tricine, MOPS (3-(N-morpholino) propanesulfonic acid), and HEPES (4-(2-hydroxyethyl)-1-piperazineethanesulfonic acid). In addition, PCR buffers may generally contain up to about 70 mM KCl and about 1.5 mM or higher MgCl₂, to about 50-200 mM each of nucleotides dATP, dCTP, dGTP and dTTP. The buffers of the invention may contain additives to optimize efficient reverse transcription-PCR or PCR reaction.

An additive is a compound added to a composition which modifies the stability, activity, and/or longevity of one or more components of the composition. In certain embodiments, the composition is an amplification reaction composition. In certain embodiments, an additive inactivates contaminant enzymes, stabilizes protein folding, and/or decreases aggregation. Exemplary additives that may be included in an amplification reaction include, but are not limited to, betaine, formamide, KCl, CaCl₂, MgOAc, MgCl₂, NaCl, NH₄OAc, Nal, Na(CO₃)₂, LiCl, MnOAc, NMP, trehalose, demethylsulfoxide (“DMSO”), glycerol, ethylene glycol, dithiothreitol (“DTT”), pyrophosphatase (including, but not limited to Thermoplasma acidophilum inorganic pyrophosphatase (“TAP”)), bovine serum albumin (“BSA”), propylene glycol, glycinamide, CHES, Percoll™, aurintricarboxylic acid, TWEEN® 20, TWEEN® 21, TWEEN® 40, TWEEN® 60, TWEEN® 85, Brij 30, NP-40, Triton X-100, CHAPS, CHAPSO, Mackernium, LDAO (N-dodecyl-N,N-dimethylamine-N-oxide), Zwittergent 3-10, Xwittergent 3-14, Xwittergent SB 3-16, Empigen, NDSB-20, T4G32, E. Coli SSB, RecA, nicking endonucleases, 7-deazaG, dUTP, UNG, anionic detergents, cationic detergents, non-ionic detergents, zwittergent, sterol, osmolytes, cations, and any other chemical, protein, or cofactor that may alter the efficiency of amplification. In certain embodiments, two or more additives are included in an amplification reaction.

The term “template” or “template nucleic acid” refers to a plurality of nucleic acid molecules used as the starting material or template for amplification in a PCR reaction or reverse transcription-PCR reaction. Template nucleic acid sequences may include both naturally occurring and synthetic molecules. Exemplary template nucleic acid sequences include, but are not limited to, RNA protected from degradation by association with a ribosome.

A “target sequence”, “target DNA” or “target RNA” or “target nucleic acid”, or “target nucleic acid sequence” refers to a region of a template nucleic acid that is to be analyzed.

The terms “annealing” and “hybridization” are used interchangeably and mean the base-pairing interaction of one nucleic acid with another nucleic acid that results in formation of a duplex, triplex, or other higher-ordered structure. In certain embodiments, the primary interaction is base-specific, e.g., A/T and G/C, by Watson/Crick and Hoogsteen-type hydrogen bonding. In certain embodiments, base-stacking and hydrophobic interactions may also contribute to duplex stability.

As used herein, “stringent conditions” for hybridization refer to conditions under which a nucleic acid having complementarity to a sequence predominantly hybridizes with that sequence, and substantially does not hybridize to off-target sequences, or, in some embodiments, sufficiently hybridizes to a sequence of interest suitable for detection according to the methods of the present invention despite hybridizing in some fashion to off-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. These are preferably capable of hybridising to the reference sequence under highly stringent conditions. Generally, in order to maximize the hybridization rate, relatively low-stringency hybridization conditions are selected: about 20 to 25° C. lower than the thermal melting point (Tm). Generally, in order to require at least about 85% nucleotide complementarity of hybridized sequences, highly stringent washing conditions are selected to be about 5 to 15° C. lower than the Tm. In order to require at least about 70% nucleotide complementarity of hybridized sequences, moderately-stringent washing conditions are selected to be about 15 to 30° C. lower than the Tm. Highly permissive (very low stringency) washing conditions may be as low as 50° C. below the Tm, allowing a high level of mis-matching between hybridized sequences. Those skilled in the art will recognize that other physical and chemical parameters in the hybridization and wash stages may also be altered to affect the outcome of a detectable hybridization signal from a specific level of homology between nucleic acid sequences and probe sequences. In general, preferred highly stringent conditions comprise incubation in 50% formamide, 5×SSC, and 1% SDS at 42° C., or incubation in 5×SSC and 1% SDS at 65° C., with wash in 0.2×SSC and 0.1% SDS at 65° C. For applications involving oligonucleotide binding stringencies in nucleic acid amplification reactions and where washing is not involved, such as in a PCR assay, oligonucleotide binding stringencies are generally dependent upon temperature and high stringency binding occurs when the hybridization temperature is close to the melting temperature, such as within the ranges of melting and anneal temperatures described below.

A “DNA-dependent DNA polymerase activity” refers to the activity of a DNA polymerase enzyme that uses deoxyribonucleic acid (DNA) as a template for the synthesis of a complementary and anti-parallel DNA strand. In certain embodiments, the nucleic acid polymerase is a thermostable polymerase that may have more than one of the above-specified catalytic activities. As used herein, the term “thermostable”, as applied to an enzyme, refers to an enzyme that retains its biological activity at elevated temperatures (e.g., at 55° C. or higher), or retains its biological activity following repeated cycles of heating and cooling.

As used herein, an “amplifying polymerase activity” refers to an enzymatic activity that catalyzes the polymerization of deoxyribonucleotides or ribonucleotides. Generally, the enzyme will initiate synthesis at the 3′-end of the primer annealed to a target nucleic acid template sequence, and will proceed toward the 5′ end of the template strand.

Non-limiting examples of thermostable DNA polymerases may include, but are not limited to, polymerases isolated from the thermophilic bacteria Thermus aquaticus (Taq polymerase), Thermus thermophilus (Tth polymerase), Thermococcus litoralis (Tli or VENT™ polymerase), Pyrococcus furiosus (Pfu or DEEPVENT™ polymerase), Pyrococcus woosii (Pwo polymerase) and other Pyrococcus species, Bacillus stearothermophilus (Bst polymerase), Sulfolobus acidocaldarius (Sac polymerase), Thermoplasma acidophilum (Tac polymerase), Thermus rubber (Tru polymerase), Thermus brockianus (DYNAZYME™ polymerase), Thermotoga maritime (Tma) and other species of the Thermotoga genus (Tsp polymerase), and Methanobacterium thermoautotrophicum (Mth polymerase). The PCR reaction may contain more than one thermostable polymerase enzyme with complementary properties leading to more efficient amplification of target sequences. For example, a nucleotide polymerase with high processivity (the ability to copy large nucleotide segments) may be complemented with another nucleotide polymerase with proofreading capabilities (the ability to correct mistakes during elongation of target nucleic acid sequence), thus creating a PCR reaction that can copy a long target sequence with high fidelity. The thermostable polymerase may be used in its wild type form. Alternatively, the polymerase may be modified to contain a fragment of the enzyme or to contain a mutation that provides beneficial properties to facilitate the PCR reaction. In one embodiment, the thermostable polymerase may be Taq DNA polymerase. Many variants of Taq polymerase with enhanced properties are known and include, but are not limited to AmpliTaq™, AmpliTaq™, Stoffel fragment, SuperTaq™, SuperTaq™ plus, LA Taq™, LApro Taq™, and EX Taq™.

The term “oligonucleotide” refers to a nucleic acid molecule having at least 2 nucleotides covalently linked together. Oligonucleotides are typically from about 5, 6, 7, 8, 9, 10, 12, 15, 25, 30, 40, 50, 60, 70, 80, 90, 100, 150, 200, 250, 300, 350, 400, 450, or more nucleotides in length, up to about 500 nucleotides in length.

Nucleic acids and polynucleotides are polymers of any length, including longer lengths, e.g., greater than 500, 1000, 2000, 3000, 5000, 7000, 10,000, etc. The term “nucleotide” typically refers to a single unit of a polynucleotide, i.e., a monomer. Nucleotides may be ribonucleotides, deoxyribonucleotides, or modified versions thereof. The term “oligonucleotide” encompasses nucleic acid primers and probes.

As used herein, the term “amplification primer” or “PCR primer” or “primer” refers to an enzymatically extendable oligonucleotide that comprises a defined sequence that is designed to hybridize in an anti-parallel manner with a complementary, primer-specific portion of a target nucleic acid sequence. Thus, the primer, which is generally in molar excess relative to its target polynucleotide sequence, primes template-dependent enzymatic DNA synthesis and amplification of the target sequence. A primer nucleic acid does not need to have 100% complementarity with its template subsequence for primer elongation to occur; primers with less than 100% complementarity may be sufficient for hybridization and polymerase elongation to occur provided the penultimate base at the 3′ end of the primer is able to base pair with the template nucleic acid. A PCR primer is preferably, but not necessarily, synthetic, and will generally be approximately about 10 to about 100 nucleotides in length.

The term “probe” comprises a polynucleotide that comprises a specific portion designed to hybridize in a sequence-specific manner with a complementary region of a specific nucleic acid sequence, such as a target nucleic acid sequence. The precise sequence and length of an oligonucleotide probe of the invention depends in part on the nature of the target polynucleotide to which it binds. The binding location and length may be varied to achieve appropriate annealing and melting properties for a particular embodiment. Guidance for making such design choices may be found in many of the well-known references describing real-time nucleic acid amplification techniques, such as TaqMan™ and CataCleave™ assays described in U.S. Pat. Nos. 5,763,181; 6,787,304; and 7,112,422.

The terms “3′ region” and “5′ region” with respect to any nucleic acid described herein, such as an nucleic acid sequence of the present invention, refers to the relative orientation of a particular sequence with respect to the 5′ (upstream)-to-3′ (downstream) organization of nucleic acid polymers. Thus, in one embodiment, a 3′ region indicates a sequence region that is downstream of another sequence on the same nucleic acid molecule. In another embodiment, a 5′ region indicates a sequence region that is upstream of another sequence on the same nucleic acid molecule. In some embodiments, a 3′ region or a 5′ region may be or comprise a sequence at the very 3′ end or 5′ end, respectively, of a nucleic acid molecule.

A primer's thermal melting point (Tm) is the temperature at which about 50% of the primer and its complement are in duplex (e.g., hybridized). The Tm of a double stranded region of an primer may be calculated from the primer sequence using methods that are well-known in the art. For example, the Tm of an primer may be calculated using the following formula: Tm=4° C. x (number of G's and C's in the primer)+2° C. x (number of A's and T's in the primer). This formula is valid for primers having a double stranded region of <14 bases and assumes that the reaction is carried out in the presence of 50 mM monovalent cations. For longer primers having a double stranded region >14 bases, the following formula may be used: Tm=64.9° C. +41° C.×(number of G's and C's in the primer-16.4)/N, where N is the length of the primer. Another commonly used formula takes into account the salt concentration of the reaction (Rychlik and Rhoads (1989) Nucl. Acids Res. 17:8543; PCR Core Systems Technical Bulletin #TB254, Promega Corporation; Sambrook, J., Fritsch, E. F. and Maniatis, T. (1989) Molecular Cloning: A Laboratory Manual, Cold Spring Harbor Laboratory Press, Cold Spring Harbor, N. Y. and Mueller, P. R. et al. (1993) In: Current Protocols in Molecular Biology 15.5, Greene Publishing Associates, Inc. and John Wiley and Sons, New York): Tm=81.5° C.+16.6° C. x (log₁₀[Na]+[K])+0.41° C.×(% GC)-675/N, where N is the number of nucleotides in the primer.

The most sophisticated Tm calculations take into account the exact sequence and base stacking parameters, not just the base composition (as described in Borer et al. (1974) J. Mol. Biol. 86:843; SantaLucia (1998) Proc. Nat. Acad. Sci. USA 95:1460; Allawi and SantaLucia (1997) Biochem. 36:10581; and von Ahsen et al. (1999) (lin. Chem. 45:2094), as Tm=ΔH kcal® C. * Mol AS+R In ([primer]/2)−273.15° C., where ΔH is the enthalpy of base stacking interactions adjusted for helix initiation factors, AS is the entropy of base stacking adjusted for helix initiation factors and for the contributions of salts to the entropy of the system, and R is the universal gas constant (1.987 Cal/° C. mole). This equation, as implemented above, is valid if i) the primer is not self-complementary, ii) the primer concentration is much greater than the target concentration; iii) the primer is an “oligonucleotide” rather than a long polymer; and iv) the salt effects on polymers is significantly different from those on oligos. For self-complementary primer, the denominator of the equation becomes ΔS+R In([primer]/4). If the concentrations are almost equal, the denominator of the equation becomes ΔS+R In([primer]-[target]/2).

Numerous electronic and commercially available tools are available on the World Wide Web for calculating the melting temperatures of primer (see, for example, the OligoAnalyazer 3.1 calculator available on the Integrated DNA Technologies web site).

A primer's annealing temperature is generally about 5° C. below the Tm of the oligonucleotide, although it may be determined to be about 4.9, 4.8. 4.7, 4.6, 4.5, 4.4, 4.3, 4.2, 4.1, 3.9, 3.8, 3.7, 3.6, 3.5, 3.4, 3.3, 3.2, 3.1, 3.0° C., or less, or any range in between, inclusive, such as about 5° C. to about 3° C. below the primer's Tm. Many of the methods for calculating primer Tm may also be useful in calculating Tm of the oligonucleotides comprising a photo-activatable molecule described herein.

In addition to the particular considerations for primer and oligonucleotide design useful for the methods of the present invention described above, well-known considerations in the art are also useful in designing the nucleic acid primers. For example, the sequences for binding target regions may be chosen such that, where possible, the combined effect of nucleic acid amplification is to detect abundance of sequences arising only from the gene of interest. All complementary binding regions in nucleic acid amplification should be of sufficient length for adequate binding of the DNA polymerase enzymes, respectively. Avoiding extreme GC content, hairpin loop structures, dimer formation sequences, and the like may also be used to inform the oligonucleotide design.

A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). The term “perfectly complementary” means that all the contiguous residues of a nucleic acid sequence will hydrogen bond with the same number of contiguous residues in a second nucleic acid sequence. The term “substantially complementary” as used herein refers to a degree of complementarity that is at least about 80%, 81%, 82%, 83%, 84%, 85%, 86%, 87%, 88%, 89%, 90%, 91%, 92%, 93%, 94%, 95%, 96%, 97%, 98%, 99%, or greater, such as 100% identity, over a region of at least about 8, 9, 10, 11, 12, 13, 14, 15, 16, 17, 18, 19, 20, 21, 22, 23, 24, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 20, 125, 130, 135, 140, 145, 150, or more, nucleotides, such as the entire length of a compared nucleic acid sequence, or any range in between, inclusive, such as 13-20 nucleotides, or refers to two nucleic acids that hybridize under stringent conditions. In addition to sequence complementarity, generally, oligonucleotides for use according to the methods of the present invention and having substantial complementarity with a specific sequence specifically and predominantly hybridize with that particular sequence and substantially (e.g., essentially) do not hybridize to other non-complementary sequences under the methods' conditions. However, due to short hybridization lengths involved in the amplification and detection of target nucleic acids, in certain embodiments, it may not be possible to avoid off-target hybridization. Such embodiments are within the scope of the present invention if the off-target sequences recognized do not significantly distract the investigator from analyzing the target sequences.

The term “substantially identical” as used herein refers to a degree of homology that is at least about 80%, 81%, 82%, 83%, 84%, 85%, 86%, 87%, 88%, 89%, 90%, 91%, 92%, 93%, 94%, 95%, 96%, 97%, 98%, 99%, or greater, such as 100% identity, over a region of at least about 8, 9, 10, 11, 12, 13, 14, 15, 16, 17, 18, 19, 20, 21, 22, 23, 24, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 20, 125, 130, 135, 140, 145, 150, or more, nucleotides, such as the entire length of a compared nucleic acid sequence, or any range in between, inclusive, such as 13-20 nucleotides, between two nucleic acid sequences. In embodiments, where an RNA sequence is compared to a DNA sequence, then uridine nucleotides are considered the same as thymidine nucleotides for purposes of sequence comparison and homology determinations.

In some embodiments, oligonucleotides can comprise one or more modifications, such as chemical modifications at the 3′ end, at the 5′ end, internally, within a base, within the backbone, and the like, either alone or in combination, in order to provide the oligonucleotide with a new or enhanced characteristic, such as improved stability or resistance to degradation (see U.S. Pat. Publ. 2016/0108470).

For example, in one embodiment, an oligonucleotide is chemically modified at its 3′-end, such as with a dideoxyribonucleotide, to block the oligonucleotide from participating in primer extension. For example, the 3′-terminus of an oligonucleotide may be capped at the 3′ terminus with a dideoxythymine triphosphate using a Klenow fragment mutant (F762Y) of DNA polymerase I (Escherichia coli) or T7 DNA polymerase (Tabor and Richardson (1995) Proc. Natl. Acad. Sci. USA 92:6339-6343).

In another embodiment, the oligonucleotide may have one or more blocking agents. A blocking agent refers to a nucleotide (or derivatives thereof), modified oligonucleotides and/or one or more other modifications which are incorporated into the nucleic acid inhibitors of the invention to prevent or inhibit degradation or digestion of such nucleic acid molecules by DNase activity.

“Reverse transcription-PCR” or “RT-PCR” is a PCR reaction that uses RNA template and reverse transcriptase(s), or enzyme(s) having reverse transcriptase activity, to first generate a single stranded DNA molecule prior to the multiple cycles of DNA-dependent DNA polymerase primer elongation. Multiplex PCR refers to PCR reactions that produce more than one amplified product in a single reaction, typically by the inclusion of more than two primers in a single reaction.

Exemplary reverse transcriptases include, but are not limited to, the Moloney murine leukemia virus (M-MLV) RT as described in U.S. Pat. No. 4,943,531, a mutant form of M-MLV-RT lacking RNase H activity as described in U.S. Pat. No. 5,405,776, bovine leukemia virus (BLV) RT, Rous sarcoma virus (RSV) RT, Avian Myeloblastosis Virus (AMV) RT and reverse transcriptases disclosed in U.S. Pat. No. 7,883,871.

In some embodiments, reverse transcriptase and PCR are performed in a procedure known as reverse transcription-PCR, which may be carried out as either an end-point or real-time assay. It involves two separate molecular syntheses: (i) the synthesis of cDNA from an RNA template; and (ii) the replication of the newly synthesized cDNA through PCR amplification. In order to address the technical problems often associated with reverse transcription-PCR, a number of protocols have been developed taking into account the three basic steps of the procedure: (a) the denaturation of RNA and the hybridization of reverse primer; (b) the synthesis of cDNA; and (c) PCR amplification. In the so called “uncoupled” reverse transcription-PCR procedure (e.g., two-step reverse transcription-PCR), reverse transcription is performed as an independent step using the optimal buffer condition for reverse transcriptase activity. Following cDNA synthesis, the reaction is adjusted for MgCl₂, and deoxyribonucleoside triphosphate (dNTP) concentrations amongst other conditions to conditions optimal for DNA polymerase activity of the DNA polymerase, such as Taq polymerase, and PCR is carried out according to standard conditions (see U.S. Pat. Nos. 4,683,195 and 4,683,202). By contrast, “coupled” RT PCR methods use a common buffer optimized for reverse transcriptase and DNA polymerase activities. In one embodiment, the annealing of reverse primer is a separate step preceding the addition of enzymes, which are then added to the single reaction vessel. In another version, the reverse transcriptase activity is a component of the thermostable Tth DNA polymerase. Annealing and cDNA synthesis are performed in the presence of Mn²⁺ then PCR is carried out in the absence of Mn²⁺after the removal of Mn²⁺ by a chelating agent. Finally, the “continuous” method (e.g., one step reverse transcription-PCR) integrates the three reverse transcription-PCR steps into a single continuous reaction that avoids the opening of the reaction tube for component or enzyme addition. Continuous reverse transcription-PCR has been described as a single enzyme system using the reverse transcriptase activity of thermostable Taq DNA polymerase and Tth polymerase and as a two enzyme system using AMV RT and Taq DNA polymerase, wherein the initial 65° C. RNA denaturation step may be omitted.

One-step reverse transcription-PCR provides several advantages over uncoupled reverse transcription-PCR. One step reverse transcription-PCR requires less handling of the reaction mixture reagents and nucleic acid products than uncoupled reverse transcription-PCR (e.g., opening of the reaction tube for component or enzyme addition in between the two reaction steps), and is therefore less labor intensive, reducing the required number of person hours. One step reverse transcription-PCR also requires less sample, and reduces the risk of contamination. The sensitivity and specificity of one-step reverse transcription-PCR has proven well suited for studying expression levels of one to several genes in a given sample. Typically, this procedure has been limited to use of gene-specific primers to initiate cDNA synthesis. In such methods, the reverse transcriptase may be heat inactivated before or during heat activation of DNA polymerase.

An oligonucleotide can comprise a detectable label. The terms “label,” “detectable moiety,” “detectable agent,” and like terms refer to a composition detectable by spectroscopic, photochemical, biochemical, immunochemical, chemical, or other physical means. For example, useful labels include fluorescent dyes, luminescent agents, radioisotopes (e.g., 32P, 3H, and the like), electron-dense reagents, enzymes, biotin, digoxigenin, or haptens and proteins or other entities which may be made detectable, e.g., by affinity. The term “tag” may be used synonymously with the term “label,” but generally refers to an affinity-based moiety, e.g., a “His tag” for purification, or a “streptavidin tag” that interacts with biotin.

Any method known in the art for conjugating a nucleic acid or other biomolecule to a label may be employed, e.g., using methods described in Hermanson, Bioconjugate Techniques 1996, Academic Press, Inc., San Diego. For example, labeling may be performed in a non-enzymatic manner. For example, the Universal Labeling System™ (ULS™) technology may be used (ULS™ array CGH Labeling Kit; manufactured by Kreatech Biotechnology BV Company) and the like may be also used. Briefly, ULS™ labeling is based on the stable binding properties of platinum (II) to nucleic acids (van Gijlswijk et al. (2001) Expert Rev. Mol. Diagn. 1:81-91). The ULS molecule consists of a monofunctional platinum complex coupled to a detectable molecule of choice.

As a method for fluorescent labeling, a direct labeling method or an indirect labeling method may be used. The direct labeling method means a method where a nucleic acid is transformed into a single-strand one, a short-chain nucleic acid is hybridized thereto, and a nucleotide compound to which a fluorescent substance (e.g., Cy-dye) has been bound is mixed with the nucleotide, thereby the nucleic acid is labeled in one step. The indirect labeling method means a method where a nucleic acid is transformed into a single-strand one, a short-chain nucleic acid is hybridized thereto, a nucleotide compound having a substituent capable of being bound to a fluorescent substance (e.g., Cy-dye), for example, a nucleotide compound having an aminoallyl group and the natural nucleotide are mixed together, a nucleic acid having the substituent is first synthesized, and then a fluorescent substance (e.g., Cy-dye) is bound through the aminoallyl group, thereby the nucleic acid being labeled. As methods for introducing a labeling compound such as a fluorescent substance into the nucleic acid, a random primer method (primer extension method), a nick translation method, a PCR method, a terminal labeling method, and the like may be used.

Other labeling methods are also well-known. For example, the random primer method is a method where a random primer nucleic acid having several by (base pair) to over ten by is hybridized and amplification and labeling are simultaneously performed using a polymerase, thereby a labeled nucleic acid being synthesized. The nick translation method is a method where, for example, a double-strand nucleic acid to which nick has been introduced with DNase I is subjected to the action of a DNA polymerase to decompose DNA and simultaneously synthesize a labeled nucleic acid by the polymerase activity. The PCR method is a method where two kinds of primers are prepared and a PCR reaction is carried out using the primers, thereby amplification and labeling being simultaneously performed to obtain a labeled nucleic acid. The terminal labeling method is a method where, in a method of labeling a 5′-end, a labeling compound such as a fluorescent substance is incorporated into a 5′-end of a nucleic acid dephosphorylated with an alkaline phosphatase by a phosphorylation reaction with a T4 polynucleotide kinase. A method of labeling 3′-end is a method where a labeling compound such as a fluorescent substance is added to a 3′-end of a nucleic acid with a terminal transferase. As the labeled sample nucleic acid or the like, it is also possible to use an unpurified solution containing the same. In the case of using such an unpurified solution, an enzyme and the like still remain in the solution and hence, after preparation, it is preferable to deactivate the activity of the enzyme remaining in the solution. It is based on the viewpoint of preventing the influence on reproducibility of data. As methods for deactivating the enzyme, any methods may be possible as long as they can deactivate the enzyme but it is preferable to perform any one or both of a method of adding a chelating agent or a heating treatment at 60° C. or higher. The heating temperature is preferably 60° C. or higher. The heating time is sufficiently 1 minute or more and most preferably, it is preferred to perform the heating treatment at 65° C. or higher for 5 minutes or more. Moreover, in the case of labeling method using a Klenow fragment, it is also possible to deactivate the activity of the enzyme using a vortex mixer or the like.

A “labeled” molecule (e.g., nucleic acid, protein, or antibody) is one that is bound, either covalently, through a linker or a chemical bond, or noncovalently, through ionic, van der Waals, electrostatic, or hydrogen bonds to a label such that the presence of the molecule may be detected by detecting the presence of the label bound to the molecule. The signal from the label may be indicative of the amount of the labeled molecule.

One type of label is a fluorochrome. As used herein, “fluorochrome” refers to a fluorescent compound that emits light upon excitation by light of a shorter wavelength than the light that is emitted. The term “fluorescent donor” or “fluorescence donor” refers to a fluorochrome that emits light that is measured in the assays described in the present invention. More specifically, a fluorescent donor provides energy that is absorbed by a fluorescence acceptor. The term “fluorescent acceptor” or “fluorescence acceptor” refers to either a second fluorochrome or a quenching molecule that absorbs energy emitted from the fluorescence donor. The second fluorochrome absorbs the energy that is emitted from the fluorescence donor and emits light of longer wavelength than the light emitted by the fluorescence donor. The quenching molecule absorbs energy emitted by the fluorescence donor.

Any luminescent molecule, preferably a fluorochrome and/or fluorescent quencher may be used in the practice of this invention, including, for example, Alexa Fluor™ 350, Alexa Fluor™ 430, Alexa Fluor™ 488, Alexa Fluor™ 532, Alexa Fluor™ 546, Alexa Fluor™ 568, Alexa Fluor™ 594, Alexa Fluor™ 633, Alexa Fluor™ 647, Alexa Fluor™ 660, Alexa Fluor™ 680, 7-diethylaminocoumarin-3-carboxylic acid, Fluorescein, Oregon Green 488, Oregon Green 514, Tetramethylrhodamine, Rhodamine X, Texas Red dye, QSY 7, QSY33, Dabcyl, BODIPY® FL, BODIPY® 630/650, BODIPY® 6501665, BODIPYTMR-X®, BODIPY TR-X®, Dialkylaminocoumarin, Cy5.5, Cy5, Cy3.5, Cy3, DTPA(Eu3-)-AMCA and TTHA(Eu3-)AMCA.

Reporter molecules may be fluorescent organic dyes derivatized for attachment to the terminal 3′ or terminal 5′ ends of the probe via a linking moiety. Preferably, quencher molecules are also organic dyes, which may or may not be fluorescent, depending on the embodiment of the invention. The quencher molecule may be fluorescent. Generally, whether the quencher molecule is fluorescent or simply releases the transferred energy from the reporter by non-radiative decay, the absorption band of the quencher should substantially overlap the fluorescent emission band of the reporter molecule. Non-fluorescent quencher molecules that absorb energy from excited reporter molecules, but which do not release the energy radiatively, are referred to in the application as chromogenic molecules.

Exemplary reporter-quencher pairs may be selected from xanthene dyes, including fluoresceins, and rhodamine dyes. Many suitable forms of these compounds are widely available commercially with substituents on their phenyl moieties which may be used as the site for bonding or as the bonding functionality for attachment to an oligonucleotide. Another group of fluorescent compounds are the naphthylamines, having an amino group in the alpha or beta position. Included among such naphthylamino compounds are 1-dimethylaminonaphthyl-5-sulfonate, 1-anilino-8-naphthalene sulfonate and 2-p-touidinyl6-naphthalene sulfonate. Other dyes include 3-phenyl-7-isocyanatocoumarin, acridines, such as 9-isothiocyanatoacridine and acridine orange, N-(p-(2-benzoxazolyl)phenyl)maleimide, benzoxadiazoles, stilbenes, pyrenes, and the like. In one embodiment, reporter and quencher molecules are selected from fluorescein and rhodamine dyes.

There are many linking moieties and methodologies for attaching reporter or quencher molecules to the 5′ or 3′ termini of oligonucleotides, as exemplified by the following references: Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19: 3019 (1991) (3′ sulfhydryl); Giusti et al., PCR Methods and Applications, 2: 223-227 (1993) and Fung et al., U.S. Pat. No. 4,757,141 (5′ phosphoamino group via Aminolink™ II available from Applied Biosystems, Foster City, Calif.) Stabinsky, U.S. Pat. No. 4,739,044 (3′ aminoalkylphosphoryl group); Agrawal et al., Tetrahedron Letters, 31: 1543-1546 (1990) (attachment via phosphoramidate linkages); Sproat et al., Nucleic Acids Research, 15: 4837 (1987) (5′ mercapto group); Nelson et al., Nucleic Acids Research, 17: 7187-7194 (1989) (3′ amino group); and the like. Rhodamine and fluorescein dyes are also conveniently attached to the 5′ hydroxyl of an oligonucleotide at the conclusion of solid phase synthesis by way of dyes derivatized with a phosphoramidite moiety, e.g., Woo et al., U.S. Pat. No. 5,231,191; and Hobbs, Jr., U.S. Pat. No. 4,997,928.

An oligonucleotide can comprise a nucleic acid affinity tag. As used herein, “affinity tag” can refer to either a peptide affinity tag or a nucleic acid affinity tag. Affinity tag generally refer to a protein or nucleic acid sequence that may be bound to a molecule (e.g., bound by a small molecule, protein, covalent bond). An affinity tag may be a non-native sequence. A peptide affinity tag can comprise a peptide. A peptide affinity tag may be one that is able to be part of a split system (e.g., two inactive peptide fragments can combine together in trans to form an active affinity tag). A nucleic acid affinity tag can comprise a nucleic acid. A nucleic acid affinity tag may be a sequence that can selectively bind to a known nucleic acid sequence (e.g., through hybridization). A nucleic acid affinity tag may be a sequence that can selectively bind to a protein. An affinity tag may be fused to a native protein. An affinity tag may be fused to a nucleotide sequence. Sometimes, one, two, or a plurality of affinity tags may be fused to a native protein or nucleotide sequence. An affinity tag may be introduced into a oligonucleotide using methods of in vitro or in vivo transcription. Nucleic acid affinity tags can include, for example, a chemical tag, an RNA-binding protein binding sequence, a DNA-binding protein binding sequence, a sequence hybridizable to an affinity-tagged polynucleotide, a synthetic RNA aptamer, or a synthetic DNA aptamer. Examples of chemical nucleic acid affinity tags can include, but are not limited to, ribo-nucleotriphosphates containing biotin, fluorescent dyes, and digoxeginin. Examples of protein-binding nucleic acid affinity tags can include, but are not limited to, the MS2 binding sequence, the UlA binding sequence, stem-loop binding protein sequences, the boxB sequence, the eIF4A sequence, or any sequence recognized by an RNA binding protein. Examples of nucleic acid affinity-tagged oligonucleotides can include, but are not limited to, biotinylated oligonucleotides, 2,4-dinitrophenyl oligonucleotides, fluorescein oligonucleotides, and primary amine-conjugated oligonucleotides.

Oligonucleotides can have a modified nucleoside (i.e., base-sugar combination). The base portion of the nucleoside may be a heterocyclic base. The two most common classes of such heterocyclic bases are the purines and the pyrimidines. Nucleotides may be nucleosides that further include a phosphate group covalently linked to the sugar portion of the nucleoside. For those nucleosides that include a pentofuranosyl sugar, the phosphate group may be linked to the 2′, the 3′, or the 5′ hydroxyl moiety of the sugar. In forming oligonucleotides, the phosphate groups can covalently link adjacent nucleosides to one another to form a linear polymeric compound. In turn, the respective ends of this linear polymeric compound may be further joined to form a circular compound; however, linear compounds are generally suitable. In addition, linear compounds may have internal nucleotide base complementarity and may therefore fold in a manner as to produce a fully or partially double-stranded compound. Within oligonucleotides, the phosphate groups can commonly be referred to as forming the internucleoside backbone of the oligonucleotide. The linkage or backbone of the oligonucleotide may be a 3′ to 5′ phosphodiester linkage.

In some embodiments, the method further comprises a incorporating a modified DNA base into the nucleic acid molecules prior to oligonucleotide hybridization. Examples of modified DNA bases include, but are not limited to, methylated deoxy-cytosine-triphosphate (^{d5}mCTP), deoxyuridine triphosphate (dUTP), deoxyinosine triphosphate (ITP), ‘N-methyladenine, 8-oxo-guanine, +N-methylcytosine, 5-substituted pyrimidine and 7-substituted 7-deazapurine.

Incorporation of a modified DNA base may be useful in several embodiments of the present invention, including, but not limited to, to enable enrichment in the presence of C>T mutations. For example, one can first use primers to perform single primer extension, or PCR by using methylated dCTP (^{5m}C) in the place of standard dCTP, thereby converting a C>T mutation to a 5mC>T mutation. In some instances, UV-mediated cross-linking of a photo-activatable molecule (e.g., for CNVK) has a preference for 5mC instead of C. Accordingly, if C is replaced with ^{5m}C prior to applying the enrichment methods described herein, the methods described herein may enrich a mutant allele with a C>T mutation. In some embodiments, one may use dUTP in the place of dTTP, or dITP in the place of dCTP (I=Inosine). Incorporating any other base analogue that changes the relative reactivity between the photo-activatable molecule and a DNA base may be accomplished in the methods described herein in order to either lower or increase the reactivity of the oligonucleotide comprising a photo-activatable molecule with the target nucleic acid sequence or the non-target nucleic acid sequence.

In order to avoid excessive probe-to-probe binding in solution, while maximizing probe (oligonucleotide) to target binding, at least one, at least two, at least three, at least four, at least five, at least six, at least seven, at least eight, at least nine, at least ten etc. inosines can be added to the oligonucleotides disclosed herein. Inosines may be added to the 3′ end of the oligonucleotide. Inosine is known to pair with all 4 DNA nucleotides, A, T, C and G. In some embodiments, the inosines are consecutive to each other. In other embodiments, the inosines are not consecutive to each other optionally containing one two, three, four, or five or more DNA nucleotides (e.g., native DNA nucleotides) between each inosine.

In some embodiments, the Tm (melting temperature) of probe-binding to target will be significantly higher than the Tm of probe-probe binding in solution. For example, the Tm (melting temperature) of probe-binding (i.e., oligonucleotide-binding) to target can be at least 2, 3, 4, 5, 6, 7, 8, 9, 10, 11, 12, 13, 14, 15, 20, 25, or 30 degrees Celsius higher than the Tm of probe-probe binding (i.e., oligonucleotide-oligonucleotide binding) in solution.

When designing short oligonucleotides (e.g., oligonucleotides of less than 20, less than 15, less than 10, less than 5 nucleotides in length) with high melting temperatures (Tm) for use in the methods provided herein, selected nucleotides can be substituted by modified nucleotides that raise the Tm of the oligonucleotide in order to ensure hybridization of the oligonucleotide to the to the intended template, such as peptide nucleic acid, locked nucleic acid, ‘Super A, Super G, Super C, and/or Super T’, conjugation of intercalators that bind to the minor DNA groove and increase the Tm (e.g., minor groove binder (MGB probes)), or other synthetic base analogues that raise the Tm. For example, Super T™ (5-hydroxybutynl-2′-deoxyuridine) and Super GTM (8-aza-7-deazaguanosine) are non-limiting, representative examples of duplex-stabilizing modified bases that increase oligonucleotide Tm and are commercially available (such as by Integrated DNA Technologies; see idtdna.com).

In some embodiments, the method further comprises treating the nucleic acid molecules prior to oligonucleotide hybridization in order to convert un-methylated C to T, such treatment methods include, but are not limited to bisulfite (e.g., sodium bisulfite), methyl-seq, or APOBEC. Treatments capable of converting nucleic acid bases may be utilized in the methods described herein to either lower or increase reactivity of the oligonucleotide comprising a photo-activatable molecule with the target nucleic acid sequence or the non-target nucleic acid sequence.

In some embodiments, the nucleic acid amplification comprises isothermal amplification, such as recombinase-polymerase amplification (RPA), LAMP-isothermal amplification, or strand displacement amplification. Isothermal amplification methods provide detection of a nucleic acid target sequence in an exponential manner, but are not limited by thermal cycling. Isothermal methods do not rely on denaturing nucleic acids, instead it relies on a polymerase with strand-displacement activity to enable primer binding and initiation of the amplification reaction. Once the reaction is initiated, the polymerase must also separate the strand that is still annealed to the sequence of interest. Recombinase polymerase amplification (RPA) is a highly sensitive isothermal amplification technique, generally operating at 37-42° C., with minimal sample preparation and capable of amplifying as low as 1-10 nucleic acid target copies. It has been used to amplify targets such as RNA, miRNA, ssDNA and dsDNA. RPA amplification may be carried out in solution phase, solid phase as well as in a bridge amplification format. RPA has been successfully integrated with different detection strategies discussed herein, including, among others, real-time fluorescent detection amongst others. Loop-mediated isothermal amplification (LAMP), uses several primers that recognize several distinct regions of target nucleic acids for an amplification reaction. A strand-displacing DNA polymerase initiates synthesis and specially designed primers form “loop” structures to facilitate subsequent rounds of amplification through extension on the loops and additional annealing of primers.

The methods provided herein may include a step of depleting non-target nucleic acid molecules from the population of nucleic acid molecules. This method may comprise performing oligonucleotide hybridization with the population of nucleic acid molecules wherein the oligonucleotide is immobilized on a solid support, applying light to the population of hybridized oligonucleotide-nucleic acid molecules to induce a covalent cross-link between the photo-activatable molecule, and removing the solid supports comprising cross-linked nucleic acid molecules having non-target allele sequence from the population of nucleic acid molecules. The methods provided herein may include a step of depleting non-target nucleic acid molecules from the population of nucleic acid molecules. This method may comprise performing oligonucleotide hybridization with the population of nucleic acid molecules wherein the oligonucleotide is conjugated to a biotin moiety, applying light to the population of hybridized oligonucleotide-nucleic acid molecules to induce a covalent cross-link between the photo-activatable molecule, and removing the biotinylated cross-linked nucleic acid molecules having non-target allele sequence from the population of nucleic acid molecules. The step of depletion may be repeated from the population of nucleic acid molecules at least 2 times to at least 100 times and/or depleting non-target nucleic acid molecules from the population of nucleic acid molecules by at least 1-, 2-, 3-, 4-, 5-, 6-, 7-, 8-, 0-, 10-, 11-, 12-, 13-, 14-, 15-, 16-, 17-, 18-, 19-, 20-, 25-, 30-, 35-, 40-, 45-, 50-, 55-, 60-, 65-, 70-, 75-, 80-, 85-, 90-, 95-, 100-fold, or more, or any range in between, inclusive, such as 10-fold to 50-fold.

As described herein, the oligonucleotides comprise photo-activatable molecules that are capable of cross-linking with target or non-target nucleic acid sequences when exposed to light (e.g., UV or visible light). The light may be applied before, concurrently with, or after any step of PCR thermocycling. The light may be applied more than once, optionally wherein the light is applied more than once before, concurrently with, or after any step of PCR thermocycling.

In some embodiments, the nucleic acid amplification oligonucleotides and primers are used in a real-time nucleic acid amplification method. Since post-amplification amplicon detection is both laborious and time consuming, real-time methods have been developed to monitor amplification during the amplification and are well-known in the art. These methods typically employ fluorescently labeled probes that bind to the newly synthesized DNA or dyes whose fluorescence emission is increased when intercalated into double stranded DNA. Real-time detection methodologies are applicable to PCR detection of target nucleic acid sequences in genomic DNA or genomic RNA.

Quantitative PCR (qPCR) is used to amplify and simultaneously quantify one or more targeted nucleic acid templates. The quantity may be either an absolute number of copies or a relative amount when normalized to a known DNA input (e.g., an internal or external control) or additional normalizing genes (e.g., housekeeping gene such as beta-actin). Three common methods for qPCR detection are: (1) non-specific fluorescent dyes that intercalate with double-stranded DNA, (2) sequence-specific probe(s) labeled with a fluorescent reporter which permits detection only after hybridization of the probe (e.g., molecular beacon), and (3) sequence-specific probes that are hydrolyzed by a PCR polymerase, such as TaqMan® probes. qPCR is compatible both with quantitative competitive PCR, where internal competitor for each target sequence is used for normalization, and with quantitative comparative PCR using a normalization gene contained within the sample, or a housekeeping gene for RT-PCR (see Held et al. (1996) Genome Research 6:986-994).

Nucleic acid amplification may be performed using well-known and commercially available instruments. For example, a qPCR reaction may be performed using an Applied Biosystems® 7300 Real Time PCR System, which was used in the examples described below. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

In some embodiments, detected nucleic acid amplification product amounts of interest are compared with such amounts of a reference nucleic acid amplification product. For example, a reference may be another experimental gene of interest or a control that is expected to stay constant in the sample. For the reference, a housekeeping gene (e.g., a gene that is required for the maintenance of basic cellular function) may be used that is expected to stay constant in a sample. A housekeeping gene that may be used as reference in the methods described herein can include a gene that encodes a transcription factor, a transcription repressor, an RNA splicing gene, a translation factor, tRNA synthetase, RNA binding protein, ribosomal protein, RNA polymerase, protein processing protein, heat shock protein, histone, cell cycle regulator, apoptosis regulator, oncogene, DNA repair/replication gene, carbohydrate metabolism regulator, citric acid cycle regulator, lipid metabolism regulator, amino acid metabolism regulator, nucleotide synthesis regulator, NADH dehydrogenase, cytochrome C oxidase, ATPase, mitochondrial protein, lysosomal protein, proteosomal protein, ribonuclease, oxidase/reductase, cytoskeletal protein, cell adhesion protein, channel or transporter, receptor, kinase, growth factor, tissue necrosis factor, etc. Specific examples of housekeeping genes that may be used in the methods described include, e.g., HSP90, ACTB, UBC and TUBA1B. In some embodiments, the entire translatome is globally decreased or increased such that the term “housekeeping gene” refers to a gene whose trend in change is broadly representative of the majority of the translatome change.

In addition to relative quantitation, the methods of the present invention can incorporate comparisons of the amounts of target nucleic acids in an absolute quantitation mode, such as by comparing the amounts of target nucleic acids fixed standards either in separate reactions or added artificially to the sample in earlier steps.

For example, many nucleic acid hybridization-based, sequencing-based, and/or amplification-based assays for detecting and analyzing nucleic acids, such as Southern blotting, Northern blotting, comparative genomic hybridization (CGH), chromosomal microarray analysis (CMA), expression profiling, DNA microarray, high-density oligonucleotide microarray, whole-genome RNA expression array, digital PCR (dPCR), reverse transcription PCR, ligation chain reaction (sometimes referred to as oligonucleotide ligase amplification OLA), cycling probe technology (CPT), strand displacement assay (SDA), transcription mediated amplification (TMA), nucleic acid sequence based amplification (NASBA), rolling circle amplification (RCA) (for circularized fragments), invasive cleavage assays, nCounter Analysis (Nanostring technology), genome sequencing, de novo sequencing, pyrosequencing, polony sequencing, copy number variation (CNV) analysis sequencing, small nucleotide polymorphism (SNP) analysis, whole exome sequencing, in situ hybridization, either DNA or RNA fluorescent in situ hybridization (FISH), chromogenic in-situ hybridization (CISH), RNA sequencing, and epigenetic profiling, such as methylation pattern sequencing, phosphorylation pattern sequencing, and the like, may be used.

Subsequent to the enrichment methods described herein, the mutation-enriched (or methylation-enriched) sequences may be screened via any currently available method for identifying mutations, including, but not limited to Sanger Sequencing, high resolution melting (HRM), single strand confirmation polymorphism (SSCP), next generation sequencing, MALDI-TOF, high resolution melting, single molecule sequencing, third generation sequencing, or any other method involving high-throughput sequencing.

So-called “next-generation” sequencing techniques that may be amenable to performing large numbers of sequencing reactions in parallel may be performed. Such techniques include pyrosequencing, nanopore sequencing, single base extension using reversible terminators, ligation-based sequencing, single molecule sequencing techniques, massively parallel signature sequencing (MPSS) and the like, as described in, for example, U.S. Pat. Nos. 7,057,056; 5,763,594; 6,613,513; 6,841,128; and 6,828,100; and PCT Published Application Nos. WO 07/121,489 A2 and WO 06/084132 A2.

Many technologies using or detecting nucleic acids may also be adapted for arrays, which are sensitive to size variations because a multitude of individual reactions occur in densely packed locations. As used herein, an “array,” includes any one-dimensional, two-dimensional or substantially two-dimensional (as well as a three-dimensional) arrangement of addressable regions (i.e., features, e.g., in the form of spots) bearing nucleic acids, particularly oligonucleotides or synthetic mimetics thereof (i.e., the oligonucleotides defined above), and the like. Where the arrays are arrays of nucleic acids, the nucleic acids may be adsorbed, physisorbed, chemisorbed, or covalently attached to the arrays at any point or points along the nucleic acid chain. Array-based assays are well-known in the art and include, for example, comparative genomic hybridization (CGH) and array-based comparative genomic hybridization (aCGH).

### C. Kits

The disclosure herein also provides for a kit format which comprises a package unit having one or more reagents for the generation of nucleic acid fragments, enrichment methods and/or amplification methods as described herein. The kit may also contain one or more of the following items: buffers, instructions, and positive or negative controls. Kits may include containers of reagents mixed together in suitable proportions for performing the methods described herein. Reagent containers preferably contain reagents in unit quantities that obviate measuring steps when performing the subject methods.

Kits may also contain reagents for quantitative real-time PCR including, but not limited to, a thermostable nucleic acid polymerase, buffers, fluorescent detection reagents, and nucleic acid amplification primers to amplify the real-time PCR products of interest and to allow for the quantitative detection of the target nucleic acid sequence according to the methodology described herein.

In another embodiment, the kit reagents further comprised reagents for the extraction of nucleic acid molecules of interest, such as those from a biological sample. Kit reagents may also include reagents for PCR analysis where applicable.

### Exemplification

This invention is further illustrated by the following examples, which should not be construed as limiting.

### Example 1: Materials and Methods for Examples 2-7

### Genomic DNA and Clinical Samples

Human male genomic DNA (Promega Corporation) was used as wild type (WT) DNA. Genomic DNA from different cell lines (PFSK, A549, SW480, H2009 or LOVO) was spiked into human male genome DNA (HMC) to generate serial dilutions of mutated genomic DNA. Snap-frozen lung tumor specimens were obtained from the Massachusetts General Hospital Tumor Bank and were used following approval from the Internal Review Board of the Dana Farber Cancer Institute. The DNAeasy™ Blood & Tissue Kit (Qiagen) was used to isolate genomic DNA from tumor samples. A Qubit 3.0 fluorometer and dsDNA HS assay (Thermo Fisher Scientific) were applied to measure the DNA concentration.

### Setup for Uniform UV Irradiation of Samples During Open-Lid PCR

To enable UV-irradiation during PCR cycling, an open-lid PCR approach was used, with a UV light source replacing the PCR machine lid (FIG. 5A). Open-lid PCR was applied on an Eppendorf Mastercycler™ Nexus GX2 Thermal cycler. The lid of this cycler may be opened anytime during PCR without interrupting the PCR program. A UV lamp assembly with an average wavelength ˜365 nm wavelength (realUV™ LED Flood Light, Waveform Lighting Inc) was placed in a pre-defined, reproducible position in direct contact with a 96-well plate to provide consistent UV irradiation of PCR wells between experiments. During irradiation, the UV lamp was covered to prevent UV exposure to operators. In this geometry the average distance between the actual light source and PCR samples was ˜ 4 cm, providing an approximate irradiance of 36.5 mW/cm² to the samples as estimated from the manufacturer-provided specification.

To define the fraction of the 96-well plate receiving uniform UV irradiation among wells, control experiments were performed using cross-linking of DNA with the intercalator psoralen, that cross-links DNA strands upon irradiation with 365 nm UV light. Upon cross-linking, DNA is rendered un-amplifiable, thereby the delay in real-time PCR amplification following UV irradiation is a measure of DNA cross-linking and UV exposure. Real time PCR was used to quantify the amount of DNA cross-linking and wells that demonstrated less than 1 threshold cycle difference of each other during a subsequent real time PCR reaction were considered as receiving uniform UV irradiation (FIG. 5B). In the absence of UV irradiation, real-time PCR thresholds were approximately ˜5 cycles earlier than those receiving UV irradiation. Using this approach, it was determined that under the geometry applied, 16 wells (rows D-G, columns 1-4) were receiving uniform UV irradiation and were used for subsequent UVME-PCR experiments.

### UV-Mediated Cross-Linking Minor-Allele Enrichment (UVME) PCR Reaction

Serial dilutions of mutated genomic DNA from A549, SW480, H2009, LOVO or PFSK into wild type DNA were used for validation of UVME-PCR. Primers and CNVK-modified probes were purchased from Integrated DNA Technologies and GeneLink™, respectively, and their sequences are listed on Table 1 below.

TABLE 1
Primers and CNVK-modified probes

| Primer ID | Sequences |
|---|---|
| TP53 F1 | 5′-CCTATCCTGAGT |
| | AGTGGTAATCT-3′ |
| TP53 81 | 5′-TTACCTCGCTTA |
| | GTGCTC-3′ |
| KRAS F1 | 5′-CATTATTTTTAT |
| | TATAAGGCCTGC-3′ |
| KRAS R1 | 5′-CAAAATGATTCT |
| | GAATTAGCTGT-3′ |
| Probe ID | Sequences |
| TP53 target-specific | 5′-GGC ACAAACA^{CNV} |
| CNVK-modified probe | KG CAC-3 |
| TP53 common CNVK- | 5′-CAGAGGAAGAGA |
| modified probe | A^{CNV}CTCCG/phos-3′ |
| KRAS target-specific | 5′-CCTA^{CNV}KGCCAC |
| CNVX-modified probe | CAGCTCCA/phos-3′ |
| KRAS common CNVK- | 5′-TGAAAATGA^{CNV}K |
| modified probe | TGAATATAAACTTGT |
| | GG/phos-3′ |

For experiments using serial dilutions of DNA with KRAS-mutations, DNA was added in a final of 25 μl PCR reaction containing 1.2 μM CNVK target-specific probes and CNVK common probes, 0.8X LC green (BioFire Diagnostics), 1X AmpliTaqGold buffer, 0.8 mM dNTP, 0.2 mM forward and reverse primers, 2 mM MgCl₂, 2 μl GC enhancer, 0.06 μl Phusion™ polymerase (Thermo Fisher Scientific) and 0.125 μl AmpiTaq™ polymerase (Thermo Fisher Scientific). Fifty-μl mineral oil (Sigma-Aldrich) was overlaid on the PCR master mix to prevent evaporation during open lid PCR. The tubes were vortexed and spun-down before placing in the PCR well plates. The PCR reaction was performed with an initial activation for 2 min at 95° C. followed by 10 cycles of closed lid PCR (95° C. for 30 see, 57° C. for 30 see and 72° C. for 10 see); then, without interrupting the program, the Eppendorf machine lid was opened, the UV source and cover were attached, and the cycling continued with 50 cycles of open lid PCR (95° C. for 30 see, 57° C. for 30 see and 72° C. for 10 see). UV irradiation was applied for 10 see, after the initial 10 see at 57° C. during each cycle of the open lid PCR stage. The UVME-PCR products were placed on ice or stored in-20° C. before dilution for downstream assays.

To combine UVME-PCR reactions with temperature-tolerant fast COLD-PCR (UVME-TT-fast-COLD-PCR) for KRAS mutations, the closed-lid pre-amplification stage of the reaction was increased to a total of 20 cycles and conducted as follows: an initial denaturation for 1 min at 95° C. following by 9 cycles of closed lid PCR (81.2° C. for 30 see, 57° C. for 30 see and 72° C. for 10 see), 11 cycles of closed lid PCR (82.2° C. for 30 see, 57° C. for 30 see and 72° C. for 10 see); followed by 50 cycles of open lid PCR with UV irradiation as described above.

For experiments using serial dilutions of DNA with p53 mutations, similar protocols as with KRAS were used, with minor differences; Phusion™ polymerase was not included in the reaction and 2.4 μM CNVK-modified probes were used in the UVME-PCR reaction. The PCR reaction was conducted with initial activation for AmpliTaq polymerase at 95° C. for 10 min followed by 20 cycles closed lid PCR (95° C. for 30 see, 60° C. for 30 see and 72° C. for 10 see), 50 cycles of open lid PCR (95° C. for 30 see, 53° C. for 30 see, and 72° C. for 30 see), and 72° C. for 7 min.). UV irradiation was applied for 10 see, after the initial 10 see at 57° C. during each cycle of the open lid PCR stage. UVME experiments were repeated in triplicate independent experiments.

### Direct Sanger Sequencing

The UVME-PCR products were submitted to Genewiz, Inc for Sanger sequencing using target specific sequencing primers (Table 2). BioEdit Sequence Alignment Editor (Bioedit Ltd) was applied to illustrate and capture the signaling peaks.

TABLE 2
Sequencing Primers

| Primer ID | Sequences |
|---|---|
| TP53 sequencing | 5′-CGGAGATTCTCTTCCTCT-3′ |
| primer | |
| KRAS sequencing | 5′-TTTTTTTTTTTTTTTTTTT |
| primer | TTTTTTTTTTTCATTATTTTTA |
| | TTATAAGGCCTGC-3′ |

TaqMan® real time genotyping assays

UVME-PCR and UVME-TT-fast-COLD-PCR reactions were followed by regular TaqMan® genotyping (40) for detection and quantification of mutations. Sequences of primers and probes used for TaqMan® assays are listed on Table 3. The UVME-PCR or UVME-TT-fast-COLD-PCR products were diluted (1:20,000) in water. One-ul diluted DNA was applied into 12.5 μl volume TaqMan® genotyping reaction containing 1X TaqMan® genotyping master mix, 260 nM probes and 900 nM forward and reverse primers. The TaqMan® reaction was conducted on a CFX Connect™ real-time PCR (Bio-Rad) with 10 min initial activation at 95° C. following by 50 cycles of 30 see denaturation at 95° C. and 1 min annealing/extension at 60° C. The mutation allelic frequency (mutation abundance) was calculated by correlating a standard curve created from serial dilutions of mutated genomic DNA.

TABLE 3
Sequences of primers and probes
for TaqMan® assays

| Primer ID | Sequences |
|---|---|
| TP53 F2 | 5′-TGGTAATCTACT |
| | GGGACG-3′ |
| TP53 R2 | 5′-CGGAGATTCTCT |
| | TCCTCT-3′ |
| KRAS F2 | 5′-TGAAAATGACTG |
| | AATATAAACTTGTG- |
| | 3′ |
| KAS R2 | 5′-CTGAATTAGCTG |
| | TATCGTCAAG-3′ |
| Probe ID | Sequences |
| TP53-WT | 5′-HEX-TTTGAGGT |
| | GCGTGTTTGTGCC- |
| | BHQ1-3′ |
| TP53-Mut | 5′-FAM-TGCGTGTT |
| | GGTGCCTGTC-BHQ1 |
| | -3′ |
| KRAS-WT | 5′-FAM-TGGAGCTG |
| | GTGGCGTAG-BHQ1- |
| | 3′ |
| KRAS-G12A-Mut | 5′-HEX-TGGAGCTG |
| | CTGGCGTAG-BHQ1- |
| | 3′ |
| KRAS-G12S-Mut | 5′-HEX-TGGAGCTA |
| | GTGGCGTAGG-BHQ1 |
| | -3′ |
| KRAS-G12V-Mut | 5′-HEX-TGGAGCTG |
| | TTGGCGTAGG-BHQ1 |
| | -3′ |
| KRAS-G13D-Mut | 5′-HEX-TGGAGCTG |
| | GTGACGTAGG-SHQ1 |
| | -3′ |
| KRAS-G12D-Mut | 5′-HEX-AGCTGATG |
| | GCGTAGGCA-BHQ1- |
| | 3′ |

Droplet Digital PCR (ddPCR)

UVME-PCR and UVME-TT-fast-COLD-PCR reactions were alternatively followed by droplet digital PCR for detection and quantification of mutations. Sequence of primers and probes for p53 mutations are listed on Table 4. Droplet amplifications were performed in a 20 μl volume containing 1X ddPCR supermix for probes (Bio-rad), 250 μM probes (for PFSK), 900 nM forward and reverse primers (for PFSK), and 1 μl of diluted UVME-PCR product (1:1M dilution in water) or UVME-fast-COLD-PCR product (1:20000 dilution in water). For KRAS mutations, 1 μl of diluted UVME-PCR product (1:1M dilution in water) or UVME-TT-fast-COLD-PCR product (1:20000 dilution in water) was employed in a 20 μl volume reaction containing 1 μl of 20X ddPCR gene expression assays (Bio-rad) and 1X ddPCR supermix for probes (Bio-rad). The master mix was then applied on DG8TM droplet generator cartridges (Bio-rad) containing 70 μl of droplet generation oil (Bio-rad) for droplet generation. The droplets were then transferred to a 96-well plate and sealed with PX1 PCR plate sealer (Bio-rad) for 5 see at 180° C. The thermal cycling was performed on an Eppendrof Mastercycler (Eppendorf) with an initial denaturation step at 95° C. for 10 min following by 40 cycles of 30 see denaturation at 94° C., 60 see annealing at 55° C. and a final step at 98° C. for 10 min. The plate was then transferred to QX200 droplet reader (Bio-rad) for droplets reading. Quantasoft (Bio-rad) was applied to analyze the positive droplets from FAM and HEX channels. Poisson distribution model with 95% confidence level was applied to calculate the percentage of fractional abundance. Additional details regarding ddPCR may be found at least in Lindner L, Cayrou P, Jacquot S., Birling M. C., Herault Y., Pavlovic G. (2021) Reliable and robust droplet digital PCR (ddPCR) and RT-ddPCR protocols for mouse studies. Methods. 191, 95-106 and Olmedillas-López S., García-Arranz M., García-Olmo D. (2017) Current and Emerging Applications of Droplet Digital PCR in Oncology. Mol Diagn Ther., 21(5),493-510.

TABLE 4
Sequences of primers and
probes for ddPCR assays

| Primer ID | Sequences |
|---|---|
| TP53 F2 | 5′-TGGTAATCTACT |
| | GGGACG-3′ |
| TP53 R2 | 5′-CGGAGATTCTCT |
| | TCCTCT-3′ |
| Probe ID | Sequences |
| TP53-WT | 5′-HEX-TTTGAGGT |
| | GCGTGTTTGTGCC-B |
| | HQ1-3′ |
| TP53-Mut | 5′-FAM-TGCGTGTT |
| | GGTGCCTGTC-BHQ1 |
| | -3′ |

### High Resolution Melting (HRM) Analysis

Eight-μl of UVME-PCR or UVME-fast-COLD-PCR products were transferred to a 96 well plate. Twenty-five-μl of oil were then overlaid to prevent evaporation. The 96 well plate was sealed with PX1 PCR plate sealer (Bio-rad) for 3 see at 180° C. and briefly centrifuged. The plate was run on LightScanner R system (IdahoTechnology) and the Lightscanner software was applied for analysis. HRM is based on detecting small differences in PCR dissociation curves. HRM may employ the use of dsDNA-binding dyes used in conjunction with real-time PCR instrumentation that has a temperature ramp control and advanced data capture capabilities. Data may be analyzed and manipulated using software designed specifically for HRM analysis.

### Statistical Analysis

PRISM 9 (Graphpad) was applied on statistical analyses. Statistical significance was determined via the un-paired student T test. Samples marked with “*” represent those with values significantly different (P<0.05) from the value of corresponding wild type controls run in parallel. Otherwise samples are marked as “ns” (P>0.05, non-significant). Two-three independent experiments were performed for each data point.

### Example 2: An Embodiment of the Methods Provided Herein for Pyrimidine enrichment methods

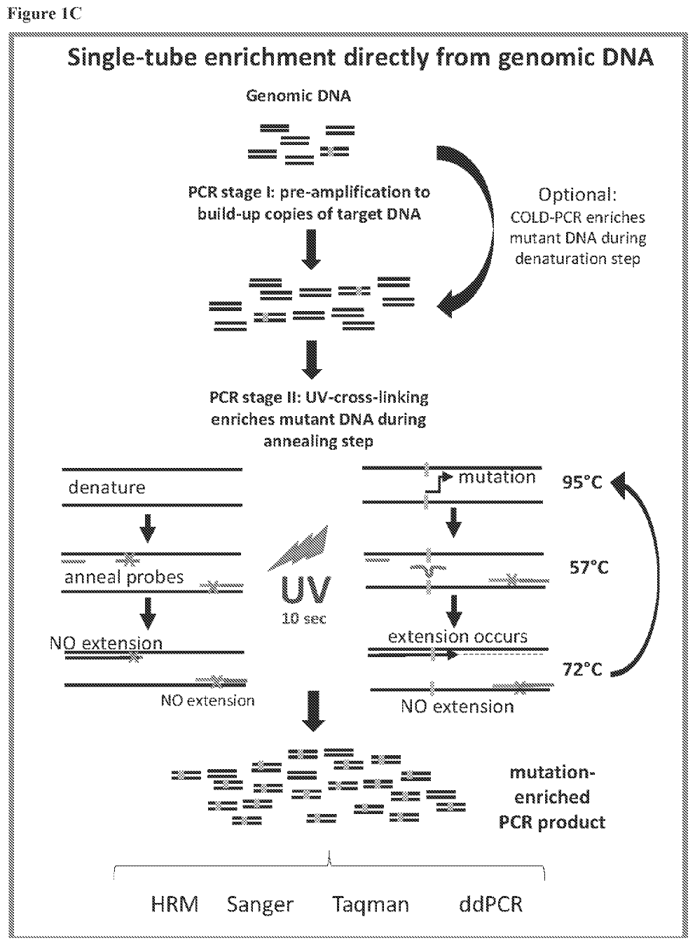

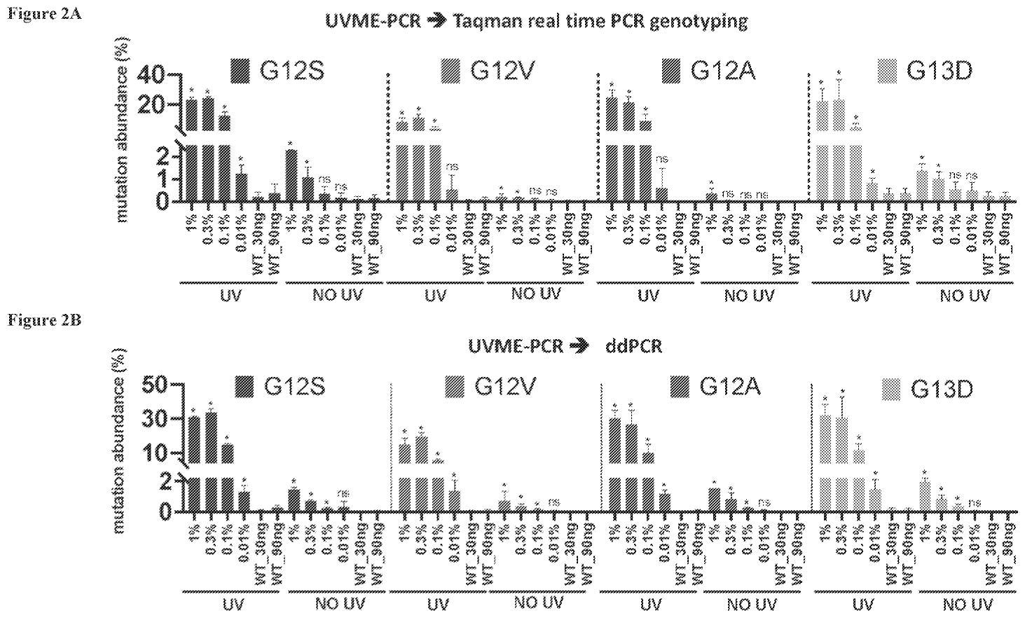

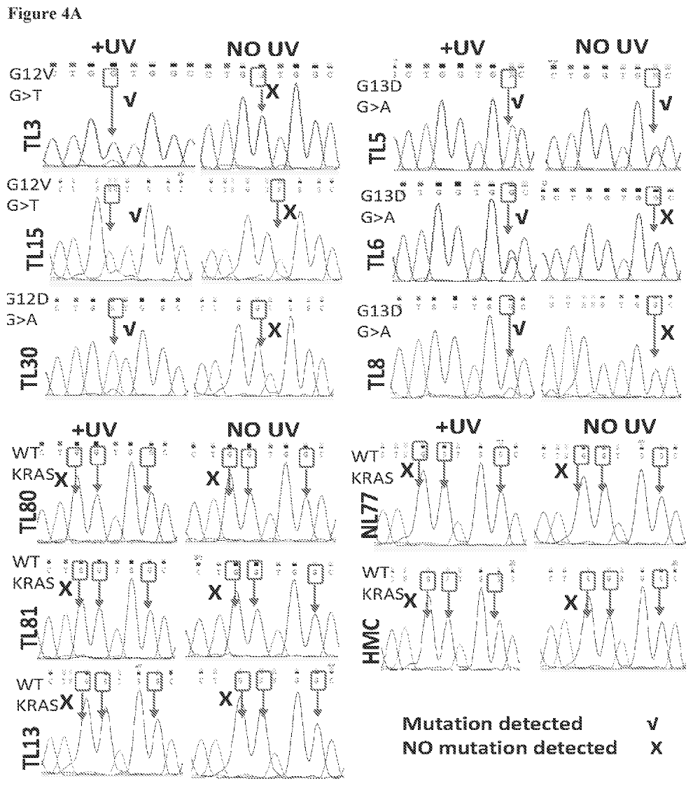

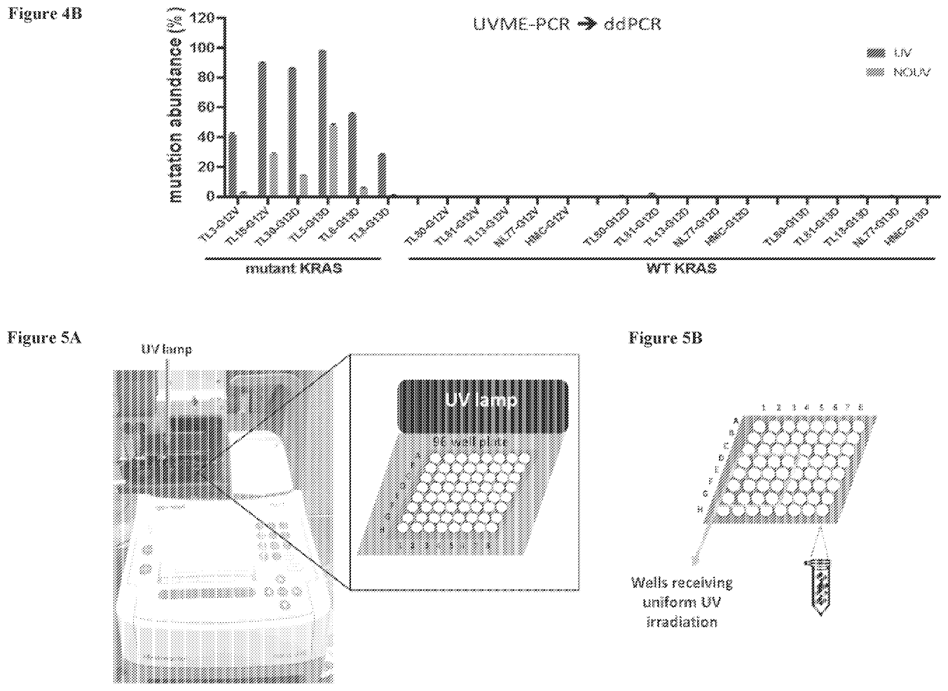

An exemplary photo-activatable molecule described herein, CNVK, has highest reactivity with T, modest reactivity with C and no reactivity with purines like A or G. CNVK (3-cyanovinylcarbazole) is a molecule that may be photo-activated by UV irradiation at 365 nm wavelength. When a CNVK-containing nucleoside is incorporated into oligonucleotides that are then made to hybridize to nucleic acid molecules, it enables UV-enabled rapid cross-linking with thymine or cytosine at −1 position of the opposite DNA strand. In the present disclosure, one example of oligonucleotide probes matching the wild type sequence and incorporating a 3-cyanovinylcarbazole nucleoside (CNVK) modification at the right sequence position are employed for selective crosslinking of wild type DNA during UVME to enable enrichment of specific mutations before, during or after a PCR reaction. FIG. 4 demonstrates a representative application of the methods disclosed herein, using CNVK-containing oligonucleotide probes to selectively cross-link a wild type BRAF DNA target, in order to enrich a clinically relevant V600E mutation in BRAF gene. CNVK (which may be referred to as “K”)-containing oligonucleotides, such as those comprising 5-200 nucleotides, are hybridized to a BRAF exon 15 target. Hybridization brings K opposite a T (at −1 position) in the WT BRAF sequence. In contrast, if the T>A mutation is present, there is no T opposite K. Upon UV irradiation, K is cross-linked to the wild type (WT) sequence but not to the mutated sequence, since K can only cross-link to T or C in the opposite strand. Upon subsequent amplification of the BRAF DNA target from genomic DNA, the cross-linked strand of wild type BRAF does not amplify, thereby leading to enrichment of mutated sequences over wild type. FIG. 2 shows that if UV is applied either once before PCR, or alternatively, during the annealing stage of each PCR cycle, the CNVK-containing oligonucleotides crosslink preferentially to the wild type DNA strand that they target. Following cross-linking the sense strands cannot be copied by the polymerase during a subsequent PCR amplification. As a result, the wild type DNA amplifies linearly (only bottom strand gets copied), while the mutated DNA amplifies exponentially. Additionally, since the mutated sequence contains 2 mismatches with probe while the wild type sequence only contains one mismatch (i.e., the base opposite K), there is reduced oligonucleotide probe hybridization to target for mutated BRAF, thereby increasing the mutation enrichment even further.

UV irradiation may be applied to a sample before PCR, during PCR, or at a post-PCR step. If applied post-PCR, a second nested PCR must be conducted to enrich the mutation-containing DNA. Therefore, the UV crosslinking-mediated inhibition of the WT DNA target can take place at any point during the process and prior to an endpoint detection method applied to identify the mutation (sequencing, ddPCR, etc).

PCR amplification used may be any form of PCR previously described, such as COLD-PCR, touch-down PCR, arbitrarily-primed PCR (AP-PCR). The UV crosslinking may be applied at any stage during the PCR cycling, e.g., either in the annealing step, or in the denaturation step, or at an intermediate temperature step. It is noteworthy that the amplification step that selectively amplifies the mutated version of the target of interest is not restricted to PCR but may be isothermal-amplification such as RPA (recombinase-polymerase amplification), LAMP-isothermal amplification, or amplification via strand displacement using phi29 or BST polymerases, or any other known form of isothermal amplification. UV-induced crosslinking may be conducted either before isothermal amplification or during isothermal amplification to result to a product with enriched mutated target sequences.

PCR-based detection of low-level DNA mutations comprises a frequently-used approach to reveal recurrent, hotspot genetic changes of clinical relevance to cancer, pre-natal diagnostics, transplantation, or infectious diseases.

However, the high-excess wild type (WT) alleles poses complex requirements that call for development of improved PCR-methodologies. Here we introduce UV-mediated Cross-linking Minor-allele Enrichment (UVME-PCR), a novel approach that incorporates ultraviolet irradiation (UV ˜365 nm) DNA cross-linking during PCR. Oligonucleotide probes matching the sense strand wild type target sequence and incorporating a UV sensitive 3-cyanovinylcarbazole nucleoside (CNVK) modification are employed for crosslinking wild type DNA. Mismatches formed with mutated alleles reduce DNA binding and UV-mediated crosslinking and favor mutated-DNA amplification. UV may be applied at any stage during PCR to selectively block wild type DNA amplification and enable identification of traces of mutated alleles. This enables a single-tube PCR reaction directly from genomic DNA combining optimal pre-amplification of mutated alleles which then switches to UV-mediated mutation-enrichment-based DNA target amplification. UVME-PCR reactions directly from genomic DNA enable >100-fold enrichment of mutated KRAS and p53 alleles which may be screened directly via Sanger sequencing, high-resolution-melting, TaqMan®-genotyping or digital-PCR, resulting to detection of mutation-allelic-frequencies of 0.1%-0.001% depending on endpoint-detection method. UV mediated mutation enrichment provides new potential for PCR-based mutation enrichment in diverse clinical samples.

Identification of DNA mutations at low allelic frequencies within an excess of wild type alleles is an essential requirement for analysis of clinical samples on several occasions, including cancer, organ transplantation, pre-natal diagnostics and infectious diseases. More details regarding contexts in which the methods of the present application may be applied may be found at least in Kobayashi, S., Boggon, T. J., Dayaram, T., Janne, P.A., Kocher, O., Meyerson, M., Johnson, B. E., Eck, M. J., Tenen, D. G. and Halmos, B. (2005) EGFR mutation and resistance of non-small-cell lung cancer to gefitinib. N Engl J Med, 352, 786-792; Hoffmann, C., Minkah, N., Leipzig, J., Wang, G., Arens, M. Q., Tebas, P. and Bushman, F. D. (2007) DNA bar coding and pyrosequencing to identify rare HIV drug resistance mutations. Nucleic Acids Res, 35, e91; Lo, Y. M., Corbetta, N., Chamberlain, P. F., Rai, V., Sargent, I. L., Redman, C. W. and Wainscoat, J. S. (1997) Presence of fetal DNA in maternal plasma and serum. Lancet, 350, 485-487; Snyder, T. M., Khush, K. K., Valantine, H. A. and Quake, S.R. (2011) Universal noninvasive detection of solid organ transplant rejection. Proc Natl Acad Sci USA, 108, 6229-6234; Dong, S. M., Traverso, G., Johnson, C., Geng, L., Favis, R., Boynton, K., Hibi, K., Goodman, S. N., D′Allessio, M., Paty, P. et al. (2001) Detecting colorectal cancer in stool with the use of multiple genetic targets. J Natl Cancer Inst, 93, 858-865; and Galbiati, S., Brisci, A., Lalatta, F., Seia, M., Makrigiorgos, G.M., Ferrari, M. and Cremonesi, L. (2011) Full COLD-PCR protocol for noninvasive prenatal diagnosis of genetic diseases. (lin Chem, 57, 136-138.). For example in liquid biopsies of cancer using circulating-free DNA, low-levels of somatic mutations or aberrant methylation serve as biomarkers for early detection, minimal residual disease detection or tumor response to treatment, yet the high excess of circulating wild type DNA often restricts the opportunities for diagnosis and treatment. More details describing exemplary clinical contexts in which the methods described herein may be applied may be found at least in Diehl, F., Schmidt, K., Choti, M.A., Romans, K., Goodman, S., Li, M., Thornton, K., Agrawal, N., Sokoll, L., Szabo, S. A. et al. (2008) Circulating mutant DNA to assess tumor dynamics. Nat Med, 14, 985-990; Thierry, A. R., Mouliere, F., El Messaoudi, S., Mollevi, C., Lopez-Crapez, E., Rolet, F., Gillet, B., Gongora, C., Dechelotte, P., Robert, B. et al. (2014) Clinical validation of the detection of KRAS and BRAF mutations from circulating tumor DNA. Nat Med, 20, 430-435; Newman, A. M., Bratman, S. V., To, J., Wynne, J. F., Eclov, N. C., Modlin, L. A., Liu, C. L., Neal, J. W., Wakelee, H. A., Merritt, R. E. et al. (2014) An ultrasensitive method for quantitating circulating tumor DNA with broad patient coverage. Nat Med, 20, 548-554; Bettegowda, C., Sausen, M., Leary, R. J., Kinde, I., Wang, Y., Agrawal, N., Bartlett, B. R., Wang, H., Luber, B., Alani, R. M. et al. (2014) Detection of circulating tumor DNA in early—and late-stage human malignancies. Sci Transl Med, 6, 224ra224; Diehl, F., Li, M., Dressman, D., He, Y., Shen, D., Szabo, S., Diaz, L. A., Jr., Goodman, S. N., David, K. A., Juhl, H. et al. (2005) Detection and quantification of mutations in the plasma of patients with colorectal tumors. Proc Natl Acad Sci USA, 102, 16368-16373). Liu, M. C., Oxnard, G. R., Klein, E. A., Swanton, C., Seiden, M. V., Liu, M.C., Oxnard, G. R., Klein, E. A., Smith, D., Richards, D. et al. (2020) Sensitive and specific multi-cancer detection and localization using methylation signatures in cell-free DNA. Annals of Oncology, 31, 745-759; Cohen, J. D., Li, L., Wang, Y., Thoburn, C., Afsari, B., Danilova, L., Douville, C., Javed, A. A., Wong, F., Mattox, A. et al. (2018) Detection and localization of surgically resectable cancers with a multi-analyte blood test. Science, 359, 926-930; Parsons, H. A., Rhoades, J., Reed, S. C., Gydush, G., Ram, P., Exman, P., Xiong, K., Lo, C.C., Li, T., Fleharty, M. et al. (2020) Sensitive Detection of Minimal Residual Disease in Patients Treated for Early-Stage Breast Cancer. Clin Cancer Res.; Kimura, T., Holland, W. S., Kawaguchi, T., Williamson, S. K., Chansky, K., Crowley, J. J., Doroshow, J. H., Lenz, H.J., Gandara, D. R. and Gumerlock, P. H. (2004) Mutant DNA in plasma of lung cancer patients: potential for monitoring response to therapy. Ann N Y Acad Sci, 1022, 55-60; Misale, S., Yaeger, R., Hobor, S., Scala, E., Janakiraman, M., Liska, D., Valtorta, E., Schiavo, R., Buscarino, M., Siravegna, G. et al. (2012) Emergence of KRAS mutations and acquired resistance to anti-EGFR therapy in colorectal cancer. Nature, 486, 532-536; or Chabon, J. J., Hamilton, E. G., Kurtz, D. M., Esfahani, M. S., Moding, E. J., Stehr, H., Schroers-Martin, J., Nabet, B. Y., Chen, B., Chaudhuri, A. A. et al. (2020) Integrating genomic features for non-invasive early lung cancer detection. Nature, 580, 245-251.

Next-generation sequencing technologies (NGS) combined with incorporation of molecular barcodes may be able to provide enumeration of rare mutations on an exome or genome-wide levels (more details regarding NGS may be found at least in Newman, A.M., Lovejoy, A. F., Klass, D. M., Kurtz, D. M., Chabon, J. J., Scherer, F., Stehr, H., Liu, C. L., Bratman, S. V., Say, C. et al. (2016) Integrated digital error suppression for improved detection of circulating tumor DNA. Nat Biotechnol, 34, 547-555; Wan, J. C. M., Heider, K., Gale, D., Murphy, S., Fisher, E., Mouliere, F., Ruiz-Valdepenas, A., Santonja, A., Morris, J., Chandrananda, D. et al. (2020) ctDNA monitoring using patient-specific sequencing and integration of variant reads. Sci Transl Med, 12; McDonald, B. R., Contente-Cuomo, T., Sammut, S. -J., Odenheimer-Bergman, A., Ernst, B., Perdigones, N., Chin, S. -F., Farooq, M., Mejia, R., Cronin, P. A. et al. (2019) Personalized circulating tumor DNA analysis to detect residual disease after neoadjuvant therapy in breast cancer. Science Translational Medicine, 11, eaax7392; Kinde, I., Wu, J., Papadopoulos, N., Kinzler, K. W. and Vogelstein, B. (2011) Detection and quantification of rare mutations with massively parallel sequencing. Proc Natl Acad Sci USA, 108, 9530-9535; or Schmitt, M. W., Kennedy, S.R., Salk, J. J., Fox, E. J., Hiatt, J. B. and Loeb, L.A. (2012) Detection of ultra-rare mutations by next-generation sequencing. Proc Natl Acad Sci USA, 109, 14508-14513). However, interrogation of samples for specific, hot-spot mutations is more practical and cost-effective to perform using PCR-genotyping-based approaches directed to DNA targets of interest, such as KRAS, BRAF, EGFR and others. Information regarding KRAS targets may be found at least in Amicarelli, G., Shehi, E., Makrigiorgos, G. M. and Adlerstein, D. (2007) FLAG assay as a novel method for real-time signal generation during PCR: application to detection and genotyping of KRAS codon 12 mutations. Nucleic Acids Res, 35, e131 and Shi, C., Eshleman, S. H., Jones, D., Fukushima, N., Hua, L., Parker, A. R., Yeo, C. J., Hruban, R. H., Goggins, M. G. and Eshleman, J. R. (2004) LigAmp for sensitive detection of single-nucleotide differences. Nat Methods, 1, 141-147). Information regarding BRAF targets may be found at least in How-Kit, A., Lebbe, C., Bousard, A., Daunay, A., Mazaleyrat, N., Daviaud, C., Mourah, S. and Tost, J. (2014) Ultrasensitive detection and identification of BRAF V600 mutations in fresh frozen, FFPE, and plasma samples of melanoma patients by E-ice-COLD-PCR. Anal Bioanal Chem, 406, 5513-5520. Finally, more information regarding EGFR targets may be found in Kuang, Y., Rogers, A., Yeap, B. Y., Wang, L., Makrigiorgos, M., Vetrand, K., Thiede, S., Distel, R. J. and Janne, P.A. (2009) Noninvasive detection of EGFR T790M in gefitinib or erlotinib resistant non- small cell lung cancer. (lin Cancer Res, 15, 2630-2636, and Guha, M., Castellanos-Rizaldos, E. and Makrigiorgos, G.M. (2013) DISSECT Method Using PNA-LNA Clamp Improves Detection of T790m Mutation. PLOS One, 8, e67782.

Single-step, closed-tube PCR approaches that provide mutation detection directly from human genomic DNA are preferable over multi-step methods, in order to avoid contamination, experimental errors and increased labor. Digital-PCR technology can provide a practical for targeted, quantitative detection of low-level alleles, and may be incorporated into the methods described herein. As used herein, digital amplification (e.g., digital PCR) includes any known technique for digital amplification. Digital amplification is discussed further at least in the following references: Fitarelli-Kiehl, M., Yu, F., Ashtaputre, R., Leong, K. W., Ladas, I., Supplee, J., Paweletz, C., Mitra, D., Schoenfeld, J. D., Parangi, S. et al. (2018) Denaturation-Enhanced Droplet Digital PCR for Liquid Biopsies. (lin Chem, 64, 1762-1771; Vogelstein, B. and Kinzler, K. W. (1999) Digital PCR. Proc Natl Acad Sci USA, 96, 9236-9241; Taly, V. and Huggett, J. (2016) Digital PCR, a technique for the future. Biomol Detect Quantif, 10, 1; and d, M. G. and Huggett, J. F. (2020) The Digital MIQE Guidelines Update: Minimum Information for Publication of Quantitative Digital PCR Experiments for 2020. Clin Chem, 66, 1012-1029. As an alternative, mutation-enrichment methods are often employed to elevate mutation concentrations to levels at which accurate and precise downstream analysis becomes feasible (see at least Milbury, C.A., Li, J. and Makrigiorgos, G.M. (2009) PCR-based methods for the enrichment of minority alleles and mutations. (lin Chem, 55, 632-640). Mutation enrichment methods include enzymatic approaches, that may be applied at the genomic DNA level before PCR (Song, C., Liu, Y., Fontana, R., Makrigiorgos, A., Mamon, H., Kulke, M. H. and Makrigiorgos, G.M. (2016) Elimination of unaltered DNA in mixed clinical samples via nuclease-assisted minor-allele enrichment. Nucleic Acids Res, 44, e146) or during PCR (Fuery, C. J., Impey, H.L., Roberts, N. J., Applegate, T. L., Ward, R. L., Hawkins, N.J., Sheehan, C.A., O'Grady, R. and Todd, A. V. (2000) Detection of rare mutant alleles by restriction endonuclease-mediated selective-PCR: assay design and optimization. (′lin Chem, 46, 620-624). Mutation enrichment approaches also include PCR denaturation-based approaches such as COLD-PCR or wild type ‘blocker’ approaches that enrich mutated alleles during PCR, including LNA/PNA-modified oligonucleotides. More details regarding COLD-PCR may be found at least in Li, J., Wang, L., Mamon, H., Kulke, M. H., Berbeco, R. and Makrigiorgos, G.M. (2008) Replacing PCR with COLD-PCR enriches variant DNA sequences and redefines the sensitivity of genetic testing. Nat Med, 14, 579-584 and Li, J., Wang, L., Janne, P. A. and Makrigiorgos, G.M. (2009) Co-amplification at lower denaturation temperature-PCR increases mutation-detection selectivity of TaqMan- based real-time PCR. (lin Chem, 55, 748-756. More details regarding wild type ‘blocker’ approaches that enrich mutated alleles during PCR and include LNA/PNA-modified oligonucleotides may be found in Wu, L. R., Chen, S. X., Wu, Y., Patel, A. A. and Zhang, D. Y. (2017) Multiplexed enrichment of rare DNA variants via sequence-selective and temperature-robust amplification. Nat Biomed Eng, 1, 714-723; Milbury, C. A., Li, J. and Makrigiorgos, G. M. (2011) Ice-COLD-PCR enables rapid amplification and robust enrichment for low-abundance unknown DNA mutations. Nucleic Acids Res, 39, e2; Murphy, D. M., Bejar, R., Stevenson, K., Neuberg, D., Shi, Y., Cubrich, C., Richardson, K., Eastlake, P., Garcia-Manero, G., Kantarjian, H. et al. (2013) NRAS mutations with low allele burden have independent prognostic significance for patients with lower risk myelodysplastic syndromes. Leukemia, 27, 2077-2081; and Sun, X., Hung, K., Wu, L., Sidransky, D. and Guo, B. (2002) Detection of tumor mutations in the presence of excess amounts of normal DNA. Nat Biotechnol, 20, 186-189.

Once included in the reaction, such blockers operate during every PCR cycle. Depending on input DNA quantity and quality, addition of high blocker concentrations can reduce amplification even for mutated alleles, and this may be particularly problematic when mutation allelic frequencies are low (How-Kit, A., Lebbe, C., Bousard, A., Daunay, A., Mazaleyrat, N., Daviaud, C., Mourah, S. and Tost, J. (2014) Ultrasensitive detection and identification of BRAF V600 mutations in fresh frozen, FFPE, and plasma samples of melanoma patients by E-ice-COLD-PCR. Anal Bioanal Chem, 406, 5513-5520). The requirement to adjust blocker concentration leads to a need for sample-dependent optimization to enable adequate wild type DNA suppression while still allowing enough amplification of rare mutated alleles.

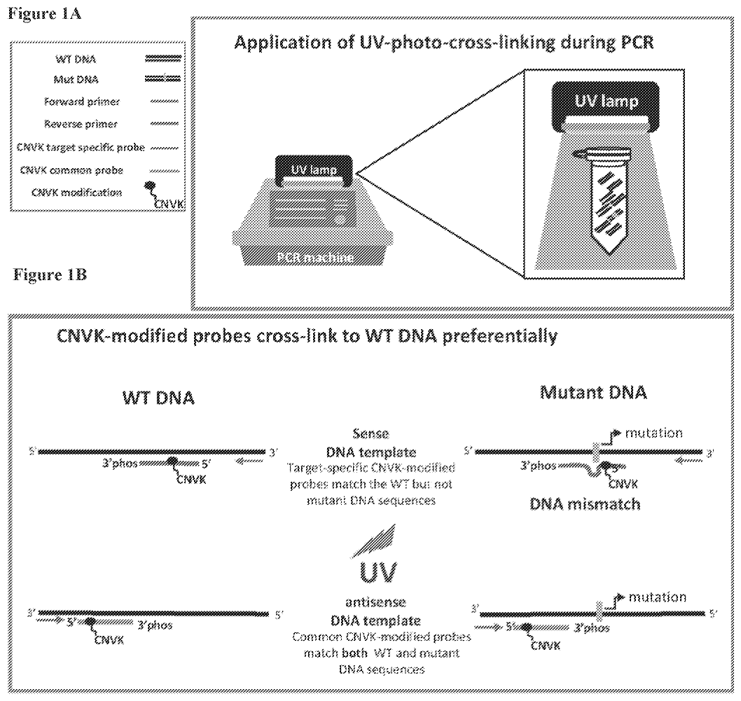

Here, this disclosure introduces a novel enrichment method, UV-mediated Cross-linking Minor-allele Enrichment (UVME), which enables mutation enrichment to start and stop at any cycle during amplification, providing a flexible and sensitive approach. UVME employs UV irradiation for selective photo-cross-linking of wild type DNA to prevent its amplification (FIG. 1A), thereby enabling preferential enrichment of mutated DNA molecules. Oligonucleotide probes matching the sense strand wild type target sequence and incorporating a 3-cyanovinylcarbazole nucleoside (CNVK) modification are employed for cross-linking. CNVK enables rapid UV-enabled cross-linking with thymine or cytosine at −1 position of the opposite DNA strand, and was previously used for site-specific cross-linking with duplex DNA using mutation-specific molecular beacons. UV is applied during the PCR annealing phase, at which the CNVK-modified probes cross-link preferentially to the sense strand of wild type DNA, as compared to mutant DNA and inhibit further amplification (FIG. 1B). Meanwhile, CNVK-modified probes commonly binding a non-target region of the antisense strand of both wild type and mutant DNA are also employed (‘common’ CNVK probes) to inhibit anti-sense strand amplification for both wild type and mutated DNA.

The UVME-PCR program is applied in a single tube, directly from human genomic DNA and includes two stages. First, 10-20 cycles of regular PCR are performed to pre-amplify the target DNA. The UV irradiation is then turned on for 10 seconds at annealing temperature during each subsequent PCR cycle to induce photo cross-linking of the CNVK-modified probes to the target DNA. In view of the probe design, the photo cross-linked duplex of CNVK-modified probes and template DNA suppress the polymerization of both strands of wild type DNA but only one strand of mutant DNA at each cycle, thereby resulting in mutation enrichment during PCR. The mutation-enriched PCR product may be used directly for Sanger sequencing or high resolution melting (HRM) to identify mutations without secondary amplification; alternatively, a PCR-based TaqMan® or ddPCR assay may be performed for endpoint detection (FIG. 1C). UVME mutation enrichment in serial dilutions of mutated genomic DNA and clinical samples containing with low-level mutations were validated.

### Example 3: Exemplary Mutation Enrichment by UVME-PCR Followed by_TaqMan® Real Time PCR Genotyping

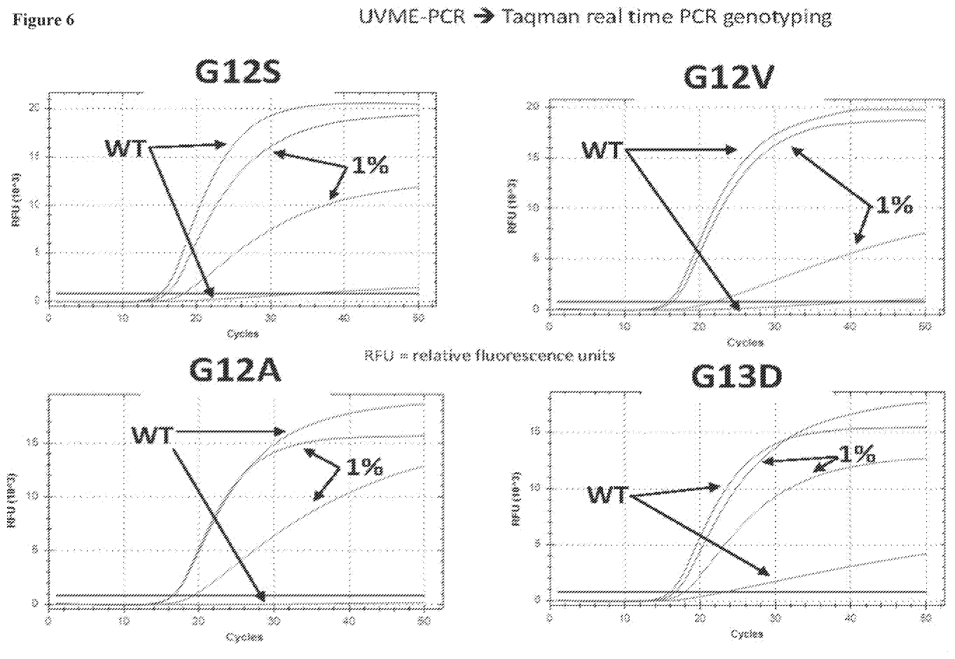

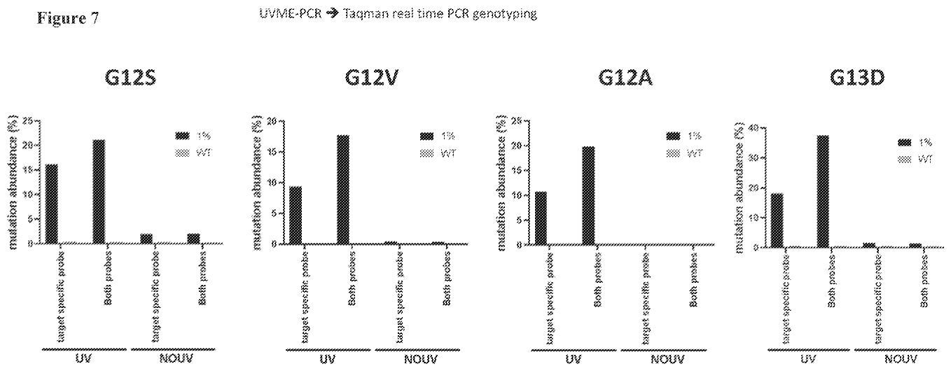

To provide proof of principle for UVME-PCR mutation enrichment, samples with KRAS exon 2 mutations were investigated. A single CNVK-modified probe was employed for enrichment of four common KRAS mutations, G12S, G12V, G12A and G13D that represent the most frequently-encountered KRAS mutations in cancer samples (see Amicarelli, G., Shehi, E., Makrigiorgos, G. M. and Adlerstein, D. (2007) FLAG assay as a novel method for real-time signal generation during PCR: application to detection and genotyping of KRAS codon 12 mutations. Nucleic Acids Res, 35, e131 and How Kit, A., Mazaleyrat, N., Daunay, A., Nielsen, H.M., Terris, B. and Tost, J. (2013) Sensitive detection of KRAS mutations using enhanced-ice-COLD-PCR mutation enrichment and direct sequence identification. Hum Mutat, 34, 1568-1580). Human male genomic DNA (HMC) mixed with 1% DNA from KRAS-mutated cell lines A549-G12S, SW480-G12V, H2009-G12A and LOVO-G13D were initially used in UVME-PCR reactions from genomic DNA. The reaction products were then used for nested PCR employing allele-specific FAM/HEX-labeled TaqMan® probes for mutation identification. Using TaqMan® probes, the PCR threshold difference (delta-Ct, FAM-HEX, and delta-delta CT with and without UV irradiation) may be used to quantify the relative amounts of wild type and mutated DNA and the enrichment in the presence vs. absence of UV irradiation. The data indicate enrichment of all four mutated KRAS alleles in the presence of UV irradiation (FIG. 6). In order to investigate the enrichment when using a single CNVK probe directed to the sense strand, versus two CNVK probes directed to both sense and anti-sense strands (FIG. 1) the experiments were repeated employing either one or two probes per reaction. The data indicate that including only the target specific CNVK-modified probe directed to the sense strand enriches all four KRAS mutations, from 1% to ˜10-20%; however using both the sense probe plus a common CNVK-modified probe directed to the antisense strand increases the mutation abundance even more (from 1% to 15-40%, FIG. 7) indicating a higher mutation enrichment when both probes are used. Accordingly, a combination of target-specific sense and common anti-sense probes were used in this investigation.

### Example 4: UVME-PCR Application on Serial Dilutions of KRAS Mutation-Containing DNA, Followed by Diverse Downstream Assays

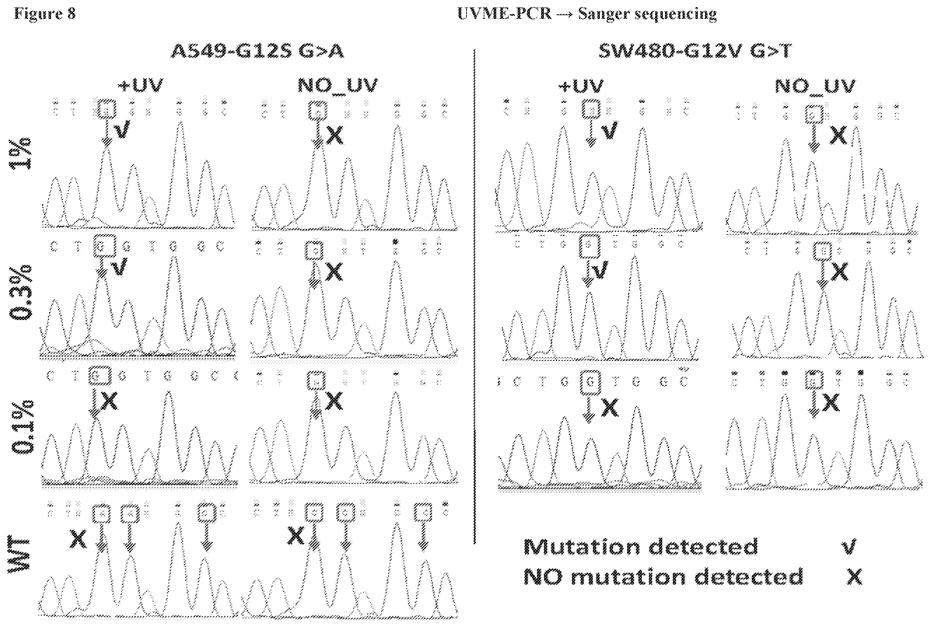

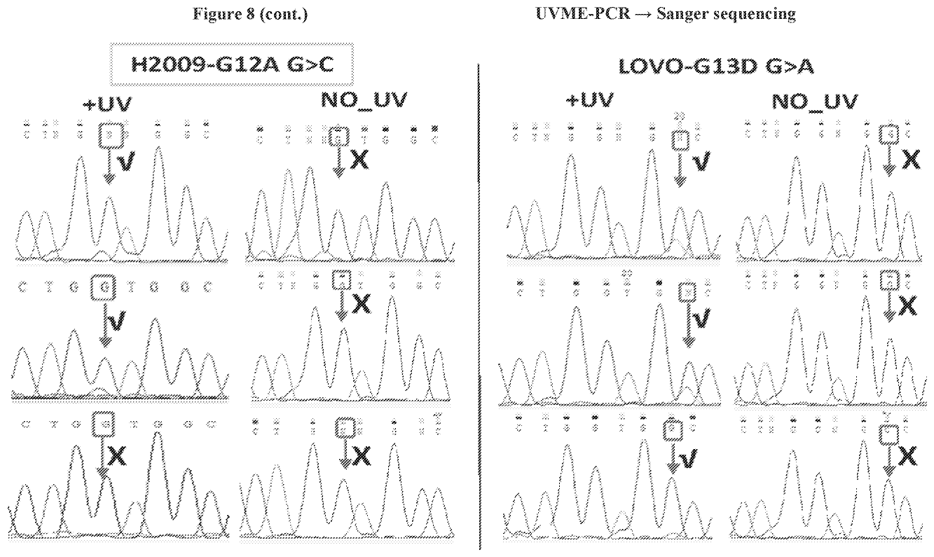

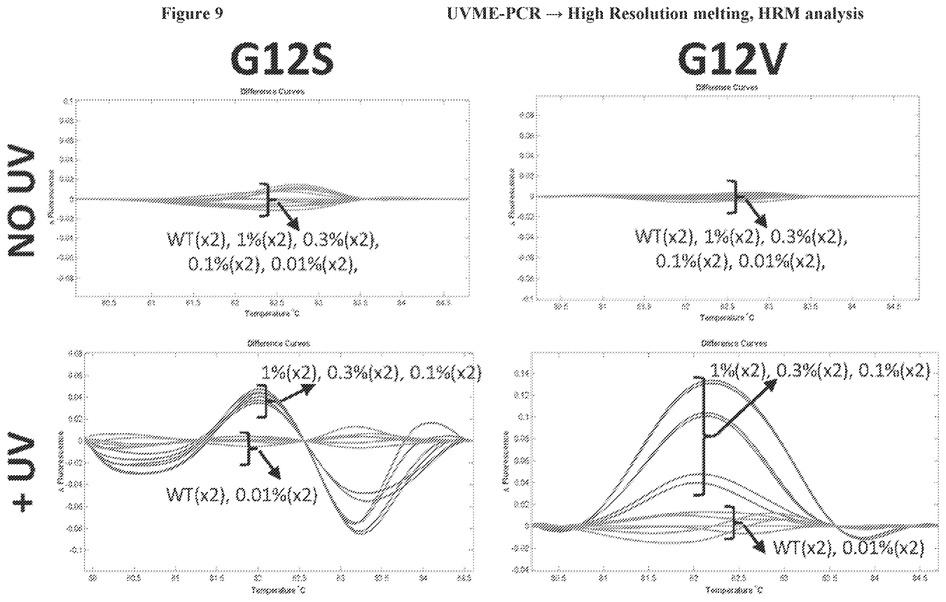

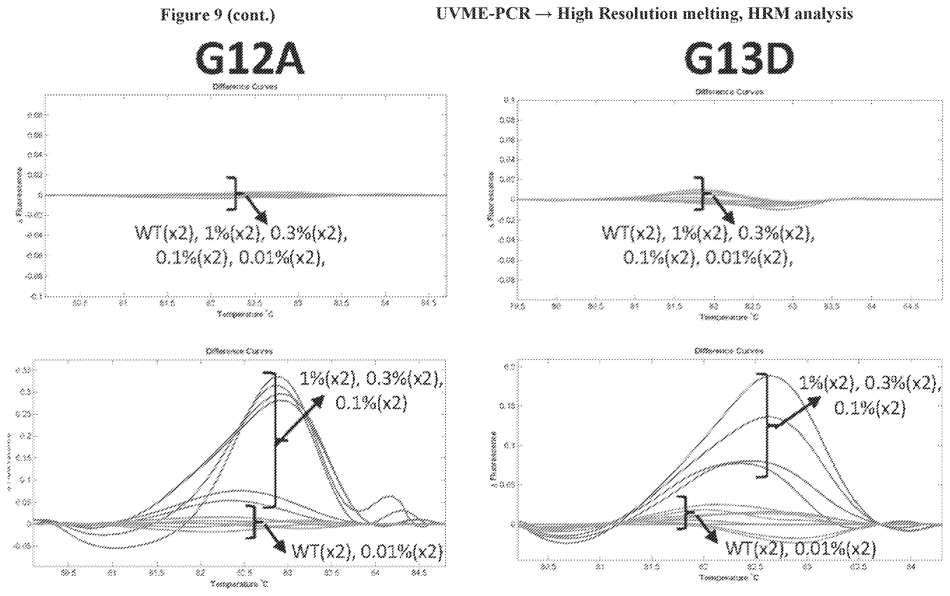

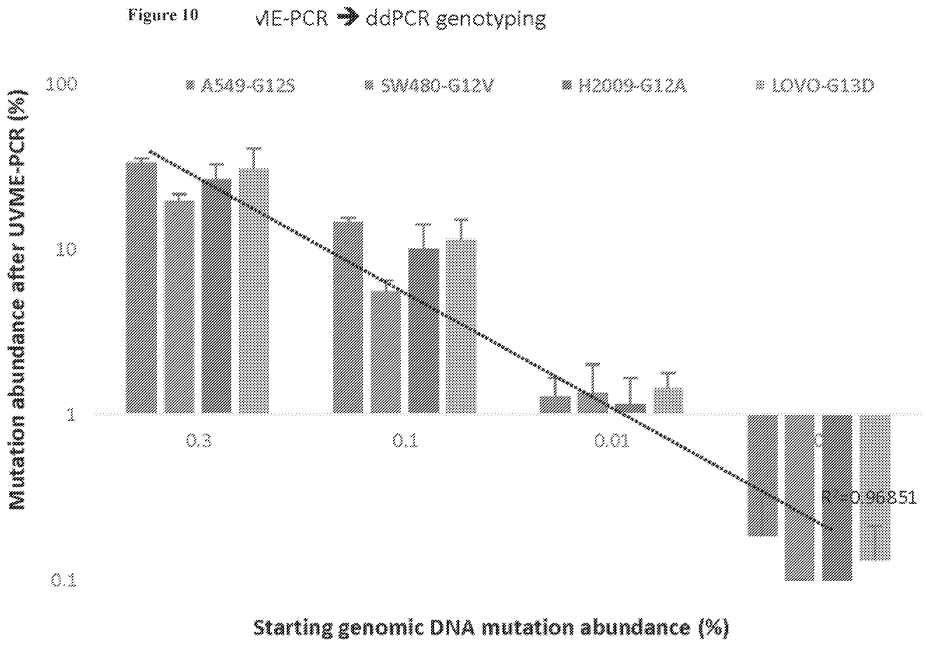

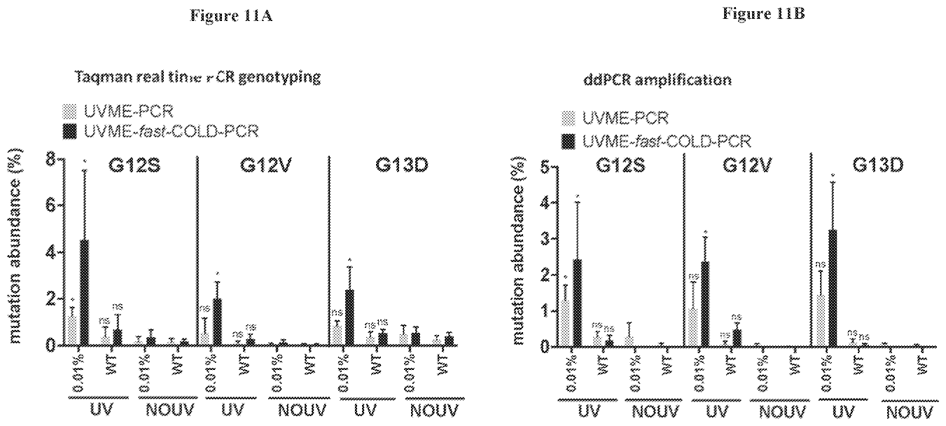

UVME-PCR was next applied on serial dilutions of KRAS mutation-containing DNA to identify limits of detection (LOD), depending on the downstream assay used to identify the mutation. UVME reactions with 30 ng-90 ng input genomic DNA were used for 1%-0.01% allelic frequencies, with the lower dilutions using 90 ng DNA to ensure adequate mutant allele numbers. Sanger sequencing and high-resolution melting (HRM) were then performed directly on UVME-PCR products without secondary amplification to identify mutations. The data indicate detection down to 0.3% MAF via Sanger sequencing (FIG. 8) and 0.1% MAF via HRM (FIG. 9) for all four KRAS mutations in the presence of UV, while no mutations could be detected in the absence of UV for MAF≤1% or wild type DNA. UVME-PCR products were also examined via TaqMan® genotyping assays. The data indicate an LOD of 0.01% (G12S and G13D) and 0.1% (G12V and G12A) after UVME-PCR while in the absence of UV the LOD was 0.3% (G12S, G12V and G13D) and 1% (G12A) (FIG. 2A). To precisely validate the mutation abundance, ddPCR was also employed on UVME-PCR products. Data indicate an LOD down to MAF ˜ 0.01% and up to 100-fold enrichment for four KRAS mutations (FIG. 2B). In the absence of UV, the ddPCR LOD was ˜ 0.1%, essentially dictated by the number of droplets per run in the standard Biorad ddPCR format. Additionally, when plotted for the lower level mutations (<1%) the starting mutation abundance indicate a correlation (R2˜0.97) to the final mutation abundance, and this was uniform for all four KRAS mutations studied (FIG. 10). Therefore, following UVME, the ddPCR result may be used for approximate quantification of the input mutation fraction. In summary, the KRAS mutation dilution experiments indicate that UVME-PCR mutation enrichment using the FIG. 1 format leads to KRAS mutation enrichments of ˜100-fold and an LOD of 0.01-0.3% depending on downstream assay used.

### Example 5: Combination of Fast-COLD-PCR with UVME-PCR (UVME-Fast-COLD-PCR) to Boost Enrichment for the Tm-Decreasing KRAS Mutations G12S, G12V and G13D

The mutation enrichment by UVME-PCR relies on selective UV-cross-linking at the annealing step while the enrichment by COLD-PCR is based on selective melting during denaturation (Li, J., Wang, L., Mamon, H., Kulke, M. H., Berbeco, R. and Makrigiorgos, G. M. (2008) Replacing PCR with COLD-PCR enriches variant DNA sequences and redefines the sensitivity of genetic testing.Nat Med, 14, 579-584; Castellanos-Rizaldos, E., Liu, P., Milbury, C. A., Guha, M., Brisci, A., Cremonesi, L., Ferrari, M., Mamon, H. and Makrigiorgos, G.M. (2012) Temperature-Tolerant COLD-PCR Reduces Temperature Stringency and Enables Robust Mutation Enrichment. (lin Chem, 58, 1130-1138; and Milbury, C. A., Li, J. and Makrigiorgos, G. M. (2009) COLD-PCR-enhanced high-resolution melting enables rapid and selective identification of low-level unknown mutations. (lin Chem, 55, 2130-2143). Thus, combination of these two enrichment methods was explored to further boost the mutation enrichment achieved during a single PCR reaction. As a proof of principle, 90 μg input HMC DNA containing 0.01% of the Tm-decreasing mutations G12S (G>A), G12V (G>T) and G13D (G>A) were employed in the combination of temperature-tolerant (TT) fast-COLD-PCR and UVME-PCR (UVME-TT-fast-COLD-PCR). Instead of a regular denaturation temperature at 95° C., critical denaturation temperatures at 81.2° C. (9 cycles, step 1) and 82.2° C. (11 cycles, step 2) were used to selectively amplify the mutated DNA copies during the pre-amplification stage of UVME-PCR. The products were then screened via TaqMan® genotyping (FIG. 11A) and ddPCR (FIG. 11B). The data indicate an increase in mutation enrichment via UVME-TT-fast-COLD-PCR, as compared to UVME-PCR for both TaqMan® and ddPCR approaches. On the other hand, wild type samples run in parallel demonstrated a somewhat higher background mutation rate when UVME-TT-fast-COLD-PCR was applied.

Accordingly, while the mutation enrichment increased, the overall limit of detection showed no clear improvement following serial addition of COLD-PCR.

### Example 6: UVME-PCR Application to a p53 Hotspot Mutation (p53-C275G)

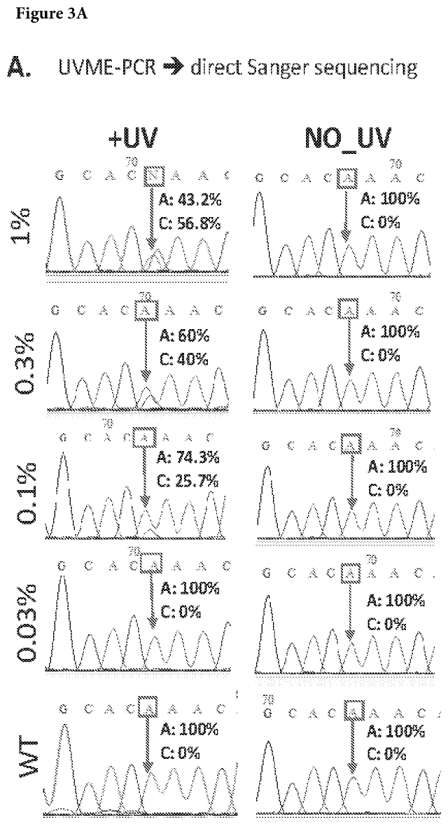

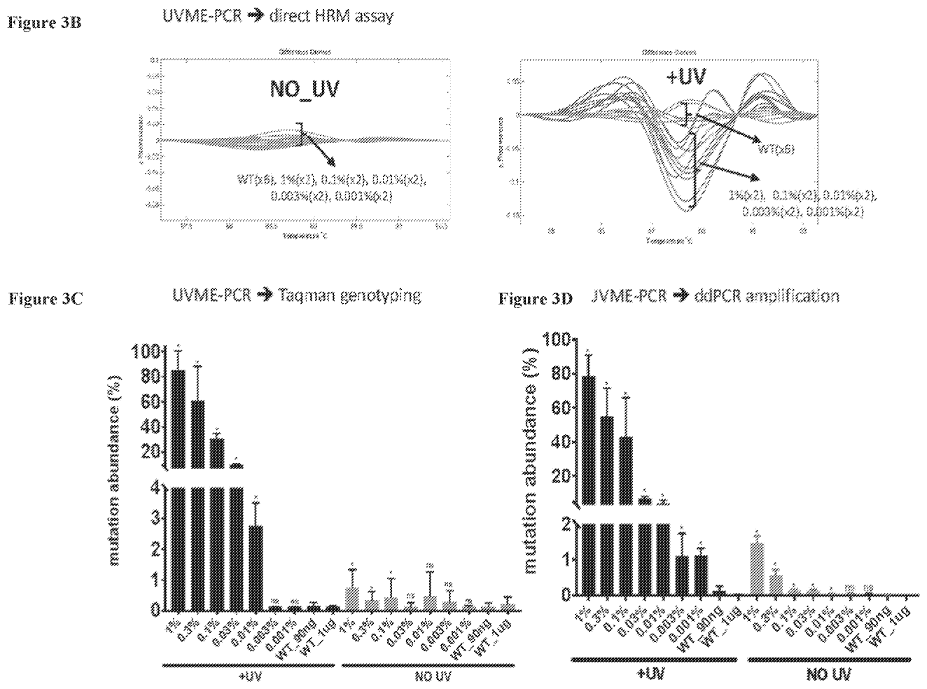

To apply UVME-PCR to a second DNA target, other than KRAS, the p53 mutation C275G which is frequently encountered in lung cancer was investigated (Li, J., Milbury, C.A., Li, C. and Makrigiorgos, G.M. (2009) Two-round coamplification at lower denaturation temperature-PCR (COLD-PCR)-based sanger sequencing identifies a novel spectrum of low-level mutations in lung adenocarcinoma. Hum Mutat, 30, 1583-1590; and Olivier, M., Eeles, R., Hollstein, M., Khan, M.A., Harris, C. C. and Hainaut, P. (2002) The IARC TP53 database: new online mutation analysis and recommendations to users. Hum Mutat, 19, 607-614). Genomic DNA from PFSK cell line containing a homozygous p53-C275G mutation was serially diluted into wild type DNA, 1%-0.001%, using a total of lug DNA per PCR reaction. This corresponds to about 330,000 genomic copies which should contain 3-4 mutant molecules in the reaction at the 0.001% level. Varying amounts of input DNA up to lug per reaction were used as input in UVME-PCR reactions. The reaction products were then directly screened via Sanger sequencing, HRM, TaqMan® genotyping and ddPCR. Sanger sequencing indicated a limit of 0.1% MAF in the presence of UV irradiation, while no mutation could be detected in the absence of UV for the dilutions tested (FIG. 3A). HRM analysis indicated presence of mutations down to 0.001% in the presence of UV while no difference was evident in samples without UV (FIG. 3B). TaqMan® genotyping indicated detection down to 0.01% indicating a ˜300-fold mutation enrichment (FIG. 3C) and ddPCR indicated detection down to MAF ˜0.001%, corresponding to 1000-fold mutation enrichment (FIG. 3D). In summary, using high input genomic DNA (1 μg) per reaction, UVME-PCR enrichment plus HRM/ddPCR for the hotspot p53 mutation C275G enables detection down to 1 in 105 alleles.

### Example 7: Application of UVME-PCR for Detection of KRAS Mutations in Clinical Tumor Samples

For a proof-of-principle application of UVME-PCR in clinical samples, DNA from ten lung tumors were used. Six of these samples (TL3, TL30, TL5, TL6, TL15 and TL8) had been previously shown to harbor low-level KRAS mutations using a sensitive PNA-PCR based approach while four samples (TL80, TL13, TL81 and NL77) along with HMC DNA were wild type. More details regarding this PNA-PCR based approach may be found at least in Fujimoto, K., Yamada, A., Yoshimura, Y., Tsukaguchi, T. and Sakamoto, T. (2013) Details of the Ultrafast DNA Photo-Cross-Linking Reaction of 3-Cyanovinylcarbazole Nucleoside: Cis-TransIsomeric Effect and the Application for SNP-Based Genotyping. Journal of the American Chemical Society, 135 and Liu, Q., Guo, X., Xun, G., Li, Z., Chong, Y., Yang, L., Wang, H., Zhang, F., Luo, S., Deng, Z. et al. (2020) Argonaute-mediated system for supersensitive and multiplexed detection of rare mutations. bioRxiv, 803841). These samples were re-examined from genomic DNA using regular ddPCR and mutations at ˜1-45% mutation abundance was ascertained for the six samples previously found to be positive (FIG. 4A). When UVME-PCR was applied prior to ddPCR the mutation abundance detected via ddPCR was increased to ˜30-90% (FIG. 4A). No mutations for the remaining four samples or HMC DNA were detected. Samples were then also examined via Sanger sequencing as the endpoint detection method. The mutations were detected in the six positive samples via UVME-Sanger (FIG. 4B). No mutations could be detected via Sanger sequencing in the absence of UV cross-linking (NO UV) in five out of six positive samples. The data indicate that UVME mutation enrichment enabled Sanger sequencing-based detection of mutations in low-purity clinical samples and magnified ddPCR mutation signals as compared to regular ddPCR.

Based at least on the working examples and description provided herein, light-based, site-specific nucleic acid cross-linking methodologies have been disclosed that are useful for enrichment of mutations, such as directly from genomic DNA and/or in a closed tube format. For example, CNVK incorporated into DNA-hybridized oligonucleotides cross-links with pyrimidines on the opposite DNA strand in a rapid reaction with ˜365 nm UVA radiation which otherwise produces no detectable DNA damage (see Yoshimura, Y. and Fujimoto, K. (2008) Ultrafast reversible photo-cross-linking reaction: toward in situ DNA manipulation. Org Lett, 10, 3227-3230) and was previously shown to induce site-specific crosslinking with duplex DNA using mutation specific molecular beacons (see Fujimoto, K., Yamada, A., Yoshimura, Y., Tsukaguchi, T. and Sakamoto, T. (2013) Details of the Ultrafast DNA Photo-Cross-Linking Reaction of 3-Cyanovinylcarbazole Nucleoside: Cis-Trans Isomeric Effect and the Application for SNP-Based Genotyping. Journal of the American Chemical Society, 135.). The ability to apply UV at any point during PCR cycling provides the flexibility to apply target pre-amplification without UV using regular PCR in the first few cycles, to boost mutated DNA copies then switch to WT DNA-specific UV-crosslinking to amplify selectively mutation-containing DNA alleles. Unlike mutation enrichment approaches that require a separate pre-amplification step prior to enrichment (see Liu, Q., Guo, X., Xun, G., Li, Z., Chong, Y., Yang, L., Wang, H., Zhang, F., Luo, S., Deng, Z. et al. (2020) Argonaute-mediated system for supersensitive and multiplexed detection of rare mutations, bioRxiv, 803841, and Song, J., Hegge, J. W., Mauk, M. G., Chen, J., Till, J. E., Bhagwat, N., Azink, L.T., Peng, J., Sen, M., Mays, J. et al. (2019) Highly specific enrichment of rare nucleic acid fractions using Thermus thermophilus argonaute with applications in cancer diagnostics. Nucleic Acids Research, 48, e19-e19 for examples of such approaches) the ability to combine pre-amplification plus enrichment steps, in a single tube directly from genomic DNA has practical advantages such as convenience, speed and reduced contamination risk. As with all approaches that employ pre-amplification, there is a risk of introducing PCR errors which may then be enriched during the UV-crosslinking stage and result in false-positives. An indication for increased PCR noise was evident when COLD-PCR was applied for 20 cycles pre-amplification (FIG. 10). Accordingly, pre-amplification cycles should be limited to reduce the possibility for PCR errors. However, using pre-amplification up to 10 cycles did not yield any evidence of increased mutational background.

The methods provided herein may use a single CNVK-modified probe to enrich multiple KRAS mutations on a single target. Multiplexing UVME reactions to enrich multiple targets simultaneously by designing CNVK-modified probes against several targets of interest are also contemplated.

In the absence of a PCR machine that incorporates a UV lamp, open-lid PCR may be used and light, such as UV, may be applied to PCR plates using an external UV lamp. Open lid PCR generally requires higher number of cycles relative to closed, heated-lid PCR to achieve a given amplification. Incorporation of the UV source inside the PCR machine and automated control are anticipated embodiments of UVME application. Implementation or UVME-PCR in a real-time PCR fashion would provide additional advantages, such as switching the UV on/off automatically when the reaction reaches certain PCR thresholds using real time PCR based either on DNA-binding dyes or on oligonucleotide probes (see Tyagi, S. and Kramer, F.R. (1996) Molecular beacons: probes that fluoresce upon hybridization. Nat Biotechnol, 14, 303-308, as an example of probes that may be utilized).

### Example 8: An Embodiment Comprising a Cycling Reaction for Selective Cross-Linking without Amplification

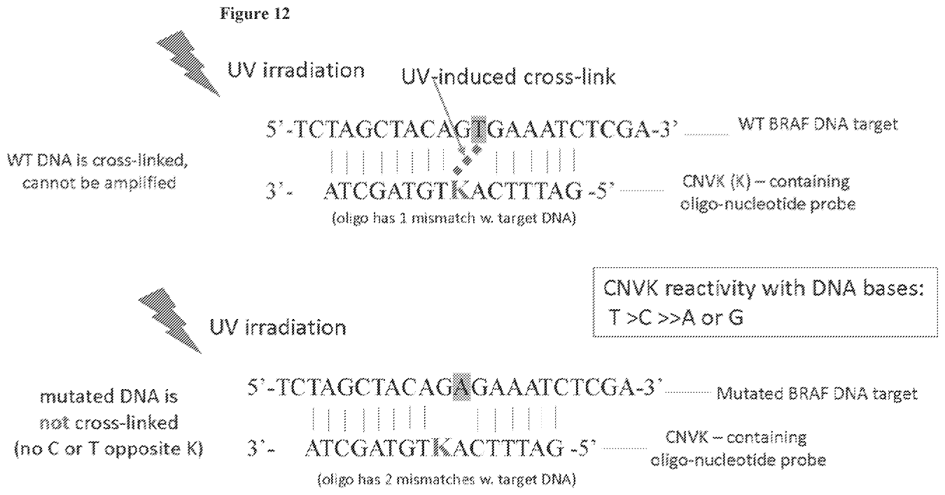

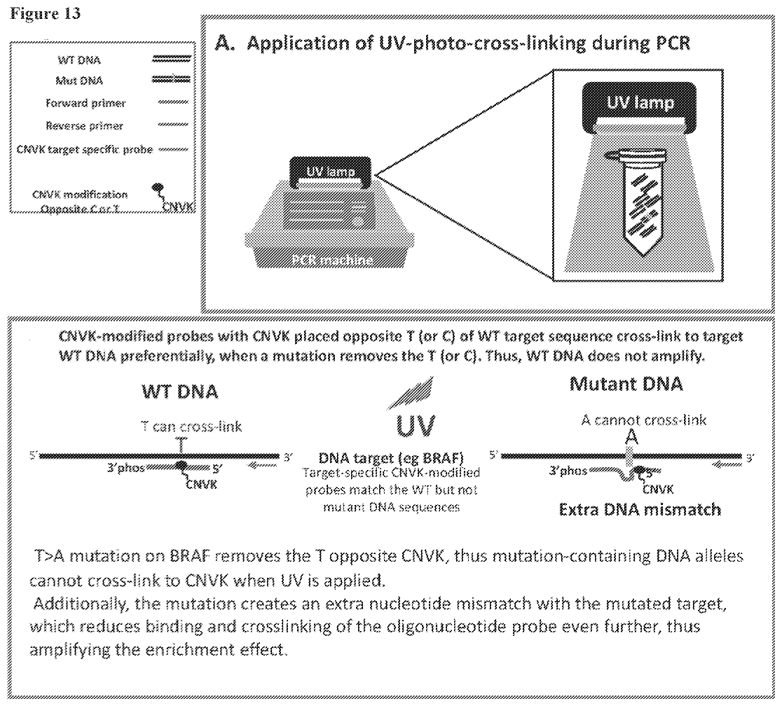

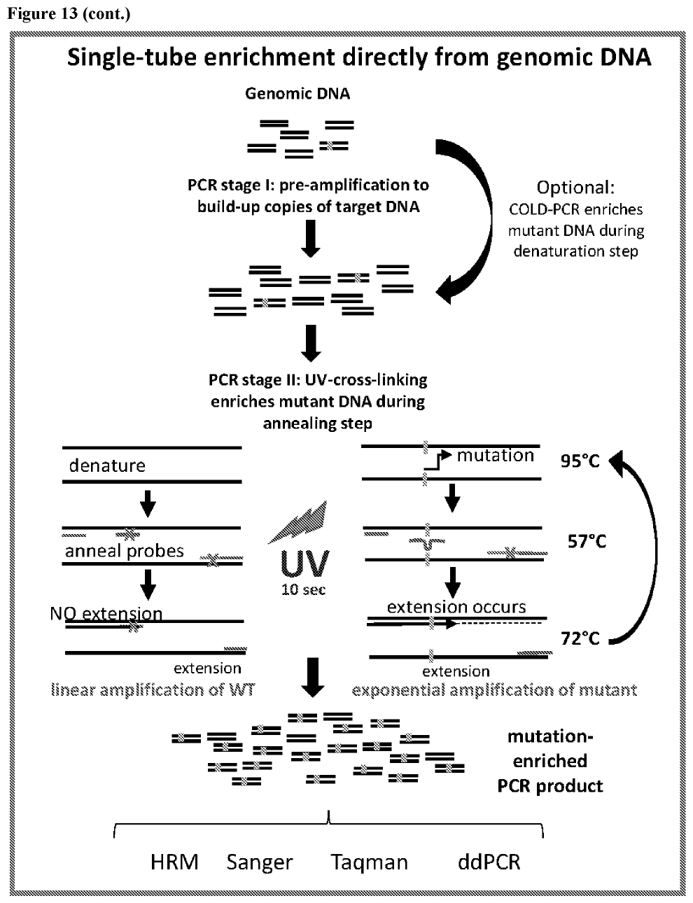

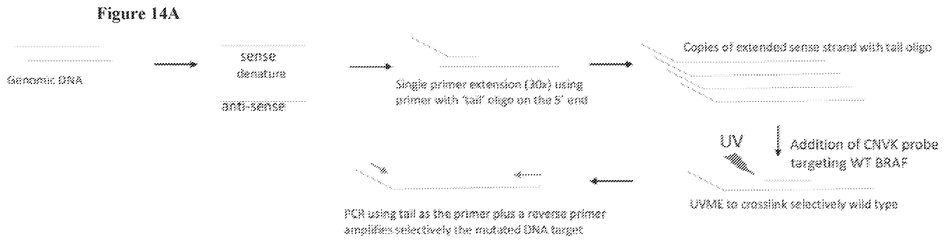

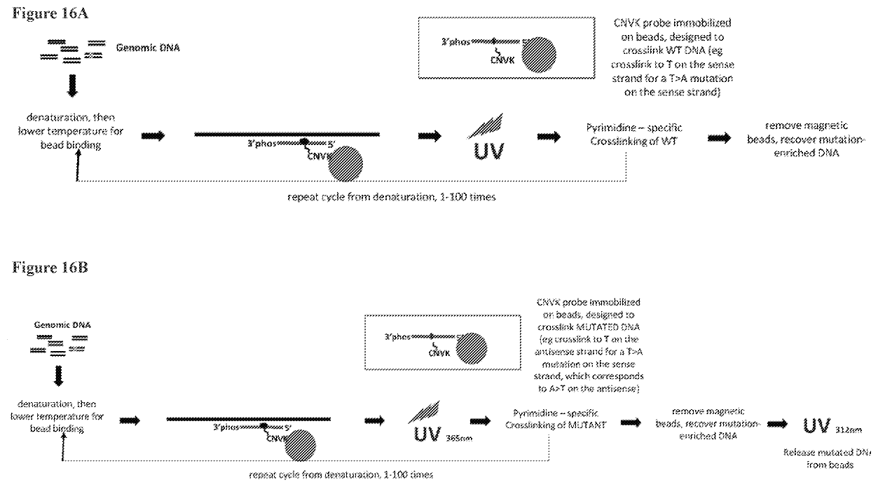

Additionally, the methods described herein may be applied by using a scheme described in FIG. 12 and/or FIG. 13 in a cycling-fashion and in the absence of any PCR components (i.e., without polymerase or PCR primers). In this embodiment, only the nucleic acid target and non-target sequences, as well as the oligonucleotide comprising a photo-activatable molecule are present in the solution. In some embodiments, selective cross-linking of the non-target (e.g., wild type) nucleic acid may be applied either once, or in repeated fashion which includes a denaturation of double stranded nucleic acids, then reducing the temperature to allow the oligonucleotides to anneal to the nucleic acid molecules, then applying light (e.g., UV or visible light) to selectively cross-link the nucleic acid molecules; and repeating the cycle multiple times (e.g., 2-100) times as necessary to remove almost completely the non-target nucleic acid sequences while sparing the target nucleic acids. An example for the T>A BRAF mutation enrichment in the absence of PCR components (no polymerase, no dNTPs, no primers) is demonstrated in FIG. 16. In this example, only a sample with genomic DNA containing a 5% BRAF mutation and the CNVK probe shown on FIG. 12 were used. Various CNVK probe concentrations were tested. Cycling between 95° C. (denaturation) and 45° C. (hybridization plus UV) was performed for 10 cycles. Following this, the product was examined for BRAF mutations using digital PCR. Enrichment for the mutated alleles was observed following UV treatment, for all CNVK concentrations used. No enrichment is present in the absence of UV.

The mechanism via which preferential crosslinking occurs is the same as the one described during a PCR reaction. The oligonucleotide will hybridize with target, as well as non-target sequence, each time the temperature is reduced to 45° C. Upon photo-activation, the oligonucleotide binds selectively to a fraction of the non-target (e.g., wild type) sequences, while it does not crosslink to target sequences that may be present. By repeating many cycles, bigger and bigger fractions of non-target sequences become cross-linked. There is no amplification involved at any step, but the reaction progressively results in fewer and fewer non-target sequences being able to amplify during a subsequent amplification reaction. Following this photoactivation step(s), standard amplification reagents are added to the sample, followed by amplification of the target sequences that have not been cross-linked. Thus, in this embodiment, selective cross-linking is applied independent of simultaneous amplification (e.g, the amplification is applied after the fact). Note that the amplification can be one of a variety of methods, such as PCR, isothermal, etc., and can take place either as solution-based amplification, solid-support-based amplification, and the like, such as amplification on beads or on glass slides as used during next-generation sequencing.

Generally, there may be amplification to translate selective cross-linking to a desired target-enrichment advantage, but photo-crosslinking itself can either take place during amplification, or before amplification (at the genomic DNA level) followed by amplification after the cross-linking protocol is completed. The latter approach has major advantages, as PCR errors are avoided and the methods remain open to any possible form of amplification, not just PCR. It is also practically very easy to accomplish in practice.

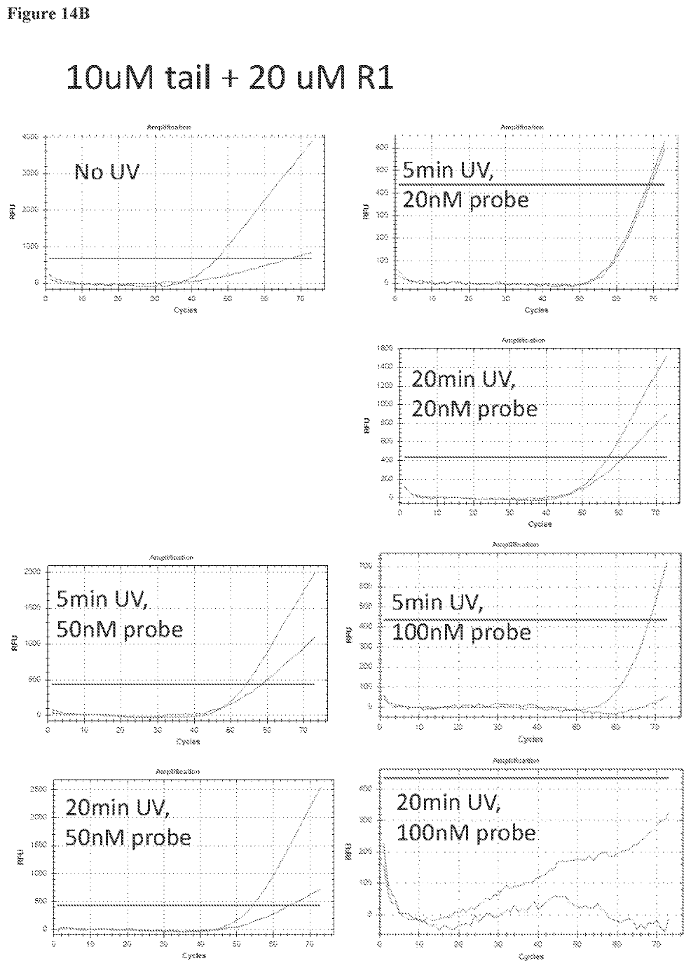

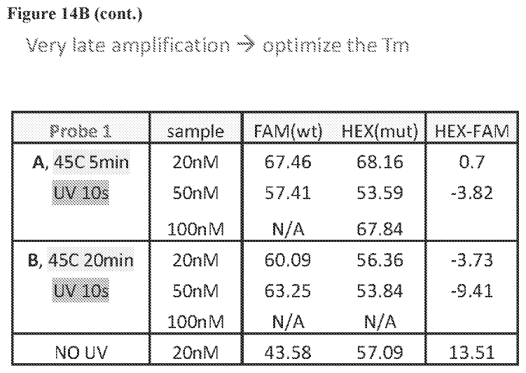

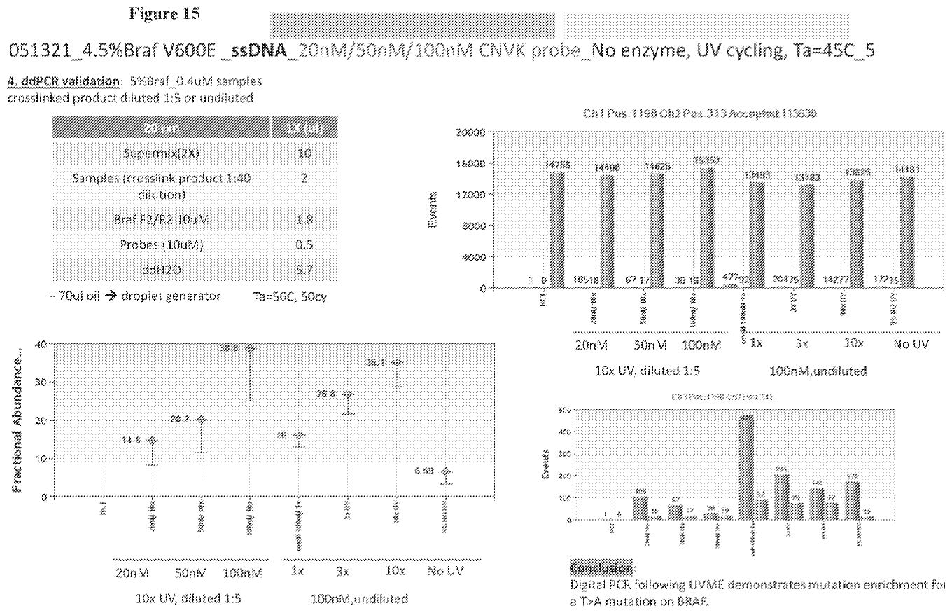

FIGS. 14A and 14B describe a representative workflow followed to amplify only the sense DNA strand (i.e., the strand that comprises the target and non-target nucleic acid sequence) that is selectively cross-linked and to ignore the anti-sense DNA strand which is not contacted by the oligonucleotide comprising a photo-activatable molecule. Specifically, prior to cross-linking, a single primer extension is applied on the anti-sense strand to create copies of the sense strand containing an oligonucleotide ‘tail’ on the 5′end. Following purification to remove the tail-containing primer, UVME is applied to selectively cross-link the wild type-sense strand while leaving mutated-sense strand intact. Subsequently, PCR using the tail as one primer plus a reverse primer amplifies only the sense strand that was selectively contacted by the oligonucleotide.

There are additional ways to focus PCR amplification only on the selectively-cross-linked DNA strand and ignore the opposite strand. These approaches are known to those skilled in the art and may be adapted to work with UVME. For example, starting with genomic DNA one may start with application of UVME and then apply a TdT reaction (terminal deoxynucleotidyl-transferase) to add poly-adenine tails to the 3′ends of all DNA molecules. Following addition of the poly A tails on the 3′end, one may apply PCR using a poly-dT reverse primer that binds to poly-A in conjunction with a forward primer that corresponds to the sense DNA strand that was selectively cross-linked. Alternatively, starting with genomic DNA, one may start with application of UVME and then apply asymmetric PCR that includes un-equal amounts of the forward vs. reverse primer to amplify preferentially either the sense or anti-sense DNA strands, as needed.

### Example 9: An Embodiment Encompassing Immobilization of Oligonucleotides Comprising a Photo-Activatable Molecule on Solid Support to Remove Wild Type DNA

Oligonucleotides comprising a photo-activatable molecule are immobilized onto a solid support (e.g., solid supports such as magnetic beads, or any solid support disclosed herein or known in the art). Preferential cross-linking of targeted DNA may then take place, either in a single cross-linking step or in a cycling step. FIG. 16A provides a representative, non-limiting example. Biotinylated CNVK-containing oligonucleotides are bound to magnetic streptavidin beads. DNA is then added to the solution containing the beads. The following may then be repeated: denature DNA, lower the temperature to allow the DNA to hybridize to CNVK probes bound to the beads, and then apply UV light to induce cross-links with the wild type DNA. This cycle may optionally be repeated 2-100 times. After cross-linking most or all the targeted DNA on the beads, the beads are removed from the solution via magnetization and the mutated DNA remains in solution and may be used for mutation analysis. One may also optionally repeat the bead-binding step (e.g., for a second time, third time, etc.) to achieve even higher enrichment prior to mutation analysis.

Alternatively, oligonucleotides comprising photo-activatable molecules may be designed to hybridize to target nucleic acids sequences (rather than non-target sequences as shown in FIG. 16A). For example, CNVK-modified probes may be immobilized on a solid support to remove mutated DNA. Probes are then exposed to light adapted to a wavelength to reverse the cross-linked DNA. UV irradiation at 365 nm cross-links CNVK to pyrimidines C/T, while irradiation with UV at 312 nm reverses the cross-link. Therefore, mutated DNA is selectively cross-linked to the beads by targeting the cross-linking of the mutated DNA. For example, see FIG. 16B. CNVK probes are designed to hybridize to the opposite DNA strand, thereby cross-linking the DNA strand where an A>T mutation is present (instead of the previously shown T>A on the sense strand). The mutation-containing DNA is then cross-linked to CNVK probes immobilized on beads, while the wild type DNA will not cross-link. Once the mutated DNA is cross-linked to the beads, the beads are removed via magnetization, and are introduced to a clean buffer-only solution. Then UV irradiation is applied at 312 nm UV wavelength, as opposed to the 365 nm used for cross-linking. This UV irradiation reverses the cross-linking and releases the mutated DNA from the beads into the solution, thereby allowing amplification and analysis of the enriched mutated DNA.

### Example 10: An Embodiment Encompassing Use of Modified DNA Bases to Modify Cross-linking rates

In some embodiments, the methods disclosed herein may include incorporating a modified base. As a non-limiting example, because CNVK reacts strongly with T, but also with C, but not with G or A, the present pyrimidine-dependent mutation enrichment via CNVK-containing oligonucleotide probes may be applied to enrich most single nucleotide changes by cross-linking either to the sense or to the antisense strand as the application dictates. For example, for sense strand mutations T>A, T>G and T>C that remove a pyrimidine (OR exchange a T with a C which has lower reactivity with CNVK relative to T) the concept of FIG. 12 applies as shown. For mutations A>T, A>C or A>G in the sense strand, an oligonucleotide is designed to target the corresponding base-changes in the anti-sense strand, i.e., T>A, T>G, T>C and apply the same concept shown above to cross-link the antisense DNA target strand. For mutations G>A, G>T and G>C in the sense strand, an oligonucleotide is designed to target the corresponding base-changes in the anti-sense strand, i.e. C>T, C>A and C>G and apply the same concept. Finally, for sense strand mutations C>A and C>G the concept of FIG. 12 applies as shown. The only situation that selective cross-linking of wild type DNA cannot be accomplished is C>T mutations, since neither the sense strand mutation (C>T) nor the corresponding bottom strand mutation (G>A) would be cross-linked to a higher extend with wild type DNA as opposed to a mutated target (in this case, one may use modified bases. Finally, for mutations creating micro-deletions or insertions, a similar strategy may be adopted as in FIG. 12, to cross-link specifically the wild type target by placing CNVK opposite a C or T that is removed following the deletion or insertion.

In order to enhance enrichment in the presence of C>T mutations, modified DNA bases may be incorporated into the nucleic acid molecules that change the DNA bases to manipulate bases available for CNVK cross-linking. In one embodiment, modified bases are incorporated into nucleic acid molecules by single primer extension, or PCR by using methylated dCTP (5mC) in the place of standard dCTP. Following this step, a potential C>T mutation would be converted to a 5mC>T mutation. CNVK shows a preference for 5mC over T. Accordingly, C may also be replaced with 5mC prior to applying the enrichment methods described herein. Similarly, dUTP may be used in PCR extension in the place of dTTP in this same method. Additionally, dITP (I=Inosine) may be used in the place of dCTP. Any other base analogue that changes the relative reactivity between CNVK and a DNA base, to achieve the same endpoint, i.e., enabling lower or higher reactivity of CNVK with the nucleic acid sequences. Additional examples of modified deoxynucleotide-triphospates (modified-dNTPs) include tri-phosphates with 6N—methyladenine, 8-oxo-guanine, 4N-methylcytosine, 5-substituted pyrimidine and 7-substituted 7-deazapurine dNTP, or all those modified dNTPs included in commercial catalogues (e.g., without limitation include the following lists of commercially available dNTPs on the World Wide Web, such as jenabioscience.com/nucleotides-nucleosides/nucleotides-by-structure/nucleotide-trove/modified-dntps or trilinkbiotech.com/products-services/nucleoside-triphosphates-nucleotides/base-modified-nucleoside-triphosphates.html?product_list). Any other dNTP that has been described in the literature or is available in commercial catalogues may be used.

For example, in order to enrich mutations that convert cytosine to any of the other three bases, (i.e., C>T, C>A or C>G) a few cycles of PCR using methylated deoxy-cytosine-triphosphate (d5mCTP) in the place of regular dCTP are conducted to convert all C to 5mC in the nucleic acid molecules. Then, CNVK-containing probes that are complementary to the wild type are hybridized to the target sequences, with CNVK placed at the −1 position opposite the targeted 5mC. If a mutation is present, the CNVK probes will cross-link strongly with the wild type form that contains 5mC but very weakly with mutated targets containing A, G, or T in the place of 5mC since there is less reactivity as compared to 5mC and the mutated nucleotide. All mutations from C to any other base will lead to enrichment of the target nucleic acid sequences.

Unexpectedly, it was determined that by placing a photo-activatable molecule at a particular position (e.g., CNVK at position −1 relative to the mutation position) as opposed to anywhere else on the oligonucleotide sequence), formation of a mismatch directly opposite the CNVK is enabled, which reduces reactivity with the target allele sequence even more than expected by simply accounting for the temperature-based reduction in hybridization.

### Example 11: An Embodiment Encompassing Application of UVME Selective Cross-Linking of Wild Type Samples Based Only on the Presence of Mismatches Between Mutated DNA and CNVK-oligonucleotide probes (‘mismatch-dependent UVME’)

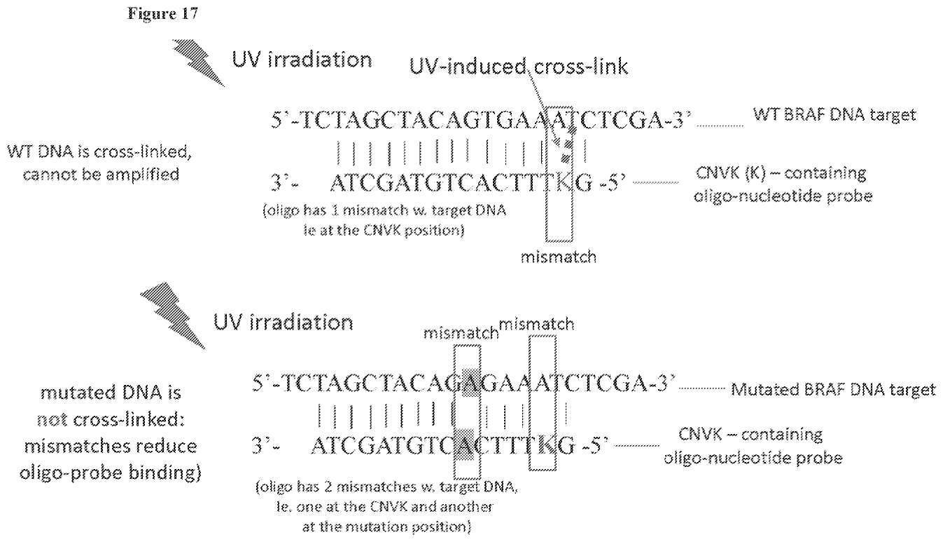

In some embodiments, a photo-activatable molecule (e.g., CNVK) may be placed a few bases away from the mutation, opposite any desired base, such as C or T of the wild type sequence, and not necessarily opposite the C or T that comprises the hotspot mutation of interest (FIG. 17). When the oligonucleotide probe hybridizes to the nucleic acid sequence, presence of a mutation creates a mismatch which prevents effective hybridization of the probe to the target nucleic acid sequence, while full hybridization to the wild type (e.g., the non-target sequence) remains un-hindered as the probe sequence matches the wild type sequence. In this manner, the PCR approach shown on FIG. 13 may be applied in a similar manner and mutated DNA alleles are preferentially enriched over wild type alleles. Thus, the wild type targets amplify only linearly, while mutated targets amplify exponentially.

In addition, a second oligonucleotide probe may be designed to target the opposite DNA strand in the same manner (FIG. 1B). In this case both DNA strands of the wild type become un-amplifiable, while one strand of the mutated alleles can still be amplified, resulting in mutation enrichment during PCR (FIG. 1C).

Examples, detailed methods, and results from the application of the mismatch-dependent embodiment of UVME where both strands are cross-linked are shown on FIGS. 2-5 and 7 for KRAS and p53 targets.

The mismatch-dependent UVME approach described in this representative working example may be applied at every step during a PCR reaction.

### Example 12: An Embodiment Encompassing Application of UVME in Multiplexed Format by Using Split-Probes of Generic Design

In order to enrich for multiple nucleic acid target sequences simultaneously, can employ multiple oligonucleotide probes comprising photo-activatable molecules for multiple possible mutations. Then a pyrimidine-dependent (e.g., Example 2) or a mismatch-dependent approach (e.g., Example 11) may be applied simultaneously to enrich nucleic acids comprising mutations at many positions. The mutation-enriched DNA may then be sent for sequencing (e.g., using any sequencing or detection method described herein) to identify presence or absence of mutations on multiple targets.

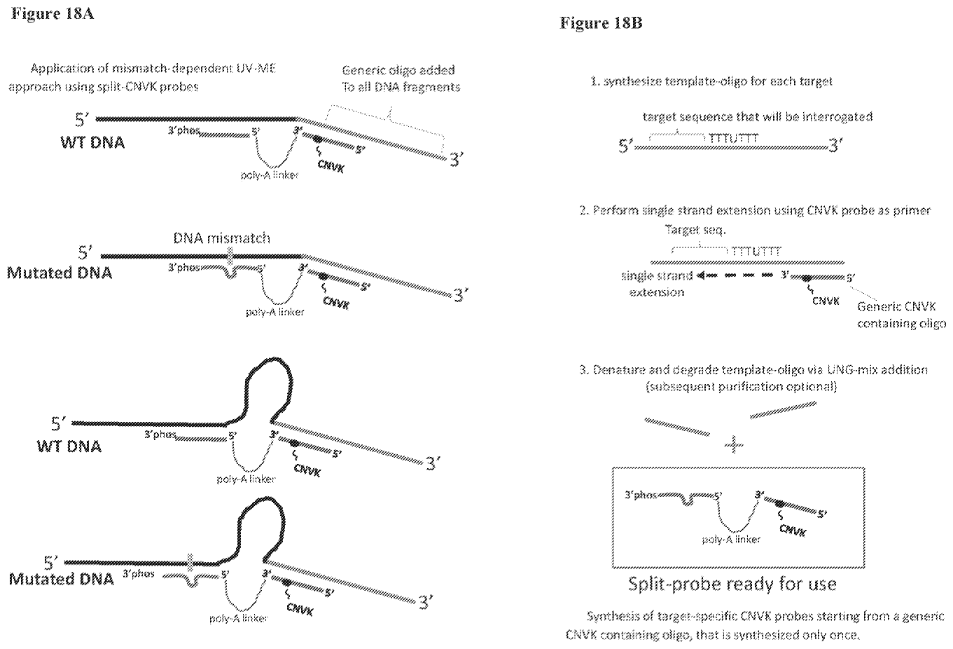

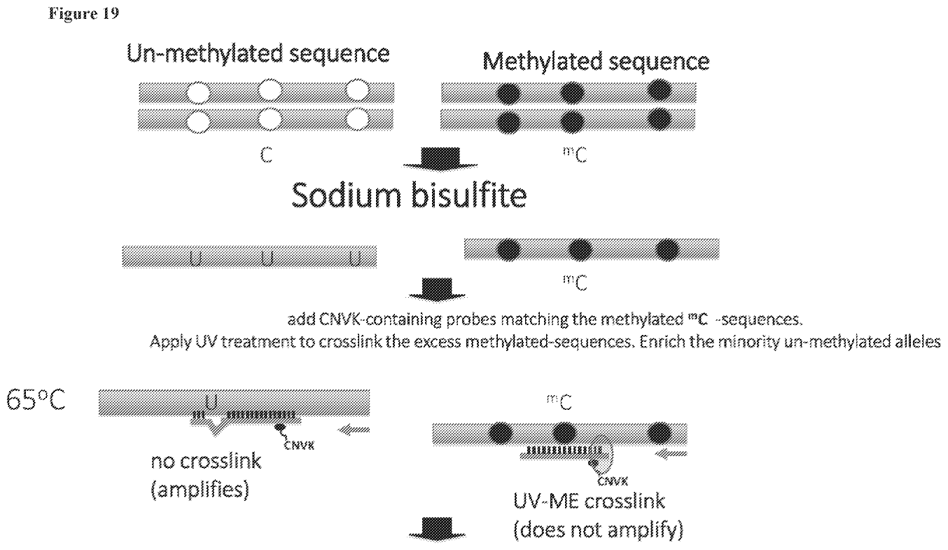

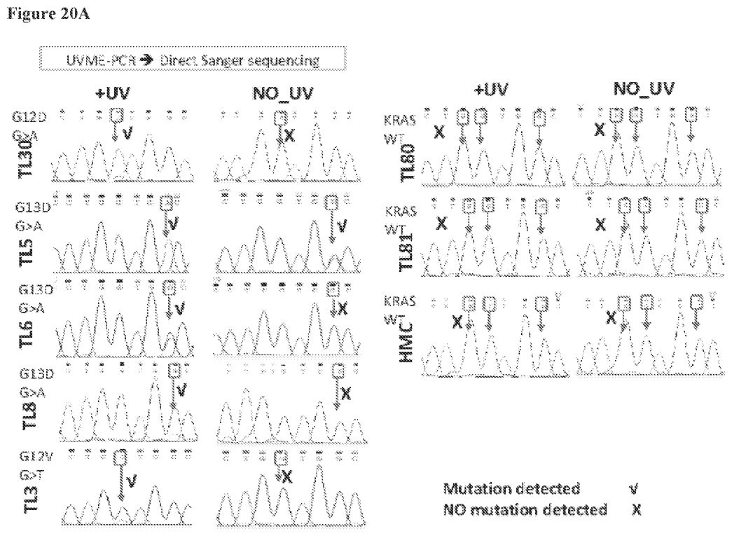

In some embodiments, split-probes of generic design may be utilized in this approach (e.g., FIG. 18, which shows the mismatch-dependent approach applied to work with split-CNVK probes that bind to a commonly present tail added to the end of each target). The ‘tail’ is an oligonucleotide added to all DNA fragments either via ligation or via 1-2 cycles of PCR using primers containing this tail at the 5′end. The split-CNVK probes are designed to contain a target-dependent region, which matches the wild type sample at the position that is to be interrogated for mutations, a linker made of poly-adenines, and a CNVK generic region corresponding to the generic tail added to the end of each fragment. If there is a mutation, the probe will not be able to bind effectively to the target-dependent region because of the mismatch at the mutation position. However, the split probes will bind well to the wild type DNA fragments as the sequence matches the wild type sequence. In this case, the CNVK-containing region hybridize to the generic tail and will produce a cross-link upon UV irradiation. When a cross-link is produced, the cross-linked wild type fragment will not be amplified during subsequent PCR.

The strategy in FIG. 18 avoids having to synthesize a separate probe for each interrogated DNA target. As FIG. 18 shows, the generic probe may be modified and adapted to produce target-specific split-CNVK probes at will and without expensive steps. It should be appreciated that a split-probe according to this example may be generated with a photo-activatable molecule.

### Example 13: An Embodiment Comprising Application of UVME for Selective Enrichment of Methylated or Un-Methylated DNA Targets

Application for enrichment of methylated (or un-methylated) DNA alleles using the methods described herein may also be applied using the methods described herein. First, the DNA to be tested is subjected to a treatment that converts un-methylated C to T (e.g., by using bisulfite treatment; or methyl-seq treatment (New England Biolabs); or APOBEC).

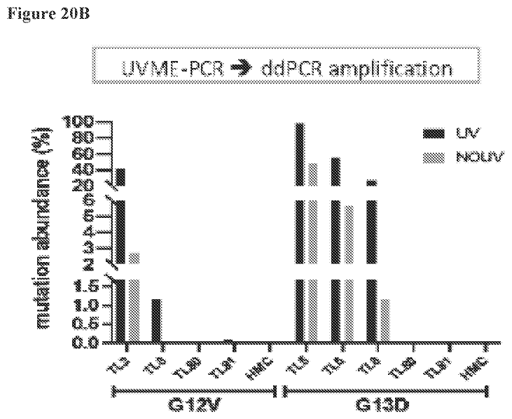

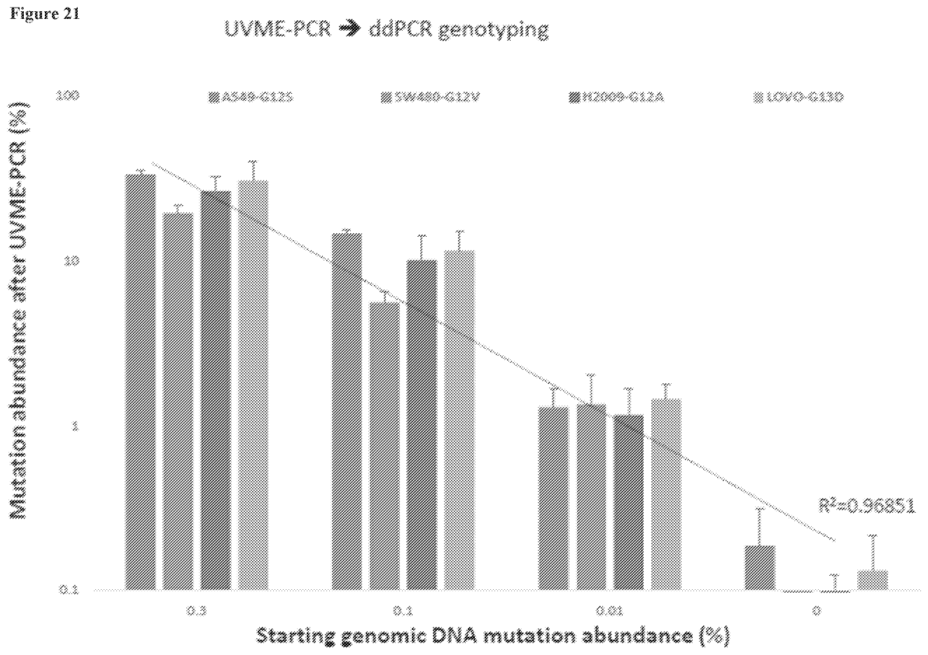

Once C is converted to T, the mismatch-dependent UVME approaches for enriching either the T-containing alleles or the C-containing alleles may be applied (FIG. 19). Therefore, the UVME method may also be applied towards enriching methylated or unmethylated DNA targets, following which they may be detected by sequencing, HRM, ddPCR or other methods.

An additional approach utilizing the methods disclosed herein also includes enrichment of either the methylated, or the unmethylated portion of a genomic fraction, by direct crosslinking of CNVK-containing probes to methylated Cs in the genome as opposed to non-methylated C that do not crosslink or crosslink to a much lesser extent relative to methylated Cs. This approach provides some advantages compared to the bisulfite-treatment approach as previously discussed in that DNA remains relatively intact and does not degrade as a result of chemical treatment prior to methylation detection. The direct approach described here avoids this type of sample degradation.

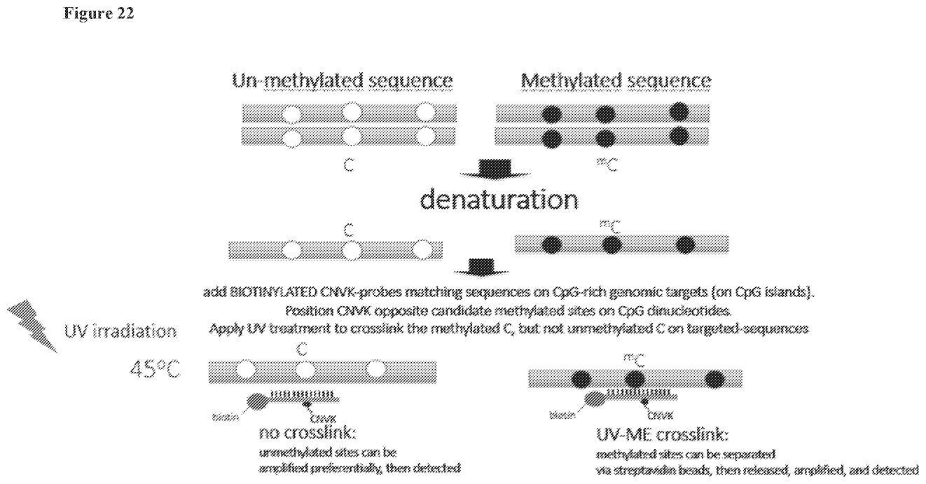

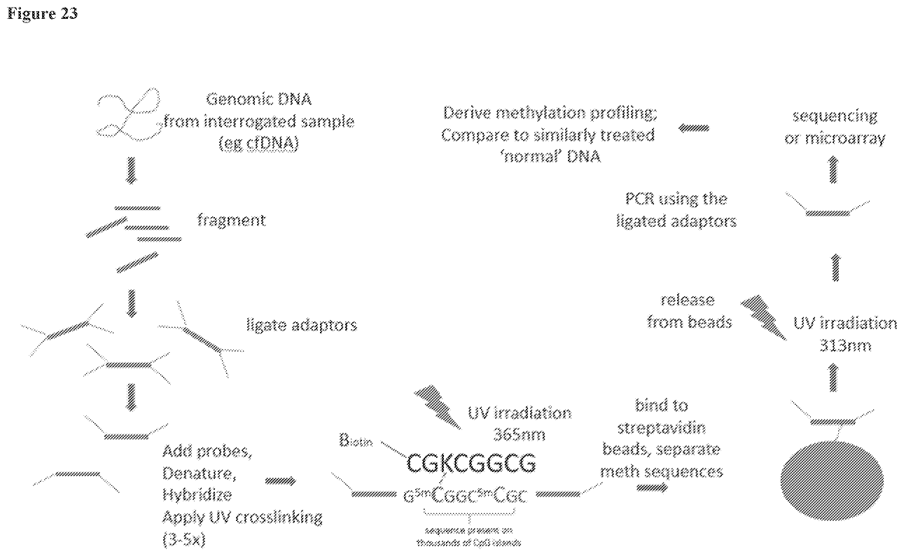

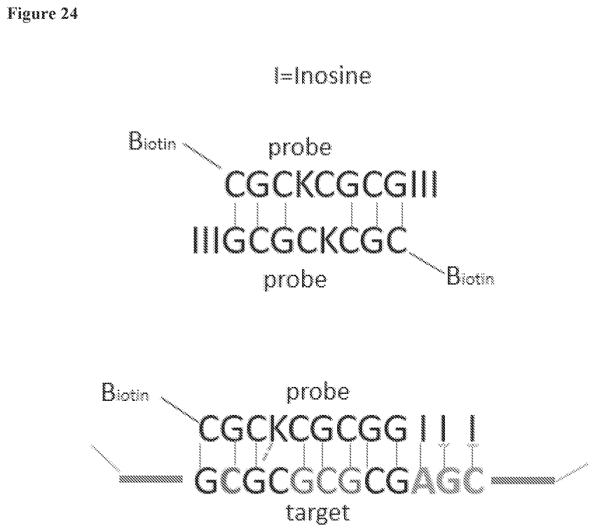

A representative, non-limiting example of this approach, which takes advantage of the differential reactivity of CNVK towards methylated C, relative to unmethylated C, is shown on FIG. 22. One or more biotinylated oligonucleotides containing CNVK opposite putative methylated Cs, for example within CpG dinucleotides, are synthesized. Following denaturation of the DNA sample, the probes anneal to their target(s) in the genome. Application of UV irradiation results in crosslinking of methylated Cs, while leaving unmethylated Cs intact. Accordingly, the methylated Cs crosslink to, for example, a biotinylated short probe which can then be separated and isolated, for example by binding to streptavidin beads and separated from unmethylated targets. One may then amplify either the methylated portion of the genome thus obtained, or alternatively, one may amplify the unmethylated portion of the genome following crosslinking of the methylated portion of the genome. Amplification may be accomplished by any method known in the art, for example by direct PCR. FIG. 23 shows an example workflow that allows the methylated portion of the genome to be preferentially detected via sequencing or microarrays.

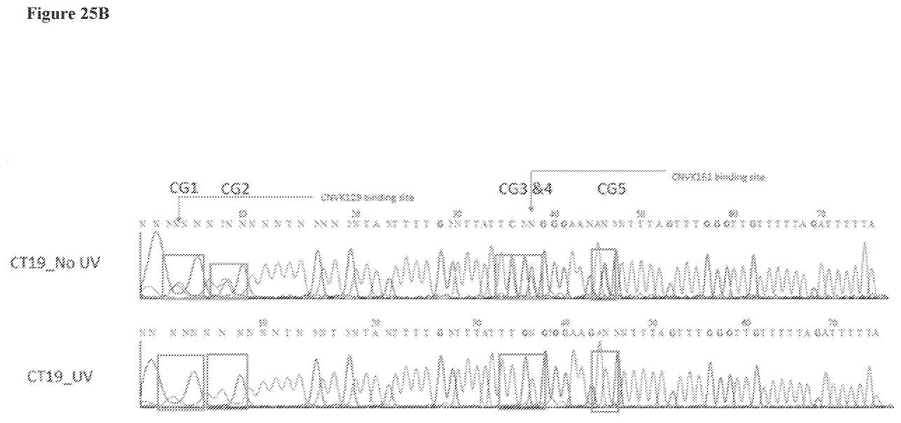

FIG. 25 shows a non-limiting, representative embodiment of selective enrichment of an unmethylated target gene (e.g., MAGE) among an excess of methylated target genes following selective crosslinking of the methylated alleles in a model system. As can be seen on FIG. 25, Sanger sequencing performed after selective crosslinking of an initial 1% ratio (unmethylated/meth alleles) results in a 60-100% ratio following enrichment of the un-methylated allele. To achieve this enrichment two different CNVK oligonucleotides directed to methylated CG positions at different parts of the same target sequence were used. Since methylation usually takes place on contiguous CG positions in cells, the ordinarily skilled artisan understands that CG positions within 10-1000 bp will be simultaneously methylated. Therefore, two or more CNVK oligonucleotides along the same strand can be used to increase the discrimination between methylated and non-methylated sequences. In some embodiments, the methods disclosed herein further comprise an additional oligonucleotide (e.g., an oligonucleotide comprising a photo-activatable molecule), such as two or more, three or more, four or more or five or more oligonucleotides. The ordinarily skilled artisan readily understands that multiple (one or more) oligonucleotides disclosed herein may be used in the methods disclosed herein. Use of multiple probes may be used to improve the discrimination and or specificity of the methods disclosed herein. Therefore, in some embodiments, two or more, three or more, four or more, five or more, six or more, seven or more, eight or more, nine or more, or ten or more oligonucleotides disclosed herein may be used in the methods disclosed herein. In some embodiments, two or more, three or more, four or more, five or more, six or more, seven or more, eight or more, nine or more, or ten or more oligonucleotides disclosed herein may be used to target a single nucleic acid target sequence. In some embodiments, two or more, three or more, four or more, five or more, six or more, seven or more, eight or more, nine or more, or ten or more oligonucleotides disclosed herein may be used to target one or more nucleic acid target sequences.

In particular, FIG. 25 shows enrichment using two oligonucleotides. Unmethylated MAGE ultramers (sequences containing TGT) were synthesized by IDT and mixed with methylated MAGE ultramers (sequences containing AGA). The methylated alleles contained methylated CpG nucleotides. UVME probes (inset) containing CNVK were used to crosslink selectively the methylated sequences at the position of the CpG nucleotides via 8 cycles of the following cycle: UV irradiation 10 see then denaturation at 950C 1 sec. Following this, samples were PCR amplified in order to selectively amplify alleles that were not crosslinked by CNVK probes. Subsequent Sanger sequencing demonstrated strong enrichment of the unmethylated sequences (containing TGT) and thus validating the selective crosslinking of the methylated alleles (containing AGA). Enrichment was enabled by the use of two CNVK probes directed against different methylated C positions on a single target. Two CNVK probes were used to bind simultaneously different methylated C positions on MAGE A1. UV was then applied to selectively crosslink one or both methC, thus inhibiting amplification.

The following two oligonucleotides were used:

MAGE-A1_sense129:
5′-aagggcggcg^{CNV}Ktggaatattt-3′-pho
MAGE-A1_sense161:
5′-agacgtcttcccg^{CNV}Kgggtg-3′-pho

A similar protocol, without having to use the bead-binding step, can be followed to detect the unmethylated portion of the genome. For example, following UV-crosslinking of the methylated portion of the genome, PCR or any other amplification method which will preferentially amplify the unmethylated portion, which is not crosslinked, may be applied.

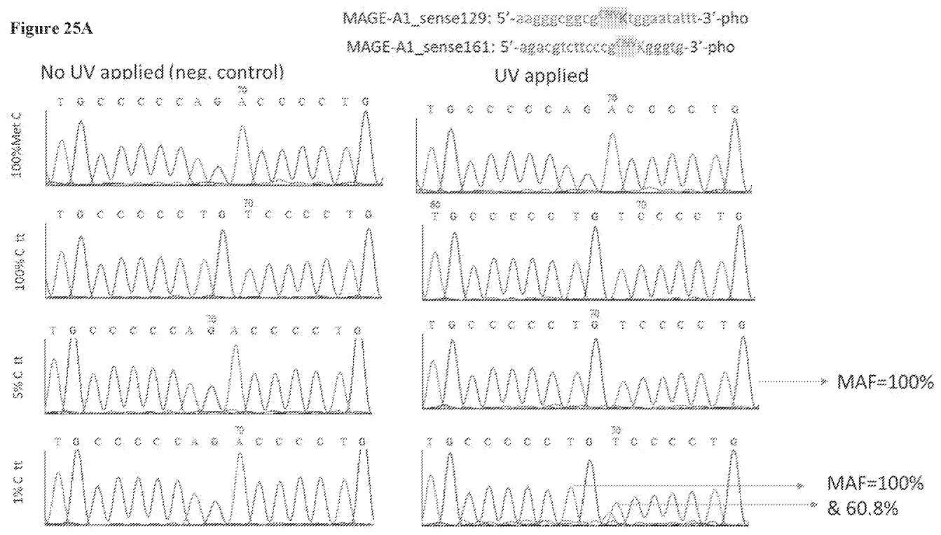

By synthesizing short probes that bind to a plurality of targets (e.g., CpG targets) in the genome, one may address the cytosine methylation status in thousands of genomic regions. For example, the probe sequence shown in FIG. 24 is known to exist in more than 8,000 CpG islands in the genome. Accordingly, following bead binding and sequencing one may interrogate thousands of CpG islands for their methylation status.

When designing short oligonucleotides of 15 nucleotides or less, in order to enable the probe to have a high melting temperature (Tm) and hybridize to the intended template at a given temperature, selected nucleotides can be substituted by modified nucleotides that raise the Tm of the oligonucleotide, such as peptide nucleic acid, locked nucleic acid, ‘super A,G,C,T’, or conjugation of intercalators that bind to the minor DNA groove and increase the Tm (e.g., minor groove binder (MGB probes)), or other synthetic base analogues that raise the Tm.

Because one may want to avoid excessive probe-to-probe binding in solution, while maximizing probe to target binding, a set of inosines can be added to the 3′ end of the CNVK-containing probes. Inosine pairs with all 4 DNA nucleotides, A, T, C and G. Therefore, the Tm (melting temperature) of probe-binding to target will be higher than the Tm of probe-probe binding in solution.

### Example 14: An Embodiment that Contemplates Extension of the Present Method to Different Photo-Activatable Molecules that have Base-Preference for Binding to DNA

There are several molecules that, when placed in the vicinity of DNA, upon photo-activation react with DNA. For example, psoralen and its derivatives have been known since the 80's to react with DNA upon intercalation with DNA or following incorporation into DNA upon covalent linkage of psoralen with a DNA base. Coumarin is another intercalator that may be photoactivated and reacts with DNA.

In the same way, CNVK was employed in the described UVME approach to provide base-specific cross-linking via UV irradiation, for enriching mutations in DNA, one can employ different photo-activatable molecules in the same approach. These include, without limitation, all known psoralen derivatives, some of which are known to show base-specific preference in UV-based cross-linking. Coumarin derivatives that show high preference towards cross-linking with thymidine in the opposite DNA strand may be used. Photosensitive dyes like acridine orange; or molecules like acetophenone/benzophenone may also cross-link to DNA. Accordingly, it is anticipated that the present UVME method is of general applicability and based on base-specificity for UV cross-linking may be adapted to enrichment of hotspot mutation-containing DNA in the human genome.

More details regarding exemplary photo-activatable molecules may be found at least in Boyer, V., Moustacchi, E. and Sage, E. (1988) Sequence specificity in photoreaction of various psoralen derivatives with DNA: role in biological activity. Biochemistry, 27, 3011-3018; Gia, O., Magno, S. M., Garbesi, A., Colonna, F. P. and Palumbo, M. (1992) Sequence specificity of psoralen photobinding to DNA: a quantitative approach. Biochemistry, 31, 11818-11822; Haque, M. M., Sun, H., Liu, S., Wang, Y. and Peng, X. (2014) Photoswitchable formation of a DNA interstrand cross-link by a coumarin-modified nucleotide. Angew Chem Int Ed Engl, 53, 7001-7005; and Jakubovska, J., Tauraitė, D. and Meškys, R. (2018) A versatile method for the UVA-induced cross-linking of acetophenone-or benzophenone-functionalized DNA. Sci Rep, 8, 16484.

### Example 15: Pre-PCR UV-Mediated Cross-Linking Minor-Allele Enrichment (Pre-PCR UVME)

Pre-PCR UV-mediated cross-linking minor-allele enrichment is contemplated. In one non-limiting, exemplary method, intact genomic DNA is fragmented with, for example, dsDNA Shearase Plus (Zymo Research) and, optionally, quantified. Mutation enrichment and UV irradiation protocol is then applied. After UV irradiation, half (5 μl) of the UV-treated sample or NO-UV control were added into a PCR reaction for a final volume of 25 μl containing 0.8X LC green (BioFire Diagnostics), 1×AmpliTaqGold™ buffer, 0.8 mM dNTP, 0.2 mM forward and reverse primers, 2 mM MgCl₂, 2 μl GC enhancer and 0.125 μl AmpiTaq™ polymerase (Thermo Fisher Scientific). PCR amplification was performed with an initial denaturation at 95° C. for 2 min, followed by 45 cycles of 30 see denaturation at 95° C., 30 see annealing at 57° C. and 10s elongation at 72° C.

### INCORPORATION BY REFERENCE

The contents of all references, patent applications, patents, and published patent applications, as well as the Figures and the Sequence Listing, cited throughout this application are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method comprising:
a) placing a photo-reactive molecule in an oligonucleotide in a position suitable for reaction with a non-target nucleic acid sequence present within a population of nucleic acid molecules upon hybridization of the oligonucleotide to the non-target nucleic acid sequence, wherein
1) the photo-reactive molecule has reaction-selectivity towards specific nucleotides, optionally wherein the nucleotides are pyrimidines; and
2) the placement of the photoreactive molecule is opposite position −1, position +1, and/or position zero of a putative alteration following hybridization of the oligonucleotide to a nucleic acid sequence, thereby generating a sequence mismatch at the DNA base-pair right next to the photoactivatable molecule due to the putative alteration;
b) applying light to the population of hybridized oligonucleotide-nucleic acid molecules at a wavelength adapted to induce a covalent cross-link between the photo-activatable molecule and nucleic acid molecules having non-target allele sequence when said nucleic acid molecules having non-target allele sequences are present in the population of nucleic acid molecules; and
c) amplifying the population of nucleic acid molecules to form a detectable number of amplified nucleic acid sequences, wherein the presence of the covalent cross-link between the photo-activatable molecule and nucleic acid molecules having non-target allele sequences when said nucleic acid molecules having non-target allele sequences are present in the population of nucleic acid molecules inhibits amplification thereof, optionally, wherein the amplification is digital amplification and/or detecting the amplified nucleic acid molecules;
wherein the method further comprises a step of nucleic acid amplification before oligonucleotide hybridization.

2. A method of selectively enriching nucleic acid molecules having a target allele sequence within a population of nucleic acid molecules, comprising:
a) hybridizing the nucleic acid molecules with an oligonucleotide that
1) comprises a sequence substantially complementary to at least a portion common between the target allele sequence and a non-target allele sequence; and
2) comprises a photo-activatable molecule, optionally wherein the photo-activatable molecule is selected from the group consisting of
i) a photo-activatable nucleoside that placed at a sequence position that is opposite a pyrimidine at nucleotide position −1 on the strand of a nucleic acid molecule having a non-target allele sequence hybridized to the oligonucleotide, thereby generating a sequence mismatch at the DNA base-pair right next to the photoactivatable molecule due to the putative alteration;
ii) a nucleoside conjugated to a coumarin-based or psoralen-based molecule that is complementary to a thymidine, cytosine, and/or a methylated cytosine at nucleotide position −1, +1, and/or zero on the strand of a nucleic acid molecule having a non-target allele sequence hybridized to the oligonucleotide, thereby generating a sequence mismatch at the DNA base-pair right next to the photoactivatable molecule due to the putative alteration; and
iii) a nucleoside conjugated to acetophenone, benzophenone, and/or acridine orange molecule that is complementary to a thymidine thymidine, cytosine, and/or a methylated cytosine at nucleotide position −1, +1, and/or zero on the strand of a nucleic acid molecule having a non-target allele sequence hybridized to the oligonucleotide, thereby generating a sequence mismatch at the DNA base-pair right next to the photoactivatable molecule due to the putative alteration;
b) applying light to the population of hybridized oligonucleotide-nucleic acid molecules at a wavelength adapted to induce a covalent cross-link between the photo-activatable molecule and nucleic acid molecules having non-target allele sequence when said nucleic acid molecules having non-target allele sequences are present in the population of nucleic acid molecules; and
c) amplifying the population of nucleic acid molecules to form a detectable number of amplified nucleic acid sequences, wherein the presence of the covalent cross-link between the photo-activatable molecule and nucleic acid molecules having non-target allele sequences when said nucleic acid molecules having non-target allele sequences are present in the population of nucleic acid molecules inhibits amplification thereof, optionally, wherein the amplification is digital amplification,
thereby selectively enriching the nucleic acid molecules having a target allele sequence within the population of nucleic acid molecules;
wherein the method further comprises a step of nucleic acid amplification before oligonucleotide hybridization.

3. The method of claim 1, wherein the nucleic acid amplification comprises polymerase chain reaction (PCR), optionally wherein nucleic acid molecules having target sequences are exponentially amplified and nucleic acid molecules having non-target allele sequences are linearly amplified when said nucleic acid molecules having non-target allele sequences are present in the population of nucleic acid molecules.

4. The method of claim 3, wherein the PCR amplification is selected from the group consisting of COLD-PCR, touch-down PCR, arbitrarily-primed PCR (AP-PCR), quantitative reverse transcription PCR (RT-qPCR), digital PCR (dPCR), asymmetric PCR, and solid-support based PCR.

5. The method of claim 1, wherein the nucleic acid amplification comprises isothermal amplification selected from the group consisting of recombinase-polymerase amplification (RPA), LAMP-isothermal amplification, or strand displacement amplification.

6-7. (canceled)

8. The method of claim 1, wherein the nucleic acid amplification comprises polymerase chain reaction (PCR) to form a detectable number of amplified nucleic acid sequences.

9. The method of claim 8, wherein the step of nucleic acid amplification prior to oligonucleotide hybridization comprises amplifying the population of nucleic acid molecules with polymerase chain reaction (PCR) using a single amplification primer comprising a 5′ tail and DNA polymerase to form a detectable number of nucleic acid sequences comprising the target allele sequence.

10. The method of claim 9, wherein the step of nucleic acid amplification before oligonucleotide hybridization comprises using terminal deoxynucleotidyl-transferase to add poly-adenine tails to the 3′ end of the nucleic acid molecules.

11-19. (canceled)

20. The method of claim 1, wherein the method further comprises treating the nucleic acid molecules prior to oligonucleotide hybridization with bisulfite, methyl-seq, or APOBEC.

21-23. (canceled)

24. The method of claim 1, further comprising adding a nucleotide tail to the nucleic acid molecules prior to oligonucleotide hybridization, optionally wherein the nucleotide tail addition is performed through ligation or by extension with a PCR primer.

25. (canceled)

26. A method of selectively enriching nucleic acid molecules having a target allele sequence within a population of nucleic acid molecules, comprising:
a) hybridizing the nucleic acid molecules with an oligonucleotide that comprises:
1) a sequence substantially complementary to at least a portion common between the target allele sequence and a non-target allele sequence; and
2) a photo-activatable molecule, optionally wherein the photo-activatable molecule is selected from the group consisting of
i) a photo-activatable nucleoside that placed at a sequence position that is opposite a pyrimidine at nucleotide position −1 on the strand of a nucleic acid molecule having a non-target allele sequence hybridized to the oligonucleotide, thereby generating a sequence mismatch at the DNA base-pair right next to the photoactivatable molecule due to the putative alteration;
ii) a nucleoside conjugated to a coumarin-based or psoralen-based molecule that is complementary to a thymidine, cytosine, and/or a methylated cytosine at nucleotide position −1, +1, and/or zero on the strand of a nucleic acid molecule having a non-target allele sequence hybridized to the oligonucleotide, thereby generating a sequence mismatch at the DNA base-pair right next to the photoactivatable molecule due to the putative alteration; and
iii) a nucleoside conjugated to acetophenone, benzophenone, and/or acridine orange molecule that is complementary to a thymidine, cytosine, and/or a methylated cytosine at nucleotide position −1, +1, and/or zero on the strand of a nucleic acid molecule having a non-target allele sequence hybridized to the oligonucleotide, thereby generating a sequence mismatch at the DNA base-pair right next to the photoactivatable molecule due to the putative alteration, and
b) if the oligonucleotide comprising the photo-activatable molecule hybridizes to the strand of the nucleic acid molecule having the non-target allele sequence,
i) applying light to the population of hybridized oligonucleotide-nucleic acid molecules at a wavelength adapted to induce a covalent cross-link between the photo-activatable molecule and nucleic acid molecules having non-target allele sequence when said nucleic acid molecules having non-target allele sequences are present in the population of nucleic acid molecules; and
ii) removing the non-target sequences cross-linked to the oligonucleotide, thereby enriching the nucleic acid molecules having a target allele sequence within the population of nucleic acid molecules, or
c) if the oligonucleotide comprising the photo-activatable molecule hybridizes to the strand of the nucleic acid molecule having the target allele sequence,
i) applying light to the population of hybridized oligonucleotide-nucleic acid molecules at a wavelength adapted to induce a covalent cross-link between the photo-activatable molecule and nucleic acid molecules having target allele sequence;
ii) removing the target sequences cross-linked to the oligonucleotide; and
iii) applying light to the population of hybridized oligonucleotide-nucleic acid molecules at a wavelength adapted to reverse the covalent cross-link between the photo-activatable molecule and nucleic acid molecules having target allele sequence, thereby enriching the nucleic acid molecules having a target allele sequence within the population of nucleic acid molecules;
wherein the method further comprises
d) a step of nucleic acid amplification before oligonucleotide hybridization, wherein the nucleic acid amplification comprises polymerase chain reaction (PCR) using a single amplification primer comprising a 5′ tail and DNA polymerase to form a detectable number of nucleic acid sequences comprising the target allele sequence; and/or
e) a step of amplifying the nucleic acid molecules having the target allele sequence after steps (b)(ii) or (c)(iii).

27-29. (canceled)

30. The method of claim 26, wherein the step of nucleic acid amplification before oligonucleotide hybridization comprises using terminal deoxynucleotidyl-transferase to add poly-adenine tails to the 3′ end of the nucleic acid molecules.

31-33. (canceled)

34. The method of claim 26, wherein the allele is selected from the group consisting of a single nucleotide polymorphism (SNP), a micro-deletion, and an insertion.

35. The method of claim 26, wherein the target allele sequence and the non-target allele sequence differ by more than one single nucleotide.

36. The method of claim 26, wherein the method further comprises a incorporating a modified DNA base into the nucleic acid molecules prior to oligonucleotide hybridization, optionally wherein the modified DNA base is selected from the group consisting of methylated deoxy-cytosine-triphosphate (d5mCTP), deoxyuridine triphosphate (dUTP), deoxyinosine triphosphate (dITP), 6N-methyladenine triphosphate, 8-oxo-guanine triphosphate, 4N-methylcytosine triphosphate, 5-substituted pyrimidine trisphosphate and 7-substituted 7-deazapurine triphosphate.

37. The method of claim 26, wherein the method further comprises treating the nucleic acid molecules prior to oligonucleotide hybridization with bisulfite, methyl-seq, or APOBEC.

38. The method of claim 26, the method further comprising a step of generating the single-stranded nucleic acid molecules by denaturing double-stranded nucleic acid molecules, optionally wherein the double-stranded nucleic acid molecules are genomic DNA.

39. The method of claim 26, wherein the oligonucleotide is immobilized on a solid support, optionally wherein the solid support is a bead.

40. The method of claim 26, wherein the oligonucleotide is biotinylated.

41-48. (canceled)

49. The method of claim 1, wherein the methods comprise enriching for multiple target sequences, at least one target nucleic acid sequence is a portion of an oncogene selected from KRAS, BRAF and TP53.

50-65. (canceled)
